SEQUENCE LISTING (2 sequences)

SEQ ID NO: 1 - RNA, 20 nt, Escherichia coli
  source 1..20: mol_type = other RNA; note = K-12 MG1655
ggctgcgtatagccaaagcg

SEQ ID NO: 2 - RNA, 20 nt, Escherichia coli
  source 1..20: mol_type = other RNA; note = K-12 MG1655
ggtatgttaatcactctgcg

(19) United States
(12) Patent Application Publication
(10) Pub. No.: US 2026/0242837 A1
(43) Pub. Date: Aug. 20, 2026

(54) PRODUCTION OF A SACCHARIDE BY A CELL WITH REDUCED SYNTHESIS OF LACTOBIONIC ACID

(71) Applicant: INBIOSE N.V., Zwijnaarde (BE)

(72) Inventors: Joeri Beauprez, Zwijnaarde (BE); Thomas Decoene, Zwijnaarde (BE); Annelies Vercauteren, Zwijnaarde (BE)

(21) Appl. No.: 19/153,584

(22) PCT Filed: Feb. 6, 2024
(86) PCT No.: PCT/EP2024/052838

§ 371 Date: Aug. 4, 2025

(30) Foreign Application Priority Data
Feb. 6, 2023 (EP) 23155144.1
Feb. 6, 2023 (LU) LU503441

(51) Int. Cl. C12P 19/12; C12N 9/04; C12N 15/52; C12P 19/04

(52) U.S. Cl. CPC C12P 19/12; C12N 9/0006; C12N 15/52; C12P 19/04; C12Y 101/05002

(57) ABSTRACT

A cell for production of a saccharide wherein synthesis of lactobionic acid in the cell is rendered less functional or is knocked out. The disclosure further provides use of the cell in a cultivation or incubation. The disclosure also describes methods for the production of a saccharide using the cell as well as the purification of the saccharide.

### CROSS-REFERENCE TO RELATED APPLICATIONS

This application is a national phase entry under 35 U.S.C. § 371 of International Patent Application PCT/EP2024/052838, filed Feb. 6, 2024, designating the United States of America and published as International Patent Publication WO 2024/165524 A1 on Aug. 15, 2024, which claims the benefit under Article 8 of the Patent Cooperation Treaty of European Patent Application Serial No. 23155144.1, filed Feb. 6, 2023, and to Luxembourg Patent Application No. LU503441, also filed on Feb. 6, 2023.

### STATEMENT REGARDING ELECTRONIC FILING OF A SEQUENCE LISTING

The XML file named “072-PCT Sequence Listing.xml” created on Feb. 5, 2024, with a size of 3,141 bytes is hereby incorporated herein in its entirety by this reference.

### TECHNICAL FIELD

The disclosure is in the technical field of synthetic biology, metabolic engineering and cell cultivation. The disclosure provides a cell for production of a saccharide wherein synthesis of lactobionic acid in the cell is rendered less functional or is knocked out. The disclosure further provides use of the cell in a cultivation or incubation. The disclosure also describes methods for the production of a saccharide using the cell as well as the purification of the saccharide.

### BACKGROUND

Saccharides like, e.g., disaccharides and oligosaccharides, are very diverse in chemical structure and are composed of miscellaneous monosaccharides, such as, e.g., glucose, galactose, N-acetylglucosamine, xylose, rhamnose, fucose, mannose, N-acetylneuraminic acid, N-acetylgalactosamine, galactosamine, glucosamine, glucuronic acid, galacturonic acid. Saccharides are widely distributed in all living organisms and play important roles in a variety of physiological and pathological processes, such as cell metastasis, signal transduction, intercellular adhesion, inflammation, and immune response. Economical production of these saccharides is of utmost importance to fully benefit of their biological advantages. An important group of saccharides comprises mammalian milk oligosaccharides (MMOs) and human milk oligosaccharides (HMOs) found in mammalian and human milk, respectively. A wide variety of synthesis methods have been developed already, ranging from extraction over chemical synthesis to enzymatic synthesis. These methods are currently least applied, whereas biotechnological fermentative production is nowadays pursued and commercialized. Methods for the production of saccharides, in particular oligosaccharides, are well known for a person skilled in the art like, e.g., described in Faijes et al. (2019), US2010120096A, JP2013201913, and WO2022/034067).

Lactobionic acid (LBA) can be produced by oxidation of lactose due to a dehydrogenase (lactose-oxidase) system into lactone that is further hydrolyzed into LBA. LBA can also be produced upon oxidation of maltose. LBA can also be produced by lactose dehydrogenase and lactonase. LBA can also be formed out of other saccharides than lactose like, e.g., but not limited to D-glucose, D-galactose, D-mannose, D-talose, D-xylose, D-ribose, L-arabinose, cellobiose and D-fructose. LBA is classified as a bionic acid, chemically constituted of a gluconic acid bonded to a galactose. LBA is known for its antioxidant, antimicrobial, chelating, stabilizer, acidulant, and moisturizing properties. Although industry has high interest in LBA, applications with LBA are rare. Ingestion of LBA is still considered not safe due to the absence of testing and knowledge on health impact, though in vivo tests are being initialized.

### BRIEF SUMMARY

Provided are tools and methods by means of which a saccharide can be produced, preferably in an efficient, time and cost-effective way and that yields high amounts of the desired saccharide with small to zero amounts of LBA, a modified form of LBA, a glycosylated form of LBA, fucosylated LBA and/or sialylated LBA.

Provided are methods and a cell for the production of a saccharide. The disclosure also provides methods for the purification of the saccharide. Furthermore, the disclosure provides a cell that is genetically engineered as described herein and wherein synthesis of LBA, a modified form of LBA, a glycosylated form of LBA, fucosylated LBA and/or sialylated LBA is rendered less functional or is knocked out. This disclosure also provides a purified saccharide by the above-referenced process. Further benefits of the teachings of this disclosure will be apparent to one skilled in the art from reading this disclosure.

### Definitions

The words used in this specification to describe the disclosure and its various embodiments are to be understood not only in the sense of their commonly defined meanings, but to include by special definition in this specification structure, material or acts beyond the scope of the commonly defined meanings. Thus, if an element can be understood in the context of this specification as including more than one meaning, then its use in a claim must be understood as being generic to all possible meanings supported by the specification and by the word itself.

The various aspects and embodiments of the disclosure described herein are to be understood not only in the order and context specifically described in this specification, but to include any order and any combination thereof. Each embodiment as identified herein may be combined together unless otherwise indicated. All publications, patents, and patent applications mentioned in this specification are herein incorporated by reference to the same extent as if each individual publication, patent, or patent application was specifically and individually indicated to be incorporated by reference in its entirety. Unless specifically stated otherwise, all words used in the singular number shall be deemed to include the plural and vice versa. Unless defined otherwise, all technical and scientific terms used herein generally have the same meaning as commonly understood by one of ordinary skill in the art to which this disclosure belongs. Generally, the nomenclature used herein and the laboratory procedures in cell culture, molecular genetics, organic chemistry and nucleic acid chemistry and hybridization described herein are those well-known and commonly employed in the art. Standard techniques are used for nucleic acid and peptide synthesis. Generally, enzymatic reactions and purification steps are performed according to the manufacturer's specifications.

In the specification, there have been disclosed embodiments of the disclosure, and although specific terms are employed, the terms are used in a descriptive sense only and not for purposes of limitation, the scope of the disclosure being set forth in the following claims. It must be understood that the illustrated embodiments have been set forth only for the purposes of example and that it should not be taken as limiting the disclosure. It will be apparent to those skilled in the art that alterations, other embodiments, improvements, details and uses can be made consistent with the letter and spirit of the disclosure herein and within the scope of this disclosure, which is limited only by the claims, construed in accordance with the patent law, including the doctrine of equivalents. In the claims that follow, reference characters used to designate claim steps are provided for convenience of description only and are not intended to imply any particular order for performing the steps, unless specifically stated otherwise.

Throughout the application, unless explicitly stated otherwise, the features “synthesize,” “synthesized” and “synthesis” are interchangeably used with the features “produce,” “produced” and “production,” respectively. Throughout the application, unless explicitly stated otherwise, the expressions “capable of . . . <verb>” and “capable to . . . <verb>” are preferably replaced with the active voice of the verb and vice versa. For example, the expression “capable of expressing” is preferably replaced with “expresses” and vice versa, i.e., “expresses” is preferably replaced with “capable of expressing.” In this document and in its claims, the verb “to comprise,” “to have” and “to contain” and their conjugations are used in their non-limiting sense to mean that items following the word are included, but items not specifically mentioned are not excluded. Throughout the application, the verb “to comprise” may be replaced by “to consist” or “to consist essentially of” and vice versa. In addition, the verb “to consist” may be replaced by “to consist essentially of” meaning that a composition as defined herein may comprise additional component(s) than the ones specifically identified, the additional component(s) not altering the unique characteristic of the disclosure. In this document and in its claims, unless specifically stated otherwise, the verbs “to comprise,” “to have” and “to contain,” and their conjugations, may be replaced by “to consist of” (and its conjugations) or “to consist essentially of” (and its conjugations) and vice versa. In addition, reference to an element by the indefinite article “a” or “an” does not exclude the possibility that more than one of the elements is present, unless the context clearly requires that there be one and only one of the elements. The indefinite article “a” or “an” thus usually means “at least one.”

Throughout the document and in the claims, unless explicitly stated otherwise, the articles “a” and “an” are preferably replaced by “at least one,” more preferably “at least two,” even more preferably by “at least three,” even more preferably by “at least four,” even more preferably by “at least five,” even more preferably by “at least six,” most preferably by “at least two.” The word “about” or “approximately” when used in association with a numerical value (e.g., “about 10”) or with a range (e.g., “about x to approximately y”) preferably means that the value or range is interpreted as being as accurate as the method used to measure it. If no error margins are specified, the expression “about” or “approximately” when used in association with a numerical value is interpreted as having the same round-off as the given value. Throughout this document and its claims, unless otherwise stated, the expression “from x to y,” wherein x and y represent numerical values, refers to a range of numerical values wherein x is the lower value of the range and y is the upper value of the range. Herein, x and y are also included in the range.

According to the disclosure, the term “polynucleotide(s)” generally refers to any polyribonucleotide or polydeoxyribonucleotide, which may be unmodified RNA or DNA or modified RNA or DNA. DNAs or RNAs comprising unusual bases, such as inosine, or modified bases, such as tritylated bases, are to be understood to be covered by the term “polynucleotides.” It will be appreciated that a great variety of modifications have been made to DNA and RNA that serve many useful purposes known to those of skill in the art. The term “polynucleotide(s)” as it is employed herein embraces such chemically, enzymatically or metabolically modified forms of polynucleotides, as well as the chemical forms of DNA and RNA characteristic of viruses and cells, including, for example, simple and complex cells. The term “polynucleotide(s)” also embraces short polynucleotides often referred to as oligonucleotide(s).

“Polypeptide(s)” refers to any peptide or protein comprising two or more amino acids joined to each other by peptide bonds or modified peptide bonds. “Polypeptide(s)” refers to both short chains, commonly referred to as peptides, oligopeptides and oligomers and to longer chains generally referred to as proteins. Polypeptides may contain amino acids other than the 20 gene encoded amino acids. “Polypeptide(s)” include those modified either by natural processes, such as processing and other post-translational modifications, but also by chemical modification techniques as well known to the skilled person. The same type of modification may be present in the same or varying degree at several sites in a given polypeptide. Furthermore, a given polypeptide may contain many types of modifications. Modifications can occur anywhere in a polypeptide, including the peptide backbone, the amino acid sidechains, and the amino or carboxyl termini. Polypeptides may be branched or cyclic, with or without branching. Cyclic, branched and branched circular polypeptides may result from post-translational natural processes and may be made by entirely synthetic methods, as well.

The term “polynucleotide encoding a polypeptide” as used herein encompasses polynucleotides that include a sequence encoding a polypeptide of the disclosure. The term also encompasses polynucleotides that include a single continuous region or discontinuous regions encoding the polypeptide (for example, interrupted by integrated phage or an insertion sequence or editing) together with additional regions that also may contain coding and/or non-coding sequences.

“Isolated” means altered “by the hand of man” from its natural state, i.e., if it occurs in nature, it has been changed or removed from its original environment, or both. For example, a polynucleotide or a polypeptide naturally present in a living organism is not “isolated,” but the same polynucleotide or polypeptide separated from the coexisting materials of its natural state is “isolated,” as the term is employed herein. Similarly, a “synthetic” sequence, as the term is used herein, means any sequence that has been generated synthetically and not directly isolated from a natural source. “Synthesized,” as the term is used herein, means any synthetically generated sequence and not directly isolated from a natural source.

“Recombinant” means genetically engineered DNA prepared by transplanting or splicing genes from one species into the cells of a host organism of a different species. Such DNA becomes part of the host's genetic makeup and is replicated.

The terms “recombinant” or “transgenic” or “metabolically engineered” or “genetically engineered,” as used herein with reference to a cell or host cell, are used interchangeably and indicates that the cell replicates a heterologous nucleic acid, or expresses a peptide or protein encoded by a heterologous nucleic acid (i.e., a sequence “foreign to the cell” or a sequence “foreign to the location or environment in the cell”). Such cells are described to be transformed with at least one heterologous or exogenous gene or are described to be transformed by the introduction of at least one heterologous or exogenous gene. Recombinant or metabolically engineered or genetically engineered or transgenic cells can contain genes that are not found within the native (non-recombinant) form of the cell. Recombinant cells can also contain genes found in the native form of the cell wherein the genes are modified and re-introduced into the cell by artificial means. The terms also encompass cells that contain a nucleic acid endogenous to the cell that has been modified or its expression or activity has been modified without removing the nucleic acid from the cell; such modifications include those obtained by gene replacement, replacement of a promoter; site-specific mutation; CrispR; riboswitch; recombineering; ssDNA mutagenesis; transposon mutagenesis and related techniques as known to a person skilled in the art. Accordingly, a “recombinant polypeptide” is one that has been produced by a recombinant cell. The terms also encompass cells that have been modified by removing a nucleic acid endogenous to the cell by means of common well-known technologies for a skilled person (like, e.g., knocking-out genes).

A “heterologous sequence” or a “heterologous nucleic acid,” as used herein, is one that originates from a source foreign to the particular cell (e.g., from a different species), or, if from the same source, is modified from its original form or place in the genome. Thus, a heterologous nucleic acid operably linked to a promoter is from a source different from that from which the promoter was derived, or, if from the same source, is modified from its original form or place in the genome. The heterologous sequence may be stably introduced, e.g., by transfection, transformation, conjugation or transduction, into the genome of the host cell, wherein techniques may be applied that will depend on the cell and the sequence that is to be introduced. Various techniques are known to a person skilled in the art and are, e.g., disclosed in Sambrook et al., Molecular Cloning: A Laboratory Manual, 2nd Ed., Cold Spring Harbor Laboratory Press, Cold Spring Harbor, N.Y. (1989). The term “mutant” or “engineered” cell as used within the context of the disclosure refers to a cell that is genetically engineered.

The term “endogenous” within the context of the present disclosure refers to any polynucleotide, polypeptide or protein sequence that is a natural part of a cell and is occurring at its natural location in the cell chromosome and of which the control of expression has not been altered compared to the natural control mechanism acting on its expression. The term “exogenous” refers to any polynucleotide, polypeptide or protein sequence that originates from outside the cell under study and not a natural part of the cell or that is not occurring at its natural location in the cell chromosome or plasmid.

The term “heterologous” when used in reference to a polynucleotide, gene, nucleic acid, polypeptide, or enzyme refers to a polynucleotide, gene, nucleic acid, polypeptide, or enzyme that is from a source or derived from a source other than the host organism. In contrast a “homologous” polynucleotide, gene, nucleic acid, polypeptide, or enzyme is used herein to denote a polynucleotide, gene, nucleic acid, polypeptide, or enzyme that is derived from the host organism. When referring to a gene regulatory sequence or to an auxiliary nucleic acid sequence used for maintaining or manipulating a gene sequence (e.g., a promoter, a 5′ untranslated region, 3′ untranslated region, poly A addition sequence, intron sequence, splice site, ribosome binding site, internal ribosome entry sequence, genome homology region, recombination site, etc.), “heterologous” means that the regulatory sequence or auxiliary sequence is not naturally associated with the gene with which the regulatory or auxiliary nucleic acid sequence is juxtaposed in a construct, genome, chromosome, or episome. Thus, a promoter operably linked to a gene to which it is not operably linked to in its natural state (i.e., in the genome of a non-genetically engineered organism) is referred to herein as a “heterologous promoter,” even though the promoter may be derived from the same species (or, in some cases, the same organism) as the gene to which it is linked.

The term “modified expression” of a gene relates to a change in expression compared to the wild-type expression of the gene in any phase of the production process of the desired saccharide. The modified expression is either a lower or higher expression compared to the wild-type, wherein the term “higher expression” is also defined as “overexpression” of the gene in the case of an endogenous gene or “expression” in the case of a heterologous gene that is not present in the wild-type strain. Lower expression is obtained by means of common well-known technologies for a skilled person (such as the usage of siRNA, CrispR, CrispRi, riboswitch, recombineering, homologous recombination, ssDNA mutagenesis, RNAi, miRNA, asRNA, mutating genes, knocking-out genes, transposon mutagenesis, etc.) that are used to change the genes in such a way that they are “less able” (i.e., statistically significantly “less able” compared to a functional wild-type gene) or completely unable (such as knocked-out genes) to produce functional final products. The term “riboswitch,” as used herein, is defined to be part of the messenger RNA that folds into intricate structures that block expression by interfering with translation. Binding of an effector molecule induces conformational change(s) permitting regulated expression post-transcriptionally. Next to changing the gene of interest in such a way that lower expression is obtained as described above, lower expression can also be obtained by changing the transcription unit, the promoter, an untranslated region, the ribosome binding site, the Shine Dalgarno sequence or the transcription terminator. Lower expression or reduced expression can, for instance, be obtained by mutating one or more base pairs in the promoter sequence or changing the promoter sequence fully to a constitutive promoter with a lower expression strength compared to the wild-type or an inducible promoter that results in regulated expression or a repressible promoter that results in regulated expression. Overexpression or expression is obtained by means of common well-known technologies for a skilled person (such as the usage of artificial transcription factors, de novo design of a promoter sequence, ribosome engineering, introduction or re-introduction of an expression module at euchromatin, usage of high-copy-number plasmids), wherein the gene is part of an “expression cassette” that relates to any sequence in which a promoter sequence, untranslated region sequence (containing either a ribosome binding sequence, Shine Dalgarno or Kozak sequence), a coding sequence and optionally a transcription terminator is present, and leading to the expression of a functional active protein. The expression is either constitutive or conditional or regulated or tuneable.

The term “constitutive expression” is defined as expression that is not regulated by transcription factors other than the subunits of RNA polymerase (e.g., the bacterial sigma factors like s⁷⁰, s⁵⁴, or related s-factors and the yeast mitochondrial RNA polymerase specificity factor MTF1 that co-associate with the RNA polymerase core enzyme) under certain growth conditions. Non-limiting examples of such transcription factors are CRP, LacI, ArcA, Cra, IclR in E. coli, or, Aft2p, Crz1p, Skn7 in Saccharomyces cerevisiae, or, DeoR, GntR, Fur in B. subtilis. These transcription factors bind on a specific sequence and may block or enhance expression in certain growth conditions. The RNA polymerase is the catalytic machinery for the synthesis of RNA from a DNA template. RNA polymerase binds a specific DNA sequence to initiate transcription, for instance via a sigma factor in prokaryotic hosts or via MTF1 in yeasts. Constitutive expression offers a constant level of expression with no need for induction or repression.

The term “regulated expression” is defined as expression that is regulated by transcription factors other than the subunits of RNA polymerase (e.g., bacterial sigma factors) under certain growth conditions. Examples of such transcription factors are described above. Commonly expression regulation is obtained by means of an inducer, such as but not limited to IPTG, arabinose, rhamnose, fucose, allo-lactose or pH shifts, or temperature shifts or carbon depletion or substrates or the produced product.

The term “control sequences” refers to sequences recognized by the cells transcriptional and translational systems, allowing transcription and translation of a polynucleotide sequence to a polypeptide. Such DNA sequences are thus necessary for the expression of an operably linked coding sequence in a particular host cell, cell or organism. Such control sequences can be, but are not limited to, promoter sequences, ribosome binding sequences, Shine Dalgarno sequences, Kozak sequences, transcription terminator sequences. The control sequences that are suitable for prokaryotes, for example, include a promoter, optionally an operator sequence, and a ribosome binding site. Eukaryotic cells are known to utilize promoters, polyadenylation signals, and enhancers. DNA for a presequence or secretory leader may be operably linked to DNA for a polypeptide if it is expressed as a preprotein that participates in the secretion of the polypeptide; a promoter or enhancer is operably linked to a coding sequence if it affects the transcription of the sequence; or a ribosome binding site is operably linked to a coding sequence if it affects the transcription of the sequence; or a ribosome binding site is operably linked to a coding sequence if it is positioned so as to facilitate translation. The control sequences can furthermore be controlled with external chemicals, such as, but not limited to, IPTG, arabinose, lactose, allo-lactose, rhamnose or fucose via an inducible promoter or via a genetic circuit that either induces or represses the transcription or translation of the polynucleotide to a polypeptide.

Generally, “operably linked” means that the DNA sequences being linked are contiguous, and, in the case of a secretory leader, contiguous and in reading phase. However, enhancers do not have to be contiguous.

The term “wild-type” refers to the commonly known genetic or phenotypical situation as it occurs in nature.

The term “modified expression of a protein,” as used herein, refers to i) higher expression or overexpression of an endogenous protein, ii) expression of a heterologous protein, iii) expression and/or overexpression of a variant protein that has a higher activity compared to the wild-type (i.e., native in the expression host) protein, iv) reduced expression of an endogenous protein or v) expression and/or overexpression of a variant protein that has a reduced activity compared to the wild-type (i.e., native in the expression host) protein. Preferably, the term “modified expression of a protein,” as used herein, refers to i) higher expression or overexpression of an endogenous protein, ii) expression of a heterologous protein or iii) expression and/or overexpression of a variant protein that has a higher activity compared to the wild-type (i.e., native in the expression host) protein.

The term “modified activity” of a protein relates to a non-native activity of the protein in any phase of the production process of the desired saccharide. The term “non-native,” as used herein with reference to the activity of a protein, indicates that the protein has been modified to have an abolished, impaired, reduced, delayed, higher, accelerated or improved activity compared to the native activity of the protein. A modified activity of a protein is obtained by modified expression of the protein or is obtained by expression of a modified, i.e., mutant form of the protein. A mutant form of the protein can be obtained by expression of a mutant form of the gene encoding the protein, e.g., comprising a deletion, an insertion and/or a mutation of one or more nucleotides compared to the native gene sequence. A mutant form of a gene can be obtained by techniques well-known to a person skilled in the art, such as but not limited to site-specific mutation; CrispR; riboswitch; recombineering; ssDNA mutagenesis; transposon mutagenesis.

The term “non-native,” as used herein with reference to a cell producing a saccharide, indicates that the saccharide is i) not naturally produced or ii) when naturally produced not in the same amounts by the cell; and that the cell has been genetically engineered to be able to produce the saccharide or to have a higher production of the saccharide.

“Variant(s)” as the term is used herein, is a polynucleotide or polypeptide that differs from a reference polynucleotide or polypeptide respectively but retains essential properties. A typical variant of a polynucleotide differs in nucleotide sequence from another, reference polynucleotide. Changes in the nucleotide sequence of the variant may or may not alter the amino acid sequence of a polypeptide encoded by the reference polynucleotide. Nucleotide changes may result in amino acid substitutions, additions, deletions, fusions and truncations in the polypeptide encoded by the reference sequence, as discussed below. A typical variant of a polypeptide differs in amino acid sequence from another, reference polypeptide. Generally, differences are limited so that the sequences of the reference polypeptide and the variant are closely similar overall and, in many regions, identical. A variant and reference polypeptide may differ in amino acid sequence by one or more substitutions, additions, deletions in any combination. A substituted or inserted amino acid residue may or may not be one encoded by the genetic code. A variant of a polynucleotide or polypeptide may be a naturally occurring such as an allelic variant, or it may be a variant that is not known to occur naturally. Non-naturally occurring variants of polynucleotides and polypeptides may be made by mutagenesis techniques, by direct synthesis, and by other recombinant methods known to the persons skilled in the art.

In some embodiments, the disclosure contemplates making variants by modifying the structure of an enzyme as used in the disclosure. Variants can be produced by amino acid substitution, deletion, addition, or combinations thereof.

“Fragment,” with respect to a polynucleotide, refers to a clone or any part of a polynucleotide molecule, particularly a part of a polynucleotide.

Throughout the application, the sequence of a polynucleotide can be represented by a SEQ ID NO or alternatively by a GeneID (Maglott et al. (2011) Nucl. Acids Res. 39, Issue suppl_1, D52-D57) or GenBank NO (ncbi.nlm.nih.gov/genbank/). Therefore, the terms “polynucleotide SEQ ID NO,” “polynucleotide GeneID” and “polynucleotide GenBank NO.” can be interchangeably used, unless explicitly stated otherwise. Throughout the application, the sequence of a polypeptide can be represented by a SEQ ID NO or alternatively by an UniProt ID. Therefore, the terms “polypeptide SEQ ID NO” and “polypeptide UniProt ID” can be interchangeably used, unless explicitly stated otherwise.

A domain can be characterized, for example, by a Pfam (El-Gebali et al., Nucleic Acids Res. 47 (2019) D427-D432), an IPR (InterPro domain) (ebi.ac.uk/interpro) (Mitchell et al., Nucleic Acids Res. 47 (2019) D351-D360), a Conserved Domain Database (CDD) designation (ncbi.nlm.nih.gov/cdd) (Lu et al., Nucleic Acids Res. 48 (2020) D265-D268) or a PTHR domain (pantherdb.org) (Mi et al., Nucleic Acids. Res. 41 (2013) D377-D386; Thomas et al., Genome Research 13 (2003) 2129-2141). Protein or polypeptide sequence information and functional information can be provided by a comprehensive resource for protein sequence and annotation data like, e.g., the Universal Protein Resource (UniProt) (uniprot.org) (Nucleic Acids Res. 2021, 49(D1), D480-D489). UniProt comprises the expertly and richly curated protein database called the UniProt Knowledgebase (UniProtKB), together with the UniProt Reference Clusters (UniRef) and the UniProt Archive (UniParc). The UniProt identifiers (UniProt ID) are unique for each protein present in the database. Throughout the application, the sequence of a polypeptide is represented by a SEQ ID NO or an UniProt ID. Unless stated otherwise, the UniProt IDs of the proteins described correspond to their sequence version 01 as present in the UniProt Database version release 2021_03 and consulted on 9 Jun. 2021.

InterPro provides functional analysis of proteins by classifying them into families and predicting domains and important sites. To classify proteins in this way, InterPro uses predictive models, known as signatures, provided by several different databases (referred to as member databases) that make up the InterPro consortium. Protein signatures from these member databases are combined into a single searchable resource, capitalizing on their individual strengths to produce a powerful integrated database and diagnostic tool.

It should be understood for those skilled in the art that for the databases used herein, comprising InterPro 90.0 (released on 4 Aug. 2022) and eggNOG5.0 (released in 2019), the content of each database is fixed at each release and is not to be changed. When the content of a specific database is changed, this specific database receives a new release version with a new release date. All release versions for each database with their corresponding release dates and specific content as annotated at these specific release dates are available and known to those skilled in the art.

The terms “lactobionic acid,” “LBA,” “4-O-β-D-Galactopyranosyl-D-gluconic acid,” “4-O-b-galactopyranosyl-D-gluconic acid,” “4-(β-D-Galactosido)-D-gluconic acid,” “lactobionate,” “maltobionic-acid” and “(2R,3R,4R,5R)-2,3,5,6-tetrahydroxy-4-[(2S,3R,4S,5R,6R)-3,4,5-trihydroxy-6-(hydroxyrnethyl)oxan-2-yl]oxyhexanoic acid” are used interchangeably and refer to C₁₂H₂₂O₁₂, which is a disaccharide formed between β-D-galactose and D-gluconic acid.

The term “modified form of LBA,” as used herein, refers to a LBA molecule that is further modified with one or more other molecules like, e.g., with one or more monosaccharide(s), disaccharide(s), oligosaccharide(s), chitosan, chemical group(s) and/or amino group(s) or to a LBA molecule that is present in a complex with one or mineral(s), like, e.g., calcium, magnesium, potassium, sodium, iron, zinc, copper, chromium, selenium, manganese. An example of a modified form of LBA is a glycosylated form of LBA as described herein.

The terms “glucosylated form of LBA” and “glycosylated form of LBA,” as used herein, are used interchangeably and refer to a LBA molecule that is further modified with one or more monosaccharide(s). The monosaccharide(s) are further defined herein. Examples of a glycosylated form of LBA comprise fucosylated LBA and sialylated LBA. The term “fucosylated LBA,” as used herein, refers to a LBA molecule that is further modified with one or more fucose residue(s). Examples of a fucosylated LBA comprise O-6-deoxy-a-L-galactopyranosyl-(1-2)-O-b-D-galactopyranosyl-(1-4)-D-gluconic acid (2′FLBA, 2′-fucosylated LBA) and O-6-deoxy-a-L-galactopyranosyl-(1-3)-O-[b-D-galactopyranosyl-(1-4)-]-D-gluconic acid (3FLBA, 3-fucosylated LBA). The term “sialylated LBA,” as used herein, refers to a LBA molecule that is further modified with one or more sialic acid residue(s). Examples of a sialylated LBA comprise O—(N-acetyl-a-neuraminosyl)-(2-3)-O-b-D-galactopyranosyl-(1-4)-D-gluconic acid (3′SLBA, 3′-sialylated LBA) and O—(N-acetyl-a-neuraminosyl)-(2-6)-O-b-D-galactopyranosyl-(1-4)-D-gluconic acid (6'S-LBA, 6′-sialylated LBA).

The term “monosaccharide,” as used herein, refers to a sugar that is not decomposable into simpler sugars by hydrolysis, is classed as an aldose, a ketose, a deoxysugar, a deoxy-aminosugar, a uronic acid, an aldonic acid, a ketoaldonic acid, an aldaric acid or a sugar alcohol, and contains one or more hydroxyl groups per molecule. Monosaccharides are saccharides containing only one simple sugar.

The term “phosphorylated monosaccharide,” as used herein, refers to a monosaccharide that is phosphorylated. Examples of phosphorylated monosaccharides include but are not limited to glucose-1-phosphate, glucose-6-phosphate, glucose-1,6-bisphosphate, galactose-1-phosphate, fructose-6-phosphate, fructose-1,6-bisphosphate, fructose-1-phosphate, glucosamine-1-phosphate, glucosamine-6-phosphate, N-acetylglucosamine-1-phosphate, mannose-1-phosphate, mannose-6-phosphate or fucose-1-phosphate.

The terms “activated monosaccharide,” “nucleotide-activated sugar,” “nucleotide-sugar,” “activated sugar,” “nucleoside” or “nucleotide donor” are used herein interchangeably and refer to activated forms of monosaccharides. Examples of activated monosaccharides include but are not limited to UDP-N-acetylglucosamine (UDP-GlcNAc), UDP-N-acetylgalactosamine (UDP-GalNAc), UDP-N-acetylmannosamine (UDP-ManNAc), UDP-glucose (UDP-Glc), UDP-galactose (UDP-Gal), GDP-mannose (GDP-Man), UDP-glucuronate, UDP-galacturonate, UDP-2-acetamido-2,6-dideoxy-L-arabino-4-hexulose, UDP-2-acetamido-2,6-dideoxy-L-lyxo-4-hexulose, UDP-N-acetyl-L-rhamnosamine (UDP-L-RhaNAc or UDP-2-acetamido-2,6-dideoxy-L-mannose), dTDP-N-acetylfucosamine, UDP-N-acetylfucosamine (UDP-L-FucNAc or UDP-2-acetamido-2,6-dideoxy-L-galactose), UDP-N-acetyl-L-pneumosamine (UDP-L-PneNAC or UDP-2-acetamido-2,6-dideoxy-L-talose), UDP-N-acetylmuramic acid, UDP-N-acetyl-L-quinovosamine (UDP-L-QuiNAc orUDP-2-acetamido-2,6-dideoxy-L-glucose), GDP-L-quinovose, CMP-sialic acid (CMP-Neu5Ac or CMP-N-acetylneuraminic acid), GDP-fucose (GDP-Fuc), GDP-rhamnose and UDP-xylose. Nucleotide-sugars act as glycosyl donors in glycosylation reactions. Glycosylation reactions are reactions that are catalyzed by glycosyltransferases.

The term “glycosyltransferase,” as used herein, refers to an enzyme capable of catalyzing the transfer of a sugar moiety of a donor to a specific acceptor, forming glycosidic bonds. The donor can be a precursor as defined herein. A classification of glycosyltransferases using nucleotide diphospho-sugar, nucleotide monophospho-sugar and sugar phosphates and related proteins into distinct sequence-based families has been described (Campbell et al., Biochem. J 326, 929-939 (1997)) and is available on the CAZy (CArbohydrate-Active EnZymes) website (cazy.org). As used herein, the glycosyltransferase can be selected from the list comprising, consisting of or consisting essentially of: fucosyltransferases, sialyltransferases, galactosyltransferases, glucosyltransferases, mannosyltransferases, N-acetylglucosaminyltransferases, N-acetylgalactosaminyltransferases, N-acetylmannosaminyltransferases, xylosyltransferases, glucuronyltransferases, galacturonyltransferases, glucosaminyltransferases, N-glycolylneuraminyltransferases, rhamnosyltransferases, N-acetylrhamnosyltransferases, UDP-4-amino-4,6-dideoxy-N-acetyl-β-L-altrosamine transaminases, UDP-N-acetylglucosamine enolpyruvyl transferases and fucosaminyltransferases.

The term “disaccharide,” as used herein, refers to a saccharide polymer containing two simple sugars, i.e., monosaccharides. Examples of disaccharides comprise lactose (Gal-β1,4-Glc), lacto-N-biose (Gal-β1,3-GlcNAc), N-acetyllactosamine (Gal-β1,4-GlcNAc), LacDiNAc (GalNAc-β1,4-GlcNAc), N-acetylgalactosaminylglucose (GalNAc-β1,4-Glc), Neu5Ac-α2,3-Gal, Neu5Ac-α2,6-Gal and fucopyranosyl-(1-4)-N-glycolylneuraminic acid (Fuc-(1-4)-Neu5Gc), sucrose (Glc-α1,2-Fru), maltose (Glc-α1,4-Glc) and melibiose (Gal-α1,6-Glc).

“Oligosaccharide,” as the term is used herein and as generally understood in the state of the art, refers to a saccharide polymer containing a small number, typically three to twenty, preferably three to ten, of simple sugars, i.e., monosaccharides. The oligosaccharide as used in the disclosure can be a linear structure or can include branches. The linkage (e.g., glycosidic linkage, galactosidic linkage, glucosidic linkage, etc.) between two sugar units can be expressed, for example, as 1,4, 1->4, or (1-4), used interchangeably herein. For example, the terms “Gal-β1,4-Glc,” “Gal-β1,4-Glc,” “b-Gal-(1->4)-Glc,” “b-Gal-(1->4)-Glc,” “Galβ1-4-Glc,” “Gal-b(1-4)-Glc” and “Gal-b(1-4)-Glc” have the same meaning, i.e., a β-glycosidic bond links carbon-1 of galactose (Gal) with the carbon-4 of glucose (Glc). Each monosaccharide can be in the cyclic form (e.g., pyranose or furanose form). Linkages between the individual monosaccharide units may include α 1->2, α 1->3, α 1->4, α 1->6, α 2->1, α 2->3, α 2->4, α 2->6, β 1->2, β 1->3, β 1->4, β 1->6, β 2>1, β 2->3, β 2->4, and β 2->6. An oligosaccharide can contain both α- and β-glycosidic bonds or can contain only α-glycosidic or only β-glycosidic bonds. The term “polysaccharide” refers to a compound consisting of a large number, typically more than twenty, of monosaccharides linked glycosidically. Examples of oligosaccharides include but are not limited to Lewis-type antigen oligosaccharides, milk oligosaccharides, mammalian (including human) milk oligosaccharides, O-antigen, enterobacterial common antigen (ECA), the glycan chain present in lipopolysaccharides (LPS), the oligosaccharide repeats present in capsular polysaccharides, peptidoglycan (PG), amino-sugars, antigens of the human ABO blood group system, an animal oligosaccharide, preferably selected from the list consisting of N-glycans and O-glycans, a plant oligosaccharide, preferably selected from the list consisting of N-glycans and O-glycans, sialylated oligosaccharide, neutral (non-charged) oligosaccharide, negatively charged oligosaccharide, fucosylated oligosaccharide, N-acetylglucosamine containing oligosaccharides, lacto-N-biose containing oligosaccharides, N-acetyllactosamine-containing oligosaccharides, N-acetylglucosamine containing sialylated oligosaccharides, N-acetylglucosamine containing neutral (non-charged) oligosaccharides, N-acetylglucosamine containing negatively charged oligosaccharides, N-acetylglucosamine containing fucosylated oligosaccharides, N-acetylglucosamine containing non-fucosylated oligosaccharides, lacto-N-biose containing sialylated oligosaccharides, lacto-N-biose containing neutral (non-charged) oligosaccharides, lacto-N-biose containing negatively charged oligosaccharides, lacto-N-biose containing fucosylated oligosaccharides, lacto-N-biose containing non-fucosylated oligosaccharides, N-acetyllactosamine containing sialylated oligosaccharides, N-acetyllactosamine containing neutral (non-charged) oligosaccharides, N-acetyllactosamine containing negatively charged oligosaccharides, N-acetyllactosamine containing fucosylated oligosaccharides, N-acetyllactosamine containing non-fucosylated oligosaccharides, chitosan, chitosan comprising oligosaccharide, heparosan, chondroitin sulphate, glycosaminoglycan oligosaccharide, heparin, heparan sulphate, dermatan sulphate, hyaluronan, hyaluronic acid, keratan sulphate, erlose (Glc-α1,4-Glc-α1,2-Fru), lactul-N-triose II (GlcNAc-β1,3-Gal-β1,4-Fru), lactul-N-tetraose, lactul-N-neotetraose and globotriose.

The terms “negatively charged oligosaccharide” or “acidic oligosaccharide” are used interchangeably and refer to an oligosaccharide with a negative charge. In a preferred embodiment, the negatively charged oligosaccharide is a sialylated oligosaccharide. As used herein, a “sialylated oligosaccharide” is to be understood as a negatively charged sialic acid containing oligosaccharide, i.e., an oligosaccharide having one or more sialic acid residue(s). It has an acidic nature. Some examples are 3′SL (3′-sialyllactose, Neu5Ac-α2,3-Gal-β1,4-Glc), 3′-sialyllactosamine, 6′SL (6′sialyllactose, Neu5Ac-α2,6-Gal-β1,4-Glc), 8′SL (8′sialyllactose, Neu5Ac-α2,8-Gal-β1,4-Glc), 3,6-disialyllactose (Neu5Ac-α2,3-(Neu5Ac-α2,6)-Gal-β1,4-Glc), 6,6′-disialyllactose (Neu5Ac-α2,6-Gal-β1,4-(Neu5Ac-α2,6)-Glc), 8,3-disialyllactose (Neu5Ac-α2,8-Neu5Ac-α2,3-Gal-β1,4-Glc), 6′-sialyllactosamine, 3′-sialyllactosamine, oligosaccharides comprising 6′sialyllactose, SGG hexasaccharide (Neu5Aca-2,3Galβ-1,3GalNacβ-1,3Gala-1,4Galβ-1,4Gal), sialylated tetrasaccharide, sialylated pentasaccharide, sialylated lacto-N-triose, sialylated lacto-N-tetraose, sialyllacto-N-neotetraose, LSTc (Neu5Ac-α2,6-Gal-β1,4-GlcNAc-β1,3-Gal-β1,4-Glc), LSTd (Neu5Ac-α2,3-Gal-β1,4-GlcNAc-β1,3-Gal-β1,4-Glc), monosialyllacto-N-hexaose, disialyllacto-N-hexaose I, monosialyllacto-N-neohexaose I, monosialyllacto-N-neohexaose II, disialyllacto-N-neohexaose, disialyllacto-N-tetraose, disialyllacto-N-hexaose II, sialyllacto-N-tetraose a (LSTa, Neu5Ac-α2,3-Gal-β1,3-GlcNAc-β1,3-Gal-β1,4-Glc), disialyllacto-N-hexaose I, sialyllacto-N-tetraose b (LSTb, Gal-β1,3-(Neu5Ac-α2,6)-GlcNAc-β1,3-Gal-β1,4-Glc), 3′-sialyl-3-fucosyllactose, fucodisialyllacto-N-hexaose, disialomonofucosyllacto-N-neohexaose, monofucosylmonosialyllacto-N-octaose (sialyl Lea), sialyllacto-N-fucohexaose II, disialyllacto-N-fucopentaose II, monofucosyldisialyllacto-N-tetraose, Neu5Ac-α2,3-Gal-β1,4-GlcNAc-β1,3-Gal, Neu5Ac-α2,3-Gal-β1,3-GlcNAc-β1,3-Gal, 3′-KDO-lactose, 3′-KDO-lactosamine, 3′-KDO-6′sialyllactose, 3′KDO-8-sialyllactose, KDO-2,3Galβ-1,3GalNacβ-1,3Gala-1,4Galβ-1,4Gal, KDO-2,3Galβ-1,3GlcNacβ-1,3Galβ-1,4Glc, KDO-2,3Galβ-1,4GlcNacβ-1,3Galβ-1,4Glc, 3′-KDO-3-fucosyllactose, Neu5Ac-α2,8-Neu5Ac-α2,3-Gal-β1,3-GlcNAc-β1,3-Gal, 3′-Sialyl-2′-fucosyllactose, 6′-Sialyl-2′-fucosyllactose, 6′-Sialyl-3-fucosyllactose, 3′-Sialyl-3-fucosyllactose, Neu5Ac-α2,6-(Neu5Ac-α2,3-)Gal-β1,4-Glc, 3′-Sialyl-3-fucosyllactosamine, Fuc-α1,4-(Neu5Ac-α2,3-Gal-β1,3-)GlcNAc, 6′-Sialyllacto-N-biose, 3′-Sialyllacto-N-biose, Neu5Ac-α2,6-(GlcNAc-β1,3-)Gal-β1,4-Glc, Neu5Ac-α2,6-(Gal-β1,4-(Fuc-α1,3-)GlcNAc-β1,3-)Gal-β1,4-Glc, Neu5Ac-α2,3-Gal-β1,4-(Fuc-α1,3-)GlcNAc-β1,3-Gal-β1,4-Glc, Neu5Ac-α2,6-(Neu5Ac-α2,6-Gal-β1,4-(Fuc-α1,3-)GlcNAc-β1,3-)Gal-β1,4-Glc, Neu5Ac-α2,6-(Gal-β1,4-GlcNAc-β1,3-)Gal-β1,4-Glc, Neu5Ac-α2,6-(Gal-β1,3-GlcNAc-β1,3-)Gal-β1,4-(Fuc-α1,3-)Glc, Neu5Ac-α2,6-Gal-β1,3-GlcNAc-β1,3-Gal-β1,4-(Fuc-al1,3-)Glc, Neu5Ac-α2,3-Gal-β1,3-GlcNAc-β1,3-Gal-β1,4-(Fuc-α1,3-)Glc, Neu5Ac-α2,3-(Fuc-α1,2-)Gal-β1,3-GlcNAc-β1,3-Gal-β1,4-Glc, Neu5Ac-α2,6-(Fuc-α1,2-Gal-β1,3-GlcNAc-β1,3-)Gal-β1,4-Glc, Neu5Ac-α2,6-(Fuc-α1,2-)Gal-β1,3-GlcNAc-β1,3-Gal-β1,4-Glc, Fuc-α1,4-(Neu5Ac-α2,3-Gal-β1,3-)GlcNAc-β1,3-Gal-β1,4-Glc, Neu5Ac-α2,6-(Neu5Ac-α2,6-Gal-β1,3-GlcNAc-β1,3-)Gal-β1,4-(Fuc-α1,3-)Glc, Neu5Ac-α2,6-(Neu5Ac-α2,6-(Fuc-α1,2-)Gal-β1,3-GlcNAc-β1,3-)Gal-β1,4-Glc, Neu5Ac-α2,6-(Gal-β1,3-GlcNAc-β1,3-)Gal-β1,4-Glc, Neu5Ac-α2,6-Gal-β1,3-GlcNAc-β1,3-Gal-β1,4-Glc and oligosaccharides bearing one or several sialic acid residue(s), including but not limited to: oligosaccharide moieties of the gangliosides selected from GM3 (3′sialyllactose, Neu5Aca-2,3Galβ-4Glc) and oligosaccharides comprising the GM3 motif, GD3 Neu5Aca-2,8Neu5Aca-2,3Galβ-1,4Glc GT3 (Neu5Aca-2,8Neu5Aca-2,8Neu5Aca-2,3Gal0-1,4Glc); GM2 GalNAcβ-1,4(Neu5Aca-2,3)Galβ-1,4Glc, GM1 Galβ-1,3GalNAcβ-1,4(Neu5Aca-2,3)Galβ-1,4Glc, GD1a Neu5Aca-2,3Galβ-1,3GalNAcβ-1,4(Neu5Aca-2,3)Galβ-1,4Glc, GT1a Neu5Acα-2,8Neu5Acα-2,3Galβ-1,3GaNAcβ-1,4(Neu5Acα-2,3)Galβ-1,4Glc, GD2 GalNAcβ-1,4(Neu5Acα-2,8Neu5Acα2,3)Galβ-1,4Glc, GT2 GalNAcβ-1,4(Neu5Acα-2,8Neu5Acα-2,8Neu5Acα2,3)Galβ-1,4Glc, GD1b, Galβ-1,3GalNAcβ-1,4(Neu5Acα-2,8Neu5Acα2,3)Galβ-1,4Glc, GT1b Neu5Acα-2,3Galβ-1,3GalNAcβ-1,4(Neu5Acα-2,8Neu5Acα2,3)Galβ-1,4Glc, GQ1b Neu5Acα-2,8Neu5Acα-2,3Galβ-1,3GalNAc β-1,4(Neu5Acα-2,8Neu5Acα2,3)Galβ-1,4Glc, GT1c Galβ-1,3GalNAcβ-1,4(Neu5Acα-2,8Neu5Acα-2,8Neu5Acα2,3)Galβ-1,4Glc, GQ1c Neu5Acα-2,3Galβ-1,3GalNAc β-1,4(Neu5Acα-2,8Neu5Acα-2,8Neu5Acα2,3)Galβ-1,4Glc, GP1c Neu5Acα-2,8Neu5Acα-2,3Galβ-1,3GalNAc β-1,4(Neu5Acα-2,8Neu5Acα-2,8Neu5Acα2,3)Galβ-1,4Glc, GD1a Neu5Acα-2,3Galβ-1,3(Neu5Acα-2,6)GalNAcβ-1,4Galβ-1,4Glc, Fucosyl-GM1 Fucα-1,2Galβ-1,3GalNAcβ-1,4(Neu5Acα-2,3)Gal β-1,4Glc; all of which may be extended to the production of the corresponding gangliosides by reacting the above oligosaccharide moieties with ceramide or synthetizing the above oligosaccharides on a ceramide.

“Charged oligosaccharides” are oligosaccharide structures that contain one or more negatively charged monosaccharide subunits including N-acetylneuraminic acid (Neu5Ac), commonly known as sialic acid, N-glycolylneuraminic acid (Neu5Gc), glucuronate, galacturonate and 2-keto-3-deoxymanno-octulonic acid (KDO). Charged oligosaccharides are also referred to as acidic oligosaccharides. In contrast, neutral (non-charged) oligosaccharides are non-sialylated oligosaccharides, and thus do not contain an acidic monosaccharide subunit. Neutral oligosaccharides comprise non-charged fucosylated oligosaccharides that contain one or more fucose subunits in their glycan structure as well as non-charged non-fucosylated oligosaccharides that lack any fucose subunit. Other examples of charged oligosaccharides are sulphated chitosans and deacetylated chitosans.

The terms “neutral oligosaccharide” and “non-charged” oligosaccharide, as used herein, are used interchangeably and refer, as generally understood in the state of the art, to an oligosaccharide that has no negative charge originating from a carboxylic acid group. Examples of such neutral oligosaccharide are 2′-fucosyllactose (2′FL), 3-fucosyllactose (3FL), 4-fucosyllactose (4FL), 6-fucosyllactose (6FL), 2′,3-difucosyllactose (diFL), lacto-N-triose II (LN3, GlcNAcβ1-3Galβ1-4Glc), lacto-N-tetraose (LNT, Galβ 1-3GlcNAcβ1-3Galβ 1-4Glc), lacto-N-neotetraose (LNnT, Galβ1-4GlcNAcβ1-3Galβ1-4Glc), lacto-N-fucopentaose I, lacto-N-neofucopentaose I, lacto-N-fucopentaose II, lacto-N-fucopentaose III, lacto-N-fucopentaose V, lacto-N-fucopentaose VI, lacto-N-neofucopentaose V, lacto-N-difucohexaose I, lacto-N-difucohexaose II, Fuc-α1,2-Gal-β1,3-GlcNAc-β1,3-Gal-β1,4-(Fuc-α1,3-)Glc, Fuc-α1,2-Gal-β1,4-GlcNAc-β1,3-Gal-β1,4-(Fuc-α1,3-)Glc, Fuc-α1,2-Gal-β1,4-(Fuc-α1,3-)GlcNAc-β1,3-Gal-β1,4-Glc, Gal-β1,4-(Fuc-α1,3-)GlcNAc-β1,3-Gal-β1,4-(Fuc-α1,3-)Glc, Fuc-α1,2-Gal-β1,4-(Fuc-α1,3-)GlcNAc-β1,3-Gal-β1,4-(Fuc-α1,3-)Glc, Fuc-α1,4-(Fuc-α1,2-Gal-β1,3-)GlcNAc-β1,3-Gal-β1,4-(Fuc-α1,3-)Glc, monofucosyllacto-N-hexaose-III, difucosyllacto-N-hexaose (a), 6′-galactosyllactose, 3′-galactosyllactose, lacto-N-hexaose, lacto-N-neohexaose, para-lacto-N-hexaose, para-lacto-N-neohexaose, difucosyl-lacto-N-hexaose, difucosyl-lacto-N-neohexaose, trifucosyllacto-N-hexaose, α1,3-galactosyl-3-fucosyllactose, Gal-α1,3-(Fuc-α1,2-)Gal-β1,4-(Fuc-α1,3-)Glc, GalNAc-α1,3-(Fuc-α1,2-)Gal-β1,4-(Fuc-α1,3-)Glc, 2-fucosyllactulose, 3-fucosyl-N-acetyllactosamine, 2′-fucosyl-N-acetyllactosamine, difucosyl-N-acetyllactosamine, 4-fucosyllacto-N-biose, 2′-fucosyllacto-N-biose, difucosyllacto-N-biose and GlcNAc-β1,3-Gal-β1,4-(Fuc-α1,3-)Glc, GlcNAc-β1,6-(GlcNAc-β1,3-)Gal-β1,4-Glc, lacto-N-pentaose (LN5), lacto-N-neopentaose, para lacto-N-pentaose, para lacto-N-neopentaose, lacto-N-novopentaose I, lacto-N-heptaose (LN7), lacto-N-neoheptaose, para lacto-N-neoheptaose, para lacto-N-heptaose, lacto-N-octaose (LNO), lacto-N-neooctaose, iso lacto-N-octaose, para lacto-N-octaose, iso lacto-N-neooctaose, novo lacto-N-neooctaose, para lacto-N-neooctaose (pLNnO), iso lacto-N-nonaose, novo lacto-N-nonaose, lacto-N-nonaose (LN9), lacto-N-decaose, iso lacto-N-decaose, novo lacto-N-decaose, lacto-N-neodecaose, para lacto-N-neodecaose (pLNnD), α1,3-galactosyllacto-N-neotetraose, GlcNAc-β1,3-Gal-β1,3-GlcNAc-β1,3-Gal-β1,4-Glc, GlcNAc-β1,6-(Gal-β1,4-GlcNAc-β1,3-)Gal-β1,4-Glc and GlcNAc-β1,6-(Gal-β1,3-GlcNAc-β1,3-)Gal-β1,4-Glc.

A “fucosylated oligosaccharide,” as used herein and as generally understood in the state of the art, is an oligosaccharide that is carrying a fucose-residue. Such fucosylated oligosaccharide is a saccharide structure comprising at least three monosaccharide subunits linked to each other via glycosidic bonds, wherein at least one of the monosaccharide subunits is a fucose. A fucosylated oligosaccharide can contain more than one fucose residue, e.g., two, three or more. A fucosylated oligosaccharide can be a neutral oligosaccharide or a charged oligosaccharide, e.g., also comprising sialic acid structures. Fucose can be linked to other monosaccharide subunits comprising glucose, galactose, GlcNAc via α-glycosidic bonds comprising α-1,2 α-1,3, α-1,4, α-1,6 linkages. Examples comprise 2′-fucosyllactose (2′FL), 3-fucosyllactose (3FL), 4-fucosyllactose (4FL), 6-fucosyllactose (6FL), difucosyllactose (diFL), Lacto-N-fucopentaose I (LNFP I), Gal-α1,3-(Fuc-α1,2-)Gal-β1,3-GlcNAc-β1,3-Gal-β1,4-Glc (Gal-LNFP I), GalNAc-α1,3-(Fuc-α1,2-)Gal-β1,3-GlcNAc-β1,3-Gal-β1,4-Glc (GalNAc-LNFP I), Lacto-N-fucopentaose II (LNFP II), Lacto-N-fucopentaose III (LNFP III), lacto-N-fucopentaose V (LNFP V), lacto-N-fucopentaose VI (LNFP VI), lacto-N-neofucopentaose I, lacto-N-difucohexaose I (LDFH I), lacto-N-difucohexaose II (LDFH II), Monofucosyllacto-N-hexaose III (MFLNH III), difucosyllacto-N-hexaose (a), Difucosyllacto-N-hexaose, difucosyl-lacto-N-neohexaose, trifucosyllacto-N-hexaose, α1,3-galactosyl-3-fucosyllactose, Gal-α1,3-(Fuc-α1,2-)Gal-β1,4-(Fuc-α1,3-)Glc, GalNAc-α1,3-(Fuc-α1,2-)Gal-β1,4-(Fuc-α1,3-)Glc, 2-fucosyllactulose, 3-fucosyl-N-acetyllactosamine, 2′-fucosyl-N-acetyllactosamine, difucosyl-N-acetyllactosamine, 4-fucosyllacto-N-biose, 2′-fucosyllacto-N-biose, difucosyllacto-N-biose and GlcNAc-β1,3-Gal-β1,4-(Fuc-α1,3-)Glc, 3′-Sialyl-2′-fucosyllactose, 6′-Sialyl-2′-fucosyllactose, 6′-Sialyl-3-fucosyllactose, 3′-sialyl-3-fucosyllactose, disialomonofucosyllacto-N-neohexaose, monofucosylmonosialyllacto-N-octaose (sialyl Lea), sialyllacto-N-fucohexaose II, disialyllacto-N-fucopentaose II, monofucosyldisialyllacto-N-tetraose.

Mammalian milk oligosaccharides comprise oligosaccharides present in milk found in any phase during lactation including colostrum milk from humans and mammals. As used herein, “mammalian milk oligosaccharide” or “MMO” refers to oligosaccharides such as but not limited to 3-fucosyllactose, 2′-fucosyllactose, 6-fucosyllactose, 2′,3-difucosyllactose, 2′,2-difucosyllactose, 3,4-difucosyllactose, 6′-sialyllactose, 3′-sialyllactose, 3,6-disialyllactose, 6,6′-disialyllactose, 8,3-disialyllactose, 3,6-disialyllacto-N-tetraose, lacto-N-tetraose, lacto-N-neotetraose, lacto-N-fucopentaose II, lacto-N-fucopentaose I, lacto-N-fucopentaose III, lacto-N-fucopentaose V, lacto-N-fucopentaose VI, sialyllacto-N-tetraose c, sialyllacto-N-tetraose b, sialyllacto-N-tetraose a, lacto-N-difucohexaose I, lacto-N-difucohexaose II, lacto-N-hexaose, lacto-N-neohexaose, para-lacto-N-hexaose, monofucosylmonosialyllacto-N-tetraose c, monofucosyl para-lacto-N-hexaose, monofucosyllacto-N-hexaose III, isomeric fucosylated lacto-N-hexaose III, isomeric fucosylated lacto-N-hexaose I, sialyllacto-N-hexaose, sialyllacto-N-neohexaose II, difucosyl-para-lacto-N-hexaose, difucosyllacto-N-hexaose, difucosyllacto-N-hexaose a, difucosyllacto-N-hexaose c, galactosylated chitosan, fucosylated oligosaccharides, neutral oligosaccharides and/or sialylated oligosaccharides.

The terms “human milk oligosaccharide” or “HMO” refer to oligosaccharides found in human breast milk, including preterm human milk, colostrum and term human milk. HMOs comprise fucosylated oligosaccharides, non-fucosylated neutral oligosaccharides and sialylated oligosaccharides. Examples of HMOs comprise 3-fucosyllactose, 2′-fucosyllactose, 2′,3-difucosyllactose, 6′-sialyllactose, 3′-sialyllactose, LN3, lacto-N-tetraose, lacto-N-neotetraose, lacto-N-fucopentaose II, lacto-N-fucopentaose I, lacto-N-fucopentaose III, lacto-N-fucopentaose V, lacto-N-fucopentaose VI, sialyllacto-N-tetraose c, sialyllacto-N-tetraose b, sialyllacto-N-tetraose a, difucosyllacto-N-tetraose, lacto-N-hexaose, lacto-N-difucohexaose I, lacto-N-difucohexaose II, disialyllacto-N-tetraose, fucosyllacto-N-hexaose, difucosyllacto-N-hexaose, fucodisialyllacto-N-hexaose, disialyllacto-N-hexaose.

The term “pathway for production of a saccharide” as used herein is a biochemical pathway consisting of the enzymes and their respective genes involved in the synthesis of a saccharide as defined herein. The pathway for production of a saccharide can comprise but is not limited to pathways involved in the synthesis of a nucleotide-activated sugar and the transfer of the nucleotide-activated sugar to an acceptor to create a saccharide of the disclosure. Examples of such pathways comprise but are not limited to a fucosylation pathway, a sialylation pathway, a galactosylation pathway, an N-acetylglucosaminylation pathway, an N-acetylgalactosaminylation pathway, a mannosylation pathway and an N-acetylmannosaminylation pathway.

The terms “LBA synthesis” or “pathway for production of LBA” as used herein are used interchangeably and refer to a biochemical pathway consisting of the enzymes and their respective genes involved in the synthesis of lactobionic acid (LBA). The pathway for production of LBA may comprise a pathway for synthesis and/or import of a co-factor used in the pathway for production of LBA.

The terms “synthesis of a modified form of LBA, a glycosylated form of LBA, fucosylated LBA and/or sialylated LBA” or “pathway for production of a modified form of LBA, a glycosylated form of LBA, fucosylated LBA and/or sialylated LBA” as used herein are used interchangeably and refer to a biochemical pathway consisting of the enzymes and their respective genes involved in the synthesis of a modified form of LBA, a glycosylated form of LBA, fucosylated LBA and/or sialylated LBA, respectively. The pathway for production of a modified form of LBA, a glycosylated form of LBA, fucosylated LBA and/or sialylated LBA may comprise a pathway for synthesis and/or import of a co-factor used in the pathway for production of a modified form of LBA, a glycosylated form of LBA, fucosylated LBA and/or sialylated LBA, respectively.

The term “purified” refers to material that is substantially or essentially free from components that interfere with the activity of the biological molecule. For cells, saccharides, nucleic acids, and polypeptides, the term “purified” refers to material that is substantially or essentially free from components that normally accompany the material as found in its native state. Typically, purified saccharides, oligosaccharides, proteins or nucleic acids of the disclosure are at least about 50%, 55%, 60%, 65%, 70%, 75%, 80% or 85% pure, usually at least about 90%, 91%, 92%, 93%, 94%, 95%, 96%, 97%, 98%, or 99.0% pure as measured by band intensity on a silver-stained gel or other method for determining purity. Purity or homogeneity can be indicated by a number of means well known in the art, such as polyacrylamide gel electrophoresis of a protein or nucleic acid sample, followed by visualization upon staining. For certain purposes high resolution will be needed and HPLC or a similar means for purification utilized. For di- and oligosaccharides, purity can be determined using methods such as, but not limited to, thin layer chromatography, gas chromatography, NMR, HPLC, capillary electrophoresis or mass spectroscopy. Further herein, the terms “contaminants” and “impurities” preferably mean particulates, cells, cell components, metabolites, cell debris, proteins, peptides, amino acids, nucleic acids, glycolipids and/or endotoxins that can be present in an aqueous medium like, e.g., a cultivation or an incubation.

The term “clarifying” as used herein refers to the act of treating an aqueous medium like, e.g., a cultivation, an incubation, to remove suspended particulates and contaminants from the production process like, e.g., cells, cell components, insoluble metabolites and debris, that could interfere with the eventual purification of the saccharide. Such treatment can be carried out in a conventional manner by centrifugation, flocculation, flocculation with optional ultrasonic treatment, gravity filtration, microfiltration, foam separation or vacuum filtration (e.g., through a ceramic filter, which can include a Celite™ filter aid).

The term “cultivation” refers to the culture medium wherein the cell is cultivated, or fermented, the cell itself, and a saccharide, LBA, a modified form of LBA, a glycosylated form of LBA, fucosylated LBA and/or sialylated LBA that is produced by the cell in whole broth, i.e., inside (intracellularly) as well as outside (extracellularly) of the cell. The terms “culture medium” and “cultivation medium” as used herein are used interchangeably and refer to the medium wherein the cell is cultivated.

The term “incubation” refers to a mixture wherein i) a saccharide or ii) a saccharide, LBA and/or a modified form of LBA, a glycosylated form of LBA, fucosylated LBA and/or sialylated LBA is produced. The mixture can comprise one or more enzyme(s), one or more precursor(s) and one or more acceptor(s) as defined herein present in a buffered solution and incubated for a certain time at a certain temperature enabling production of a saccharide, catalyzed by one or more enzyme(s) using the one or more precursor(s) and the one or more acceptor(s) in the mixture. The mixture can also comprise i) the cell obtained after cultivation or incubation, optionally the cell is subjected to cell lysis, ii) a buffered solution or the cultivation or incubation medium wherein the cell was cultivated or fermented, and iii) a) a saccharide or b) a saccharide, LBA and/or a modified form of LBA, a glycosylated form of LBA, fucosylated LBA and/or sialylated LBA that is/are produced by the cell in whole broth, i.e., inside (intracellularly) as well as outside (extracellularly) of the cell. The incubation can also be the cultivation as defined herein.

The terms “reactor” and “incubator” refer to the recipient filled with the cultivation or incubation. Examples of reactors and incubators comprise but are not limited to microfluidic devices, well plates, tubes, shake flasks, fermenters, bioreactors, process vessels, cell culture incubators, CO₂ incubators.

As used herein, the term “cell productivity index (CPI)” refers to the mass of the saccharide produced by the cells divided by the mass of the cells produced in the culture. The term “CPI” as used herein is also to be understood as mass of the LBA produced by the cells divided by the mass of the cells produced in the culture. Further, the term “CPI” as used herein is also to be understood as mass of the saccharide and the LBA produced by the cells divided by the mass of the cells produced in the culture. Also, the term “CPI” as used herein is to be understood as mass of a modified form, a glycosylated form of LBA, fucosylated LBA and/or sialylated LBA produced by the cells divided by the mass of the cells produced in the culture. Also, the term “CPI” as used herein is to be understood as mass of saccharide, LBA and a modified form of LBA, a glycosylated form of LBA, fucosylated LBA and/or sialylated LBA produced by the cells divided by the mass of the cells produced in the culture.

The term “precursor” as used herein refers to substances that are taken up or synthetized by the cell for the specific production of i) a saccharide, ii) LBA and/or iii) a saccharide, LBA, a modified form of LBA, a glycosylated form of LBA, fucosylated LBA and/or sialylated LBA according to the disclosure. In this sense, a precursor can be an acceptor as defined herein, but can also be another substance, metabolite, a co-factor that is first modified within the cell as part of the biochemical synthesis route of i) a saccharide, ii) LBA and/or iii) a saccharide, LBA, a modified form of LBA, a glycosylated form of LBA, fucosylated LBA and/or sialylated LBA. The term “precursor” as used herein is also to be understood as a chemical compound that participates in an incubation or an enzymatic reaction to produce another compound like, e.g., an intermediate or an acceptor as defined herein, as part in the metabolic pathway of i) a saccharide, ii) LBA and/or iii) a saccharide, LBA, a modified form of LBA, a glycosylated form of LBA, fucosylated LBA and/or sialylated LBA. The term “precursor” as used herein is also to be understood as a donor that is used by a glycosyltransferase to modify an acceptor as defined herein with a sugar moiety in a glycosidic bond, as part in the metabolic pathway of a saccharide. Examples of such precursors comprise the acceptors as defined herein, and/or dihydroxyacetone, glucosamine, N-acetylglucosamine, N-acetylmannosamine, galactosamine, N-acetylgalactosamine, galactosyllactose, phosphorylated sugars or sugar phosphates like, e.g., but not limited to, glucose-1-phosphate, galactose-1-phosphate, glucose-6-phosphate, fructose-6-phosphate, fructose-1,6-bisphosphate, mannose-6-phosphate, mannose-1-phosphate, glycerol-3-phosphate, glyceraldehyde-3-phosphate, dihydroxyacetone-phosphate, glucosamine-6-phosphate, N-acetylglucosamine-6-phosphate, N-acetylmannosamine-6-phosphate, N-acetylglucosamine-1-phosphate, N-acetylneuraminic acid-9-phosphate and nucleotide-activated sugars like nucleotide diphospho-sugars and nucleotide monophospho-sugars as defined herein like, e.g., UDP-glucose, UDP-galactose, UDP-N-acetylglucosamine, CMP-sialic acid, GDP-mannose, GDP-4-dehydro-6-deoxy-α-D-mannose, GDP-fucose.

Optionally, the cell used to produce the saccharide is transformed to comprise and to express at least one nucleic acid sequence encoding a protein selected from the group consisting of lactose transporter, N-acetylneuraminic acid transporter, fucose transporter, glucose transporter, galactose transporter, transporter for a nucleotide-activated sugar wherein the transporter internalizes a to the medium added precursor for the synthesis of the saccharide of the disclosure.

The term “acceptor” as used herein refers to a mono-, di- or oligosaccharide, which can be modified by a glycosyltransferase. Examples of such acceptors comprise glucose, galactose, fructose, glycerol, sialic acid, fucose, mannose, maltose, sucrose, lactose, lactulose, lactobionic acid (LBA), lacto-N-triose, lacto-N-tetraose (LNT), lacto-N-neotetraose (LNnT), lacto-N-pentaose (LNP), lacto-N-neopentaose, para lacto-N-pentaose, para lacto-N-neopentaose, lacto-N-novopentaose I, lacto-N-hexaose (LNH), lacto-N-neohexaose (LNnH), para lacto-N-neohexaose (pLNnH), para lacto-N-hexaose (pLNH), lacto-N-heptaose, lacto-N-neoheptaose, para lacto-N-neoheptaose, para lacto-N-heptaose, lacto-N-octaose (LNO), lacto-N-neooctaose, iso lacto-N-octaose, para lacto-N-octaose, iso lacto-N-neooctaose, novo lacto-N-neooctaose, para lacto-N-neooctaose, iso lacto-N-nonaose, novo lacto-N-nonaose, lacto-N-nonaose, lacto-N-decaose, iso lacto-N-decaose, novo lacto-N-decaose, lacto-N-neodecaose, and oligosaccharide containing 1 or more N-acetyllactosamine units and/or 1 or more lacto-N-biose units or an intermediate into oligosaccharide, fucosylated and sialylated versions thereof, ceramide, N-acylated sphingoid, glucosylceramide, lactosylceramide, sphingosine, phytosphingosine, sphingosine synthons, peptide backbones with β-GlcNAc-Asn residues, glycoproteins with terminal GlcNAc and Gal residues, immunoglobulins.

### BRIEF DESCRIPTION OF THE DRAWING

FIG. 1: Growth speed in relative percentages (%) obtained in a growth experiment with modified E. coli strains engineered for production of 2′FL, LNT, LNnT, 3′SL or 6′SL as described in Example 1 and having native gcd and ylil expression (reference strain, gcd+ylil+) or having a gcd genomic knock-out (gcd KO, ylil+) or having a gcd genomic knock-out and a ylil genomic knock-out (gcd KO, ylil KO). The growth experiment was performed according to the culture conditions provided in Example 1, in which the culture or cultivation medium was supplemented with 30 g/L sucrose, 20 g/L lactose and 3.30E-4 g/L pyrroloquinoline quinone (PQQ). The dashed horizontal line indicates the setpoint to which all adaptations were normalized.

### DETAILED DESCRIPTION

According to a first aspect, the disclosure provides a cell i) capable of synthesizing and/or synthesizing lactobionic acid (4-O-b-galactopyranosyl-D-gluconic acid, LBA), a modified form of LBA, a glycosylated form of LBA, fucosylated LBA and/or sialylated LBA in the presence of lactose in the cultivation or incubation medium of the cell and ii) genetically engineered for the production of a saccharide, wherein the cell comprises a pathway for production of the saccharide, characterized in that the synthesis of the LBA, modified form of LBA, glycosylated form of LBA, fucosylated LBA and/or sialylated LBA in the cell is rendered less functional or is knocked out. Herein, synthesis of the LBA, modified form of LBA, glycosylated form of LBA, fucosylated LBA and/or sialylated LBA is obtained by expression of at least one gene selected from the list consisting of genes encoding carbohydrate oxidase, dehydrogenase, lactonase, oxygen-dependent FAD-linked oxidoreductase, and pyrroloquinoline quinone (PQQ) oxidoreductase, wherein the synthesis of the LBA, modified form of LBA, glycosylated form of LBA, fucosylated LBA and/or sialylated LBA in the cell is rendered less functional or is knocked out by rendering less functional or knock out of the at least one gene, two or more of the genes or all of the genes.

According to a second aspect, the disclosure provides a method for the production of a saccharide, wherein the method comprises cultivating and/or incubating a cell as described herein, in cultivation and/or incubation medium under conditions permissive to produce a saccharide and LBA.

In the scope of the disclosure, permissive conditions are understood to be conditions relating to physical or chemical parameters including but not limited to temperature, pH, pressure, osmotic pressure and product/precursor/acceptor/co-factor concentration. It is to be understood herein that the conditions comprise the presence of lactose in the cultivation or incubation medium wherein the cell is cultivated and/or incubated.

In a particular embodiment, the permissive conditions may include a temperature-range of 30+/−20 degrees centigrade, a pH-range of 7+/−3.

In a preferred embodiment, the saccharide is separated from the cultivation and/or incubation. In another and/or additional preferred embodiment, the saccharide is separated from LBA modified form of LBA, glycosylated form of LBA, fucosylated LBA and/or sialylated LBA. In another and/or additional preferred embodiment, the saccharide is purified.

In a specific embodiment of the method and/or cell of the disclosure, the cell is capable of synthesizing LBA, a modified form of LBA, a glycosylated form of LBA, fucosylated LBA and/or sialylated LBA. LBA can be produced by oxidation of lactose due to a dehydrogenase (lactose-oxidase) system into lactone, which is further hydrolyzed into LBA. LBA can also be produced upon oxidation of maltose. LBA can also be produced by lactose dehydrogenase and lactonase. LBA can also be formed out of other saccharides than lactose like, e.g., but not limited to, D-glucose, D-galactose, D-mannose, D-talose, D-xylose, D-ribose, L-arabinose, cellobiose and D-fructose.

In a preferred embodiment of the method and/or cell, the cell synthesizes LBA, a modified form of LBA, a glycosylated form of LBA, fucosylated LBA and/or sialylated LBA. In a more preferred embodiment, the cell comprises a pathway for production of LBA, a modified form of LBA, a glycosylated form of LBA, fucosylated LBA and/or sialylated LBA. The pathway for production of LBA, a modified form of LBA, a glycosylated form of LBA, fucosylated LBA and/or sialylated LBA consists of the enzymes and their respective genes involved in the synthesis of LBA, the modified form of LBA, the glycosylated form of LBA, fucosylated LBA and/or sialylated LBA. Enzymes involved in the synthesis of LBA, a modified form of LBA, a glycosylated form of LBA, fucosylated LBA and/or sialylated LBA comprise but are not limited to carbohydrate oxidase, dehydrogenase, lactonase, oxygen-dependent FAD-linked oxidoreductase, pyrroloquinoline quinone (PQQ) oxidoreductase, lactose dehydrogenase, quinoprotein glucose dehydrogenase, aldose sugar dehydrogenase, glucose/fructose dehydrogenase, glucose/sorbosone dehydrogenase, cellobiose dehydrogenase, pyrroloquinoline quinone (PQQ) dehydrogenase, malate dehydrogenase and lactose oxidase. Synthesis of LBA, a modified form of LBA, a glycosylated form of LBA, fucosylated LBA and/or sialylated LBA may make use of one or more co-factor(s). Examples of co-factors comprise but are not limited to pyrroloquinoline quinone (PQQ), FAD, a divalent metal cation, Ca²⁺, Cu²⁺, Mg²⁺, Zn²⁺, heme, heme C and pyridoxal 5′-phosphate. The pathway for production of LBA, a modified form of LBA, a glycosylated form of LBA, fucosylated LBA and/or sialylated LBA may comprise a pathway for synthesis and/or for import of a co-factor used in a pathway for production of LBA, a modified form of LBA, a glycosylated form of LBA, fucosylated LBA and/or sialylated LBA. In other words, the cell of the disclosure may comprise a pathway for the production and/or import of any one or more of PQQ, FAD, a divalent metal cation, Ca²⁺, Cu²⁺, Mg²⁺, Zn²⁺, heme, heme C and pyridoxal 5′-phosphate. Alternatively, the cell of the disclosure does not synthesize a co-factor that is necessary in the synthesis of LBA, a modified form of LBA, a glycosylated form of LBA, fucosylated LBA and/or sialylated LBA but has all other enzymes necessary for synthesis of LBA, a modified form of LBA, a glycosylated form of LBA, fucosylated LBA and/or sialylated LBA. The cell may synthesize LBA, a modified form of LBA, a glycosylated form of LBA, fucosylated LBA and/or sialylated LBA upon supplementation with one or more co-factor(s).

In an additional specific embodiment, synthesis of LBA, a modified form of LBA, a glycosylated form of LBA, fucosylated LBA and/or sialylated LBA in the cell is rendered less functional or knocked out. Herein, a cell wherein synthesis of LBA, a modified form of LBA, a glycosylated form of LBA, fucosylated LBA and/or sialylated LBA is rendered less functional is to be understood as that the cell has lower production of LBA, a modified form of LBA, a glycosylated form of LBA, fucosylated LBA and/or sialylated LBA compared to a cell wherein synthesis, a modified form of LBA, a glycosylated form of LBA, fucosylated LBA and/or sialylated LBA, respectively, is not rendered less functional. According to the disclosure, synthesis of LBA, a modified form of LBA, a glycosylated form of LBA, fucosylated LBA and/or sialylated LBA is rendered less functional in a cell by making one or more genes involved in the pathway for production of LBA, a modified form of LBA, a glycosylated form of LBA, fucosylated LBA and/or sialylated LBA less functional. Alternatively, and/or additionally, synthesis of LBA, a modified form of LBA, a glycosylated form of LBA, fucosylated LBA and/or sialylated LBA is rendered less functional in a cell by making one or more genes involved in the pathway for production and/or the import of one or more co-factor(s) that are used in the synthesis of LBA, a modified form of LBA, a glycosylated form of LBA, fucosylated LBA and/or sialylated LBA less functional. Preferably, synthesis of LBA, a modified form of LBA, a glycosylated form of LBA, fucosylated LBA and/or sialylated LBA is rendered less functional in a cell by making one or more genes involved in the pathway for production and/or the import of one or more co-factor(s) selected from the list comprising, consisting of or consisting essentially of pyrroloquinoline quinone (PQQ), FAD, a divalent metal cation, Ca²⁺, Cu²⁺, Mg²⁺, Zn²⁺, heme, heme C and pyridoxal 5′-phosphate less functional. Rendering a gene less functional is to be understood as rendering a gene less able, i.e., statistically significantly “less able” compared to a functional wild-type gene or completely unable (such as knocked-out genes) to produce a functional final product. A gene can be made less functional by means of common well-known technologies for a skilled person by, e.g., any one or more of insertion, deletion and/or modification of one or more nucleotide(s) in one or more polynucleotide sequence(s) selected from the list comprising, consisting of or consisting essentially of promoter sequence, ribosome binding site, untranslated region, coding sequence and transcription terminator sequence of the gene so that the gene is made less able to produce a functional final product. Methods like, e.g., siRNA, CrispR, CrispRi, riboswitch, recombineering, homologous recombination, ssDNA mutagenesis, RNAi, miRNA, asRNA, mutating genes and transposon mutagenesis could be used herein. Alternatively, and/or additionally, synthesis of LBA, a modified form of LBA, a glycosylated form of LBA, fucosylated LBA and/or sialylated LBA is rendered less functional in a cell by replacement of the native pathway for production of LBA, a modified form of LBA, a glycosylated form of LBA, fucosylated LBA and/or sialylated LBA that is present in a cell by another pathway for production of LBA, a modified form of LBA, a glycosylated form of LBA, fucosylated LBA and/or sialylated LBA that gives lower production of LBA, a modified form of LBA, a glycosylated form of LBA, fucosylated LBA and/or sialylated LBA compared to the cell's native production pathway of LBA, a modified form of LBA, a glycosylated form of LBA, fucosylated LBA and/or sialylated LBA. A cell wherein synthesis of LBA, a modified form of LBA, a glycosylated form of LBA, fucosylated LBA and/or sialylated LBA is knocked out is to be understood that the cell does not produce LBA, a modified form of LBA, a glycosylated form of LBA, fucosylated LBA and/or sialylated LBA. A knock-out of a pathway in a cell can be made by genomic knock-out of one or more gene(s) involved in the pathway or making one or more gene(s) involved in the pathway unable to make a functional final product. A knock-out of a gene from the cell's genome can be made by methods well-known by a person skilled in the art.

In a preferred embodiment of the method and/or cell of the disclosure, synthesis of LBA, a modified form of LBA, a glycosylated form of LBA, fucosylated LBA and/or sialylated LBA is obtained by expression of at least one gene selected from the list consisting of genes encoding carbohydrate oxidase, dehydrogenase, lactonase, oxygen-dependent FAD-linked oxidoreductase, and pyrroloquinoline quinone (PQQ) oxidoreductase, and wherein the at least one gene is rendered less functional or knocked out as described herein. In another preferred embodiment of the method and/or cell of the disclosure, at least one gene involved in the synthesis and/or import of a co-factor that is involved in synthesis of LBA, a modified form of LBA, a glycosylated form of LBA, fucosylated LBA and/or sialylated LBA is rendered less functional or knocked out, preferably the co-factor is selected from the list comprising, consisting of or consisting essentially of pyrroloquinoline quinone (PQQ), FAD, a divalent metal cation, Ca²⁺, Cu²⁺, Mg²⁺, Zn²⁺, heme, heme C and pyridoxal 5′-phosphate. In a more preferred embodiment, at least one of the genes involved in synthesis of LBA, a modified form of LBA, a glycosylated form of LBA, fucosylated LBA and/or sialylated LBA and/or involved in the synthesis and/or import of a co-factor that is involved in synthesis of LBA, a modified form of LBA, a glycosylated form of LBA, fucosylated LBA and/or sialylated LBA is rendered less functional by insertion, deletion and/or modification of one or more nucleotide(s) in one or more polynucleotide sequence(s) selected from the list comprising, consisting of or consisting essentially of promoter sequence, ribosome binding site, untranslated region, coding sequence and transcription terminator sequence of the at least one gene. It is to be understood herein that by rendering at least one of the genes less functional or by knocking out at least one of the genes in the cell renders the cell with a less functional or knocked out synthesis of LBA, a modified form of LBA, a glycosylated form of LBA, fucosylated LBA and/or sialylated LBA. As a result, the cell produces less LBA, a modified form of LBA, a glycosylated form of LBA, fucosylated LBA and/or sialylated LBA compared to a cell wherein no one of the genes is rendered less functional of is knocked out. The term “less LBA” can be understood to comprise 0 g/L LBA. The term “less of a modified form of LBA” can be understood to comprise 0 g/L of the modified form of LBA. The term “less of a glycosylated form of LBA” can be understood to comprise 0 g/L of the glycosylated form of LBA. The term “less of fucosylated LBA” can be understood to comprise 0 g/L of fucosylated LBA. The term “less of sialylated LBA” can be understood to comprise 0 g/L of sialylated LBA.

In a more preferred embodiment, synthesis of LBA, a modified form of LBA, a glycosylated form of LBA, fucosylated LBA and/or sialylated LBA is obtained by expression of at least two genes selected from the list consisting of genes encoding carbohydrate oxidase, dehydrogenase, lactonase, oxygen-dependent FAD-linked oxidoreductase, and pyrroloquinoline quinone (PQQ) oxidoreductase, and wherein i) at least one of the at least two genes is rendered less functional, ii) at least two of the at least two genes is rendered less functional, iii) all of the at least two genes are rendered less functional, iv) at least one of the at least two genes is knocked out, ii) at least two of the at least two genes is knocked out, iii) all of the at least two genes are knocked out as described herein.

In another more preferred embodiment, the dehydrogenase is selected from the list comprising, consisting of or consisting essentially of lactose dehydrogenase, quinoprotein glucose dehydrogenase, aldose sugar dehydrogenase, glucose/fructose dehydrogenase, glucose/sorbosone dehydrogenase, cellobiose dehydrogenase, pyrroloquinoline quinone (PQQ) dehydrogenase, and malate dehydrogenase. In another and/or additional more preferred embodiment, the carbohydrate oxidase is a lactose oxidase.

In another and/or additional more preferred embodiment of the method and/or cell of the disclosure, the at least one gene encodes an enzyme wherein the enzyme is selected from an enzyme class selected from the list comprising, consisting of or consisting essentially of EC:1.1.3.-, EC:1.1.3.5, EC:1.1.3.4, EC:1.1.5.-, EC:1.1.5.2, and EC:1.1.99.18.

In another and/or additional more preferred embodiment of the method and/or cell of the disclosure, the at least one gene encodes an enzyme wherein the enzyme comprises a polypeptide sequence comprising an IPR domain selected from the list comprising, consisting of or consisting essentially of IPR000172, IPR001479, IPR002372, IPR006094, IPR007867, IPRO11041, IPRO11042, IPRO11047, IPR012132, IPR012938, IPR012951, 1PR013154, IPR013428, IPR015402, IPR015920, IPR016166, IPR016169, IPR017511, IPR017512, IPR018391, IPR027424, IPR029056, IPRO31640, IPR036188 and IPR036318 as defined by InterPro 90.0 as released on 4 Aug. 2022.

In another and/or additional more preferred embodiment of the method and/or cell of the disclosure, the at least one gene encodes an enzyme wherein the enzyme comprises a polypeptide sequence comprising a Panther domain selected from the list comprising, consisting of or consisting essentially of PTHR11552, PTHR13460, PTHR32303:SF4, PTHR42973 and PTHR47190 as defined by InterPro 90.0 as released on 4th August 2022.

In another and/or additional more preferred embodiment of the method and/or cell of the disclosure, the at least one gene encodes an enzyme wherein the enzyme comprises a polypeptide sequence comprising a PFAM domain selected from the list comprising, consisting of or consisting essentially of PF00732, PF01011, PF05199, PF07995, PF08031, PF08240, PF13360, PF13570, PF01565, PF16010 and PF16912 as defined by InterPro 90.0 as released on 4^{th} August 2022.

In another and/or additional more preferred embodiment of the method and/or cell of the disclosure, the at least one gene encodes an enzyme wherein the enzyme comprises a polypeptide sequence comprising the conserved protein domain cd10280 as defined by InterPro 90.0 as released on 4 Aug. 2022.

In another and/or additional more preferred embodiment of the method and/or cell of the disclosure, the at least one gene encodes an enzyme wherein the enzyme on 4 Aug. 2022, is part of a NOG family selected from the list comprising, consisting of or consisting essentially of COG0277, COG2133, COG2303 and COG4993 as defined by eggNOG5.0 as released in 2019.

In another and/or additional more preferred embodiment of the method and/or cell of the disclosure, the at least one gene encodes an enzyme wherein the enzyme uses a cofactor selected from the list comprising, consisting of or consisting essentially of pyrroloquinoline quinone (PQQ), FAD, a divalent metal cation, Ca²⁺, Cu²⁺, Mg²⁺, Zn²⁺, heme, heme C and pyridoxal 5′-phosphate.

In an even more preferred embodiment, the at least one gene encodes an enzyme wherein the enzyme is selected from the enzyme class EC:1.1.5.2, comprises a polypeptide sequence comprising, consisting of or consisting essentially of the IPR domains IPR001479, IPR002372, IPR011047, IPR017511 and IPR018391, as defined by InterPro 90.0 as released on 4 Aug. 2022, comprises a polypeptide sequence comprising, consisting of or consisting essentially of the Panther domain PTHR32303:SF4 as defined by InterPro 90.0 as released on 4 Aug. 2022, comprises a polypeptide sequence comprising, consisting of or consisting essentially of the PFAM domain PF01011 as defined by InterPro 90.0 as released on 4 Aug. 2022, comprises a polypeptide sequence comprising, consisting of or consisting essentially of the conserved protein domain cd10280 as defined by InterPro 90.0 as released on 4 Aug. 2022, and is part of the NOG family COG4993 as defined by eggNOG5.0 as released in 2019.

In an even more preferred embodiment, the at least one gene encodes an enzyme wherein the enzyme is selected from the enzyme class EC:1.1.5.2, comprises a polypeptide sequence comprising, consisting of or consisting essentially of the IPR domains IPR001479, IPR002372, IPR011047, IPR017511, IPR017512 and IPR018391, as defined by InterPro 90.0 as released on 4 Aug. 2022, comprises a polypeptide sequence comprising, consisting of or consisting essentially of the Panther domain PTHR32303:SF4 as defined by InterPro 90.0 as released on 4 Aug. 2022, comprises a polypeptide sequence comprising, consisting of or consisting essentially of the PFAM domains PF01011, PF13360 and PF13570 as defined by InterPro 90.0 as released on 4 Aug. 2022, comprises a polypeptide sequence comprising, consisting of or consisting essentially of the conserved protein domain cd10280 as defined by InterPro 90.0 as released on 4 Aug. 2022, and is part of the NOG family COG4993 as defined by eggNOG5.0 as released in 2019.

In another even more preferred embodiment, the at least one gene encodes an enzyme wherein the enzyme is selected from the enzyme class EC:1.1.5.-, comprises a polypeptide sequence comprising, consisting of or consisting essentially of the IPR domains IPR011041, IPR011042 and IPR012938, as defined by InterPro 90.0 as released on 4 Aug. 2022, comprises a polypeptide sequence comprising, consisting of or consisting essentially of the PFAM domain PF07995 as defined by InterPro 90.0 as released on 4 Aug. 2022 and is part of the NOG family COG2133, as defined by eggNOG5.0 as released in 2019.

In another even more preferred embodiment, the at least one gene encodes an enzyme wherein the enzyme is selected from the enzyme class EC:1.1.5.2, comprises a polypeptide sequence comprising, consisting of or consisting essentially of the IPR domains IPR011041, IPR011042 and IPR012938, as defined by InterPro 90.0 as released on 4 Aug. 2022 and comprises a polypeptide sequence comprising, consisting of or consisting essentially of the PFAM domain PF07995 as defined by InterPro 90.0 as released on 4 Aug. 2022.

In another even more preferred embodiment, the at least one gene encodes an enzyme that is a dehydrogenase and that uses a cofactor selected from the list comprising, consisting of or consisting essentially of pyrroloquinoline quinone (PQQ), FAD, a divalent metal cation, Ca²⁺, Cu²⁺, Mg²⁺, Zn²⁺, heme, heme C and pyridoxal 5′-phosphate. In another even more preferred embodiment, the at least one gene encodes an enzyme that i) is a quinoprotein glucose dehydrogenase or an aldose sugar dehydrogenase and ii) uses a cofactor selected from the list comprising, consisting of or consisting essentially of pyrroloquinoline quinone (PQQ), FAD, a divalent metal cation, Ca²⁺, Cu²⁺, Mg²⁺, Zn²⁺, heme, heme C and pyridoxal 5′-phosphate. In a most preferred embodiment, the at least one gene encodes a dehydrogenase like, e.g., the gcd gene from E. coli, the gdhB 2 gene from Streptomyces fradiae (UniProt ID A0A1Y2NTL5) or the BA894_20530 gene from Vibrio natriegens (UniProt ID A0A1B1EJ52).

In another even more preferred embodiment, the at least one gene encodes an enzyme wherein the enzyme comprises a polypeptide sequence comprising an IPR domain selected from the list comprising, consisting of or consisting essentially of IPR000172, IPR001479, IPR007867, IPR011041, IPR011047, IPR013154, IPR013428, IPR015402, IPR017511, IPR029056 and IPR031640 as defined by InterPro 90.0 as released on 4 Aug. 2022.

In another even more preferred embodiment, the at least one gene encodes an enzyme wherein the enzyme comprises a polypeptide sequence comprising a PFAM domain selected from the list comprising, consisting of or consisting essentially of PF00732, PF05199, PF08240 and PF16912 as defined by InterPro 90.0 as released on 4 Aug. 2022.

In another even more preferred embodiment, the at least one gene encodes an enzyme wherein the enzyme comprises the Panther domain PTHR13460 as defined by InterPro 90.0 as released on 4 Aug. 2022.

In another even more preferred embodiment, the at least one gene encodes an enzyme wherein the enzyme is selected from an enzyme class selected from the list comprising, consisting of or consisting essentially of EC:1.1.3.- and EC:1.1.3.5, EC:1.1.3.4, comprises a polypeptide sequence comprising an IPR domain selected from the list comprising, consisting of or consisting essentially of IPR000172, IPR006094, IPR007867, IPR012132, IPR012951, IPR016166, IPR016169, IPR027424, IPR036188 and IPR036318 as defined by InterPro 90.0 as released on 4 Aug. 2022, comprises a polypeptide sequence comprising a PFAM domain selected from the list comprising, consisting of or consisting essentially of PF00732, PF05199, PF08031 and PF01565 as defined by InterPro 90.0 as released on 4 Aug. 2022, and uses FAD as cofactor.

In another even more preferred embodiment, the at least one gene encodes an enzyme wherein the enzyme is selected from the enzyme class EC:1.1.99.18, comprises a polypeptide sequence comprising an IPR domain selected from the list comprising, consisting of or consisting essentially of, IPR000172, IPR007867, IPR015920 and IPR036188 as defined by InterPro 90.0 as released on 4 Aug. 2022, and comprises a polypeptide sequence comprising a PFAM domain selected from the list comprising, consisting of or consisting essentially of PF00732, PF05199 and PF16010 as defined by InterPro 90.0 as released on 4 Aug. 2022.

In an additional specific embodiment, the cell is genetically engineered for production of a saccharide, wherein the cell comprises a pathway for production of the saccharide. In a preferred embodiment, the cell is genetically engineered for production of two or more saccharides. In another and/or additional preferred embodiment, the cell is genetically engineered for an enhanced production of a saccharide, an enhanced uptake of one or more precursor(s) and/or acceptor(s) that is/are used in the synthesis of a saccharide, a better efflux of the saccharide, a decreased production of by-products like, e.g., acids, an increased availability of co-factors like, e.g., ATP, NADP, NADPH, and/or better metabolic flux through any one of the sialylation, fucosylation, galactosylation, N-acetylglucosaminylation, N-acetylgalactosaminylation, mannosylation, and/or N-acetylmannosaminylation pathway present in the cell.

In a preferred aspect of the method and/or cell of the disclosure, the genetically engineered cell is modified with gene expression modules wherein the expression from any one of the expression modules is constitutive or is tunable.

The expression modules are also known as transcriptional units and comprise polynucleotides for expression of recombinant genes including coding gene sequences and appropriate transcriptional and/or translational control signals that are operably linked to the coding genes. The control signals comprise promoter sequences, untranslated regions, ribosome binding sites, terminator sequences. The expression modules can contain elements for expression of one single recombinant gene but can also contain elements for expression of more recombinant genes or can be organized in an operon structure for integrated expression of two or more recombinant genes. The polynucleotides may be produced by recombinant DNA technology using techniques well-known in the art. Methods that are well known to those skilled in the art to construct expression modules include, for example, in vitro recombinant DNA techniques, synthetic techniques, and in vivo genetic recombination. See, for example, the techniques described in Sambrook et al. (2001) Molecular Cloning: a laboratory manual, 3rd Edition, Cold Spring Harbor Laboratory Press, CSH, New York or to Current Protocols in Molecular Biology, John Wiley and Sons, N.Y. (1989 and yearly updates).

According to a preferred aspect of the disclosure, the cell is modified with one or more expression modules. The expression modules can be integrated in the genome of the cell or can be presented to the cell on a vector. The vector can be present in the form of a plasmid, cosmid, phage, liposome, or virus, which is to be stably transformed/transfected into the metabolically engineered cell. Such vectors include, among others, chromosomal, episomal and virus-derived vectors, e.g., vectors derived from bacterial plasmids, from bacteriophage, from transposons, from yeast episomes, from insertion elements, from yeast chromosomal elements, from viruses, and vectors derived from combinations thereof, such as those derived from plasmid and bacteriophage genetic elements, such as cosmids and phagemids. These vectors may contain selection markers such as but not limited to antibiotic markers, auxotrophic markers, toxin-antitoxin markers, RNA sense/antisense markers. The expression system constructs may contain control regions that regulate as well as engender expression. Generally, any system or vector suitable to maintain, propagate or express polynucleotides and/or to express a polypeptide in a host may be used for expression in this regard. The appropriate DNA sequence may be inserted into the expression system by any of a variety of well-known and routine techniques, such as, for example, those set forth in Sambrook et al., see above. For recombinant production, cells can be genetically engineered to incorporate expression systems or portions thereof or polynucleotides of the disclosure. Introduction of a polynucleotide into the cell can be effected by methods described in many standard laboratory manuals, such as, e.g., Sambrook et al., 1989, supra.

As used herein an expression module comprises polynucleotides for expression of at least one recombinant gene. The recombinant gene is involved in the pathway for production of a saccharide; or the recombinant gene is linked to other pathways in the cell that are not involved in the synthesis of a saccharide. The recombinant genes encode endogenous proteins with a modified expression or activity, preferably the endogenous proteins are overexpressed; or the recombinant genes encode heterologous proteins that are heterogeneously introduced and expressed in the modified cell, preferably overexpressed. The endogenous proteins can have a modified expression in the cell that also expresses a heterologous protein.

According to a preferred aspect of the disclosure, the expression of each of the expression modules is constitutive or tuneable as defined herein.

In a preferred embodiment of the method and/or cell of the disclosure, the pathway for production of the saccharide is selected from the list comprising, consisting of or consisting essentially of fucosylation pathway, sialylation pathway, galactosylation pathway, N-acetylglucosaminylation pathway, N-acetylgalactosaminylation pathway, mannosylation pathway and N-acetylmannosaminylation pathway. In a more preferred embodiment, the cell is genetically engineered to comprise at least one of the pathway(s). In an even more preferred embodiment, the cell comprises at least one of the pathway(s) wherein at least one of the pathway(s) has/have been genetically engineered.

In another and/or additional preferred embodiment of the method and/or cell, the cell comprises a fucosylation pathway. In a more preferred additional and/or alternative embodiment, the cell is metabolically engineered to comprise a fucosylation pathway. In another even more preferred additional and/or alternative embodiment, the cell has been metabolically engineered to comprise a fucosylation pathway wherein any one or more of the genes is/are selected from the list comprising, consisting of or consisting essentially of mannose-6-phosphate isomerase, phosphomannomutase, mannose-1-phosphate guanylyltransferase, GDP-mannose 4,6-dehydratase, GDP-L-fucose synthase, fucose permease, fucose kinase, fucose-1-phosphate guanylyltransferase and fucosyltransferase has/have a modified and/or enhanced expression.

In another and/or additional preferred embodiment of the method and/or cell, the cell comprises a sialyation pathway. In an even more preferred embodiment, the cell is metabolically engineered to comprise a sialylation pathway. In another even more preferred embodiment, the cell has been metabolically engineered to comprise a sialylation pathway wherein any one or more of the genes is/are selected from the list comprising, consisting of or consisting essentially of L-glutamine-D-fructose-6-phosphate aminotransferase, phosphoglucosamine mutase, N-acetylglucosamine-6-P deacetylase, N-acylglucosamine 2-epimerase, UDP-N-acetylglucosamine 2-epimerase, N-acetylmannosamine-6-phosphate 2-epimerase, UDP-GlcNAc 2-epimerase/kinase, glucosamine 6-phosphate N-acetyltransferase, N-acetylglucosamine-6-phosphate phosphatase, phosphoacetylglucosamine mutase, N-acetylglucosamine 1-phosphate uridylyltransferase, glucosamine-1-phosphate acetyltransferase, Neu5Ac synthase, N-acetylneuraminate lyase, N-acylneuraminate-9-phosphate synthase, N-acylneuraminate-9-phosphatase, sialic acid transporter, CMP kinase, CMP-sialic acid synthase and sialyltransferase has/have a modified and/or enhanced expression.

In another and/or additional preferred embodiment of the method and/or cell, the cell comprises a galactosylation pathway. In a more preferred additional and/or alternative embodiment, the cell is metabolically engineered to comprise a galactosylation pathway. In another even more preferred additional and/or alternative embodiment, the cell has been metabolically engineered to comprise a galactosylation pathway wherein any one or more of the genes is/are selected from the list comprising, consisting of or consisting essentially of galactose-1-epimerase, galactokinase, glucokinase, galactose-1-phosphate uridylyltransferase, UDP-glucose 4-epimerase, glucose-1-phosphate uridylyltransferase, phosphoglucomutase and galactosyltransferase has/have a modified and/or enhanced expression.

In another and/or additional preferred embodiment of the method and/or cell, the cell comprises an “N-acetylglucosaminylation” pathway. In a more preferred additional and/or alternative embodiment, the cell is metabolically engineered to comprise an N-acetylglucosaminylation pathway. In another even more preferred additional and/or alternative embodiment, the cell has been metabolically engineered to comprise an N-acetylglucosaminylation pathway wherein any one or more of the genes is/are selected from the list comprising, consisting of or consisting essentially of L-glutamine-D-fructose-6-phosphate aminotransferase, N-acetylglucosamine-6-phosphate deacetylase, phosphoglucosamine mutase, N-acetylglucosamine-1-phosphate uridylyltransferase, glucosamine-1-phosphate acetyltransferase and a glycosyltransferase transferring GlcNAc has/have a modified and/or enhanced expression.

In another and/or additional preferred embodiment of the method and/or cell, the cell comprises an “N-acetylgalactosaminylation” pathway. In a more preferred additional and/or alternative embodiment, the cell is metabolically engineered to comprise an N-acetylgalactosaminylation pathway. In another even more preferred additional and/or alternative embodiment, the cell has been metabolically engineered to comprise an N-acetylgalactosaminylation pathway wherein any one or more of the genes is/are selected from the list comprising, consisting of or consisting essentially of L-glutamine-D-fructose-6-phosphate aminotransferase, phosphoglucosamine mutase, N-acetylglucosamine 1-phosphate uridylyltransferase, glucosamine-1-phosphate acetyltransferase, UDP-N-acetylglucosamine 4-epimerase, UDP-glucose 4-epimerase, N-acetylgalactosamine kinase and/or UDP-N-acetylgalactosamine pyrophosphorylase and a glycosyltransferase transferring GalNAc has/have a modified and/or enhanced expression.

In another and/or additional preferred embodiment of the method and/or cell, the cell comprises a “mannosylation” pathway. In a more preferred additional and/or alternative embodiment, the cell is metabolically engineered to comprise a mannosylation pathway. In another even more preferred additional and/or alternative embodiment, the cell has been metabolically engineered to comprise a mannosylation pathway wherein any one or more of the genes is/are selected from the list comprising, consisting of or consisting essentially of mannose-6-phosphate isomerase, phosphomannomutase and/or mannose-1-phosphate guanylyltransferase and mannosyltransferase has/have a modified and/or enhanced expression.

In another and/or additional preferred embodiment of the method and/or cell, the cell comprises an “N-acetylmannosaminylation” pathway. In a more preferred additional and/or alternative embodiment, the cell is metabolically engineered to comprise an N-acetylmannosaminylation pathway. In another even more preferred additional and/or alternative embodiment, the cell has been metabolically engineered to comprise an N-acetylmannosaminylation pathway wherein any one or more of the genes is/are selected from the list comprising, consisting of or consisting essentially of L-glutamine-D-fructose-6-phosphate aminotransferase, glucosamine-6-phosphate deaminase, phosphoglucosamine mutase, N-acetylglucosamine-6-phosphate deacetylase, glucosamine 6-phosphate N-acetyltransferase, N-acetylglucosamine-1-phosphate uridyltransferase, glucosamine-1-phosphate acetyltransferase, glucosamine-1-phosphate acetyltransferase, UDP-GlcNAc 2-epimerase and/or ManNAc kinase and a glycosyltransferase transferring ManNAc has/have a modified and/or enhanced expression.

In another and/or additional preferred embodiment of the method and/or cell of the disclosure, the cell comprises one or more pathway(s) for monosaccharide synthesis. The pathways for monosaccharide synthesis comprise, consist of or consist essentially of enzymes like, e.g., carboxylases, decarboxylases, isomerases, epimerases, reductases, enolases, phosphorylases, carboxykinases, kinases, phosphatases, aldolases, hydrolases, dehydrogenases, enzymes involved in the synthesis of one or more nucleoside triphosphate(s) like UTP, GTP, ATP and CTP, enzymes involved in the synthesis of any one or more nucleoside mono- or diphosphates like, e.g., UMP and UDP, respectively, and enzymes involved in the synthesis of phosphoenolpyruvate (PEP).

In another and/or additional preferred embodiment of the method and/or cell of the disclosure, the cell comprises one or more pathway(s) for phosphorylated monosaccharide synthesis. The pathways for phosphorylated monosaccharide synthesis comprise, consist of or consist essentially of enzymes involved in the synthesis of one or more monosaccharide(s), one or more nucleoside mono-, di- and/or triphosphate(s) and enzymes involved in the synthesis of phosphoenolpyruvate (PEP) like, e.g., but not limited to PEP synthase, carboxylases, decarboxylases, isomerases, epimerases, reductases, enolases, phosphorylases, carboxykinases, kinases, phosphatases, aldolases, hydrolases and dehydrogenases. In another and/or additional preferred embodiment of the method and/or cell of the disclosure, the cell comprises one or more pathways for the synthesis of one or more nucleotide-activated sugars. The pathways for nucleotide-activated sugar synthesis comprise enzymes like, e.g., PEP synthase, carboxylases, decarboxylases, isomerases, epimerases, reductases, enolases, phosphorylases, carboxykinases, kinases, phosphatases, aldolases, hydrolases, dehydrogenases, mannose-6-phosphate isomerase, phosphomannomutase, mannose-1-phosphate guanylyltransferase, GDP-mannose 4,6-dehydratase, GDP-L-fucose synthase, L-fucokinase/GDP-fucose pyrophosphorylase, L-glutamine-D-fructose-6-phosphate aminotransferase, glucosamine-6-phosphate deaminase, phosphoglucosamine mutase, N-acetylglucosamine-6-phosphate deacetylase, N-acetylglucosamine epimerase, UDP-N-acetylglucosamine 2-epimerase, N-acetylglucosamine-6P 2-epimerase, glucosamine 6-phosphate N-acetyltransferase, N-acetylglucosamine-6-phosphate phosphatase, N-acetylmannosamine-6-phosphate phosphatase, N-acetylmannosamine kinase, phosphoacetylglucosamine mutase, N-acetylglucosamine-1-phosphate uridyltransferase, glucosamine-1-phosphate acetyltransferase, sialic acid synthase, N-acetylneuraminate lyase, N-acylneuraminate-9-phosphate synthase, N-acylneuraminate-9-phosphate phosphatase, CMP-sialic acid synthase, galactose-1-epimerase, galactokinase, glucokinase, galactose-1-phosphate uridylyltransferase, UDP-glucose 4-epimerase, glucose-1-phosphate uridylyltransferase, glucophosphomutase and/orN-acetylglucosamine-1-phosphate uridylyltransferase.

In another and/or additional preferred embodiment of the method and/or cell, the cell possesses, preferably expresses, more preferably overexpresses, one or more glycosyltransferase(s) selected from the list comprising, consisting of or consisting essentially of fucosyltransferases, sialyltransferases, galactosyltransferases, glucosyltransferases, mannosyltransferases, N-acetylglucosaminyltransferases, N-acetylgalactosaminyltransferases, N-acetylmannosaminyltransferases, xylosyltransferases, glucuronyltransferases, galacturonyltransferases, glucosaminyltransferases, N-glycolylneuraminyltransferases, rhamnosyltransferases, N-acetylrhamnosyltransferases, UDP-4-amino-4,6-dideoxy-N-acetyl-β-L-altrosamine transaminases, UDP-N-acetylglucosamine enolpyruvyl transferases and fucosaminyltransferases.

In a more preferred embodiment of the method and/or cell of the disclosure, the fucosyltransferase is selected from the list comprising, consisting of or consisting essentially of α-1,2-fucosyltransferase, α-1,3-fucosyltransferase, α-1,4-fucosyltransferase and α-1,6-fucosyltransferase.

In an alternative and/or additional more preferred embodiment of the method and/or cell of the disclosure, the sialyltransferase is selected from the list comprising, consisting of or consisting essentially of α-2,3-sialyltransferase, α-2,6-sialyltransferase, and α-2,8-sialyltransferase.

In an alternative and/or additional more preferred embodiment of the method and/or cell of the disclosure, the galactosyltransferase is selected from the list comprising, consisting of or consisting essentially of β-1,3-galactosyltransferase, N-acetylglucosamine β-1,3-galactosyltransferase, β-1,4-galactosyltransferase, N-acetylglucosamine-1,4-galactosyltransferase, α-1,3-galactosyltransferase and β-1,4-galactosyltransferase.

In an alternative and/or additional more preferred embodiment of the method and/or cell of the disclosure, the glucosyltransferase is selected from the list comprising, consisting of or consisting essentially of α-glucosyltransferase, β-1,2-glucosyltransferase, β-1,3-glucosyltransferase and β-1,4-glucosyltransferase.

In an alternative and/or additional more preferred embodiment of the method and/or cell of the disclosure, the mannosyltransferase is selected from the list comprising, consisting of or consisting essentially of α-1,2-mannosyltransferase, α-1,3-mannosyltransferase and α-1,6-mannosyltransferase.

In an alternative and/or additional more preferred embodiment of the method and/or cell of the disclosure, the N-acetylglucosaminyltransferase is selected from the list comprising, consisting of or consisting essentially of galactoside β-1,3-N-acetylglucosaminyltransferase and β-1,6-N-acetylglucosaminyltransferase.

In an alternative and/or additional more preferred embodiment of the method and/or cell of the disclosure, the N-acetylgalactosaminyltransferase is selected from the list comprising, consisting of or consisting essentially of α-1,3-N-acetylgalactosaminyltransferase.

In an alternative and/or additional more preferred embodiment of the method and/or cell of the disclosure, the cell is modified in the expression or activity of at least one of the glycosyltransferases. In a preferred embodiment, the glycosyltransferase is an endogenous protein of the cell with a modified expression or activity, preferably the endogenous glycosyltransferase is overexpressed; alternatively the glycosyltransferase is a heterologous protein that is heterogeneously introduced and expressed in the cell, preferably overexpressed. The endogenous glycosyltransferase can have a modified expression in the cell that also expresses a heterologous glycosyltransferase.

In another and/or additional preferred embodiment of the method and/or cell, the cell is capable of producing, preferably produces, one or more nucleotide-activated sugars, preferably the cell is genetically engineered for production of one or more of the nucleotide-activated sugar(s). Herein, the one or more nucleotide-activated sugar(s) is/are selected from the list comprising, consisting of or consisting essentially of UDP-N-acetylglucosamine (UDP-GlcNAc), UDP-N-acetylgalactosamine (UDP-GalNAc), UDP-N-acetylmannosamine (UDP-ManNAc), UDP-glucose (UDP-Glc), UDP-galactose (UDP-Gal), GDP-mannose (GDP-Man), GDP-fucose, (GDP-Fuc), UDP-glucuronate, UDP-galacturonate, UDP-2-acetamido-2,6-dideoxy-L-arabino-4-hexulose, UDP-2-acetamido-2,6-dideoxy-L-lyxo-4-hexulose, UDP-N-acetyl-L-rhamnosamine (UDP-L-RhaNAc or UDP-2-acetamido-2,6-dideoxy-L-mannose), dTDP-N-acetylfucosamine, UDP-N-acetylfucosamine (UDP-L-FucNAc or UDP-2-acetamido-2,6-dideoxy-L-galactose), UDP-N-acetyl-L-pneumosamine (UDP-L-PneNAC or UDP-2-acetamido-2,6-dideoxy-L-talose), UDP-N-acetylmuramic acid, UDP-N-acetyl-L-quinovosamine (UDP-L-QuiNAc or UDP-2-acetamido-2,6-dideoxy-L-glucose), CMP-sialic acid (CMP-Neu5Ac), CMP-Neu4Ac, CMP-Neu5Ac9N₃, CMP-Neu4,5Ac2, CMP-Neu5,7Ac2, CMP-Neu5,9Ac2, CMP-Neu5,7(8,9)Ac₂, CMP-N-glycolylneuraminic acid (CMP-Neu5Gc), GDP-rhamnose and UDP-xylose.

In another and/or additional preferred embodiment of the method and/or cell, the cell comprises a pathway for the synthesis of a nucleotide-activated sugar selected from the list comprising, consisting of or consisting essentially of UDP-N-acetylglucosamine (UDP-GlcNAc), UDP-N-acetylgalactosamine (UDP-GalNAc), UDP-N-acetylmannosamine (UDP-ManNAc), UDP-glucose (UDP-Glc), UDP-galactose (UDP-Gal), GDP-mannose (GDP-Man), GDP-fucose, (GDP-Fuc), UDP-glucuronate, UDP-galacturonate, UDP-2-acetamido-2,6-dideoxy-L-arabino-4-hexulose, UDP-2-acetamido-2,6-dideoxy-L-lyxo-4-hexulose, UDP-N-acetyl-L-rhamnosamine (UDP-L-RhaNAc or UDP-2-acetamido-2,6-dideoxy-L-mannose), dTDP-N-acetylfucosamine, UDP-N-acetylfucosamine (UDP-L-FucNAc or UDP-2-acetamido-2,6-dideoxy-L-galactose), UDP-N-acetyl-L-pneumosamine (UDP-L-PneNAC or UDP-2-acetamido-2,6-dideoxy-L-talose), UDP-N-acetylmuramic acid, UDP-N-acetyl-L-quinovosamine (UDP-L-QuiNAc or UDP-2-acetamido-2,6-dideoxy-L-glucose), CMP-sialic acid (CMP-Neu5Ac), CMP-Neu4Ac, CMP-Neu5Ac9N₃, CMP-Neu4,5Ac₂, CMP-Neu5,7Ac₂, CMP-Neu5,9Ac₂, CMP-Neu5,7(8,9)Ac₂, CMP-N-glycolylneuraminic acid (CMP-Neu5Gc), GDP-rhamnose and UDP-xylose.

The cell used herein is optionally genetically modified to express the de novo synthesis of UDP-GlcNAc. UDP-GlcNAc can be provided by an enzyme expressed in the cell or by the metabolism of the cell. Such cell producing an UDP-GlcNAc can express enzymes converting, e.g., GlcNAc, which is to be added to the cell, to UDP-GlcNAc. These enzymes may be any one or more of the list comprising, consisting of or consisting essentially of an N-acetyl-D-glucosamine kinase, an N-acetylglucosamine-6-phosphate deacetylase, a phosphoglucosamine mutase, and an N-acetylglucosamine-1-phosphate uridyltransferase/glucosamine-1-phosphate acetyltransferase from several species including Homo sapiens, Escherichia coli. Preferably, the cell is modified to produce UDP-GlcNAc. More preferably, the cell is modified for enhanced UDP-GlcNAc production. The modification can be any one or more selected from the list comprising, consisting of or consisting essentially of knock-out of an N-acetylglucosamine-6-phosphate deacetylase, over-expression of an L-glutamine-D-fructose-6-phosphate aminotransferase, over-expression of a phosphoglucosamine mutase, and over-expression of an N-acetylglucosamine-1-phosphate uridyltransferase/glucosamine-1-phosphate acetyltransferase.

Additionally, or alternatively, the cell used herein is optionally genetically modified to express the de novo synthesis of CMP-Neu5Ac. CMP-Neu5Ac can be provided by an enzyme expressed in the cell or by the metabolism of the cell. Such cell producing CMP-Neu5Ac can express an enzyme converting, e.g., sialic acid to CMP-Neu5Ac. This enzyme may be a CMP-sialic acid synthetase, like the N-acylneuraminate cytidylyltransferase from several species including Homo sapiens, Neisseria meningitidis, and Pasteurella multocida. Preferably, the cell is modified to produce CMP-Neu5Ac. More preferably, the cell is modified for enhanced CMP-Neu5Ac production. The modification can be any one or more selected from the list comprising, consisting of or consisting essentially of knock-out of an N-acetylglucosamine-6-phosphate deacetylase, knock-out of a glucosamine-6-phosphate deaminase, over-expression of a CMP-sialic acid synthetase, and over-expression of an N-acetyl-D-glucosamine-2-epimerase encoding gene.

Additionally, or alternatively, the cell used herein is optionally genetically modified to express the de novo synthesis of GDP-fucose. GDP-fucose can be provided by an enzyme expressed in the cell or by the metabolism of the cell. Such cell producing GDP-fucose can express an enzyme converting, e.g., fucose, which is to be added to the cell, to GDP-fucose. This enzyme may be, e.g., a bifunctional fucose kinase/fucose-1-phosphate guanylyltransferase, like Fkp from Bacteroides fragilis, or the combination of one separate fucose kinase together with one separate fucose-1-phosphate guanylyltransferase like they are known from several species including Homo sapiens, Sus scrofa and Rattus norvegicus. Preferably, the cell is modified to produce GDP-fucose. More preferably, the cell is modified for enhanced GDP-fucose production. The modification can be any one or more selected from the list comprising, consisting of or consisting essentially of knock-out of an UDP-glucose:undecaprenyl-phosphate glucose-1-phosphate transferase encoding gene, over-expression of a GDP-L-fucose synthase encoding gene, over-expression of a GDP-mannose 4,6-dehydratase encoding gene, over-expression of a mannose-1-phosphate guanylyltransferase encoding gene, over-expression of a phosphomannomutase encoding gene and over-expression of a mannose-6-phosphate isomerase encoding gene.

Additionally, or alternatively, the cell used herein is optionally genetically modified to express the de novo synthesis of UDP-Gal. UDP-Gal can be provided by an enzyme expressed in the cell or by the metabolism of the cell. Such cell producing UDP-Gal can express an enzyme converting, e.g., UDP-glucose, to UDP-Gal. This enzyme may be, e.g., the UDP-glucose-4-epimerase GalE like as known from several species including Homo sapiens, Escherichia coli, and Rattus norvegicus. Preferably, the cell is modified to produce UDP-Gal. More preferably, the cell is modified for enhanced UDP-Gal production. The modification can be any one or more selected from the list comprising, consisting of or consisting essentially of knock-out of a bifunctional 5′-nucleotidase/UDP-sugar hydrolase encoding gene, knock-out of a galactose-1-phosphate uridylyltransferase encoding gene and over-expression of an UDP-glucose-4-epimerase encoding gene.

Additionally, or alternatively, the cell used herein is optionally genetically modified to express the de novo synthesis of UDP-GalNAc. UDP-GalNAc can be synthesized from UDP-GlcNAc by the action of a single-step reaction using an UDP-N-acetylglucosamine 4-epimerase like, e.g., wbgU from Plesiomonas shigelloides, gne from Yersinia enterocolitica or wbpP from Pseudomonas aeruginosa serotype 06. Preferably, the cell is modified to produce UDP-GalNAc. More preferably, the cell is modified for enhanced UDP-GalNAc production.

Additionally, or alternatively, the cell used herein is optionally genetically modified to express the de novo synthesis of UDP-ManNAc. UDP-ManNAc can be synthesized directly from UDP-GlcNAc via an epimerization reaction performed by an UDP-GlcNAc 2-epimerase (like, e.g., cap5P from Staphylococcus aureus, RffE from E. coli, Cps19fK from S. pneumoniae, and RfbC from S. enterica). Preferably, the cell is modified to produce UDP-ManNAc. More preferably, the cell is modified for enhanced UDP-ManNAc production.

In another and/or additional preferred embodiment of the method and/or cell, the cell possesses, preferably expresses, more preferably overexpresses, one or more genes selected from the list comprising, consisting of or consisting essentially of mannose-6-phosphate isomerase, phosphomannomutase, mannose-1-phosphate guanylyltransferase, GDP-mannose 4,6-dehydratase, GDP-L-fucose synthase, fucose permease, fucose kinase, fucose-1-phosphate guanylyltransferase, L-glutamine-D-fructose-6-phosphate aminotransferase, phosphoglucosamine mutase, N-acetylglucosamine-6-P deacetylase, N-acylglucosamine 2-epimerase, UDP-N-acetylglucosamine 2-epimerase, N-acetylmannosamine-6-phosphate 2-epimerase, UDP-GlcNAc 2-epimerase/kinase, glucosamine 6-phosphate N-acetyltransferase, N-acetylglucosamine-6-phosphate phosphatase, phosphoacetylglucosamine mutase, N-acetylglucosamine 1-phosphate uridylyltransferase, glucosamine-1-phosphate acetyltransferase, Neu5Ac synthase, N-acetylneuraminate lyase, N-acylneuraminate-9-phosphate synthase, N-acylneuraminate-9-phosphatase, sialic acid transporter, CMP kinase, CMP-sialic acid synthase, galactose-1-epimerase, galactokinase, glucokinase, galactose-1-phosphate uridylyltransferase, UDP-glucose 4-epimerase, glucose-1-phosphate uridylyltransferase, phosphoglucomutase, UDP-N-acetylglucosamine 4-epimerase, N-acetylgalactosamine kinase and UDP-N-acetylgalactosamine pyrophosphorylase.

In another and/or additional preferred embodiment of the method and/or cell, the cell is genetically engineered for production of a saccharide wherein the saccharide is selected from the list comprising, consisting of or consisting essentially of monosaccharide, phosphorylated monosaccharide, activated monosaccharide, disaccharide, oligosaccharide, neutral (non-charged) oligosaccharide, a negatively charged, preferably sialylated, oligosaccharide, milk oligosaccharide; sialylated milk oligosaccharide, neutral (non-charged) milk oligosaccharide, fucosylated milk oligosaccharide, non-fucosylated neutral (non-charged) milk oligosaccharide, sialylated mammalian milk oligosaccharide, neutral (non-charged) mammalian milk oligosaccharide, fucosylated mammalian milk oligosaccharide, non-fucosylated neutral (non-charged) mammalian milk oligosaccharide, sialylated human milk oligosaccharide, neutral (non-charged) human milk oligosaccharide, fucosylated human milk oligosaccharide, non-fucosylated neutral (non-charged) human milk oligosaccharide; O-antigen; enterobacterial common antigen (ECA); the oligosaccharide repeats present in capsular polysaccharides; peptidoglycan; an amino-sugar; Lewis-type antigen oligosaccharide; an antigen of the human ABO blood group system; an animal oligosaccharide; a plant oligosaccharide; fucosylated oligosaccharide; sialylated oligosaccharide; N-acetylglucosamine containing neutral (non-charged) saccharide; N-acetylglucosamine containing saccharide; N-acetyllactosamine containing saccharide; lacto-N-biose containing saccharide; non-fucosylated neutral (non-charged) saccharide; chitosan; chitosan comprising oligosaccharide; heparosan; chondroitin sulphate; glycosaminoglycan oligosaccharide; heparin; heparan sulphate; dermatan sulphate; hyaluronan; hyaluronic acid; and keratan sulphate.

In a more preferred embodiment, the saccharide is a mammalian milk oligosaccharide (MMO) as described herein. In another more preferred embodiment, the saccharide is a human milk oligosaccharide (HMO) as described herein. In another more preferred embodiment, the saccharide is an animal oligosaccharide selected from the list consisting of N-glycans and O-glycans. In another more preferred embodiment, the saccharide is a plant oligosaccharide selected from the list consisting of N-glycans and O-glycans. In the context of the disclosure, N-glycans and O-glycans refer to the oligosaccharide structures as known by the person skilled in the art wherein the structures are not attached to a protein or a peptide. In another more preferred embodiment, the fucosylated oligosaccharide is selected from the list comprising, consisting of or consisting essentially of 2′-fucosyllactose (2′FL), 3-fucosyllactose (3FL), 4-fucosyllactose (4FL), 6-fucosyllactose (6FL), 2′,3-difucosyllactose (diFL), lacto-N-fucopentaose I, lacto-N-neofucopentaose I, lacto-N-fucopentaose II, lacto-N-fucopentaose III, lacto-N-fucopentaose V, lacto-N-fucopentaose VI, lacto-N-neofucopentaose V, lacto-N-difucohexaose I, lacto-N-difucohexaose II, difucosyl-lacto-N-hexaose and difucosyl-lacto-N-neohexaose. In another more preferred embodiment, the sialylated oligosaccharide is selected from the list comprising, consisting of or consisting essentially of 3′sialyllactose (3′SL), 6′sialyllactose (6′SL), sialyllacto-N-tetraose a (LSTa), sialyllacto-N-tetraose b (LSTb), sialyllacto-N-tetraose c (LSTc), sialyllacto-N-tetraose d (LSTd), disialyllacto-N-tetraose, disialyllacto-N-neotetraose, monosialyllacto-N-hexaose, disialyllacto-N-hexaose I, disialyllacto-N-hexaose II, monosialyllacto-N-neohexaose I, monosialyllacto-N-neohexaose II, disialyllacto-N-neohexaose, 3′-sialyl-3-fucosyllactose, fucodisialyllacto-N-hexaose, disialomonofucosyllacto-N-neohexaose, sialyllacto-N-fucohexaose II, disialyllacto-N-fucopentaose II and monofucosyldisialyllacto-N-tetraose. In another more preferred embodiment, the N-acetylglucosamine containing neutral (non-charged) saccharide is selected from the list comprising, consisting of or consisting essentially of lacto-N-biose (LNB), N-acetyllactosamine (LacNAc), lacto-N-triose II (LN3), lacto-N-tetraose (LNT), lacto-N-neotetraose (LNnT), 6′-galactosyllactose, 3′-galactosyllactose, lacto-N-hexaose, lacto-N-neohexaose, para-lacto-N-hexaose, para-lacto-N-neohexaose.

In an even more preferred embodiment, the saccharide is selected from the list comprising, consisting of or consisting essentially of Fucα1-2Galβ1-3GlcNAc; Galβ1-3[Fucα1-4]GlcNAc; Fucα1-2Galβ1-3[Fucα1-4]GlcNAc; Neu5Acα2-3Galβ1-3[Fucα1-4]GlcNAc; Fucα1-2Galβ1-4GlcNAc; Galβ1-4[Fucα1-3]GlcNAc; Fucα1-2Galβ1-4[Fucα1-3]GlcNAc; Neu5Acα2-3Galβ1-4[Fucα1-3]GlcNAc; 2′-fucosyllactose (2′FL), 3-fucosyllactose (3FL), 4-fucosyllactose (4FL), 6-fucosyllactose (6FL), 2′,3-difucosyllactose (diFL), lacto-N-fucopentaose I, lacto-N-neofucopentaose I, lacto-N-fucopentaose II, lacto-N-fucopentaose III, lacto-N-fucopentaose V, lacto-N-fucopentaose VI, lacto-N-neofucopentaose V, lacto-N-difucohexaose I, lacto-N-difucohexaose II, difucosyl-lacto-N-hexaose and difucosyl-lacto-N-neohexaose, lacto-N-triose II (LN3), lacto-N-tetraose (LNT), lacto-N-neotetraose (LNnT), 6′-galactosyllactose, 3′-galactosyllactose, lacto-N-hexaose, lacto-N-neohexaose, para-lacto-N-hexaose, para-lacto-N-neohexaose, 3′sialyllactose (3′SL), 6′sialyllactose (6′SL), sialyllacto-N-tetraose a (LSTa), sialyllacto-N-tetraose b (LSTb), sialyllacto-N-tetraose c (LSTc), sialyllacto-N-tetraose d (LSTd), disialyllacto-N-tetraose, disialyllacto-N-neotetraose, monosialyllacto-N-hexaose, disialyllacto-N-hexaose I, disialyllacto-N-hexaose II, monosialyllacto-N-neohexaose I, monosialyllacto-N-neohexaose II, disialyllacto-N-neohexaose, 3′-sialyl-3-fucosyllactose, fucodisialyllacto-N-hexaose, disialomonofucosyllacto-N-neohexaose, sialyllacto-N-fucohexaose II, disialyllacto-N-fucopentaose II and monofucosyldisialyllacto-N-tetraose.

The saccharide in the context of the disclosure is preferably in free form, i.e., the saccharide does not contain any protective group.

In another and/or additional preferred embodiment of the method and/or cell of the disclosure, the cell is capable of producing, preferably produces, saccharide, LBA, modified form of LBA, glycosylated form of LBA, fucosylated LBA and/or sialylated LBA from one or more precursor(s) as defined herein. In a more preferred embodiment, the precursor is lactose. Preferably, one or more precursor(s) is/are fed to the cell from the culture or cultivation medium or the incubation. In another more preferred embodiment, the cell is capable of producing, preferably produces, at least one of the one or more precursor(s). In an even more preferred embodiment, the cell is capable of producing, preferably produces, all of the one or more precursor(s). In another more preferred embodiment, the cell is genetically engineered for the production of at least one of the one or more precursor(s). In an even more preferred embodiment, the cell is genetically engineered for the production of all of the one or more precursor(s). In another more preferred embodiment, at least one of the one or more precursor(s) is internalized in the cell via one or more membrane protein(s). In another preferred embodiment, the precursor(s) that is/are used by the cell for the production of the saccharide is/are completely converted into the saccharide.

In a preferred embodiment, the saccharide of the disclosure is produced by a cell that is cultured in a cell cultivation. Within the context of the disclosure, the cell cultivation comprises in vitro and/or ex vivo cultivation of cells. In another and/or additional more preferred embodiment, the cell cultivation is a fermentation. In an alternative and/or additional more preferred embodiment, the cell is cultivated or incubated in a reactor as defined herein. In an alternative and/or additional more preferred embodiment, the cell is cultivated or incubated in an incubator as defined herein.

In another and/or additional preferred embodiment, the cell is cultivated in culture or cultivation medium comprising, consisting of or consisting essentially of a carbon source comprising, consisting of or consisting essentially of a monosaccharide, disaccharide, oligosaccharide, polysaccharide, polyol, glycerol, a complex medium including molasses, corn steep liquor, peptone, tryptone or yeast extract. Preferably, the carbon source is selected from the list comprising, consisting of or consisting essentially of glucose, N-acetylglucosamine (GlcNAc), glycerol, fructose, sucrose, maltose, lactose, arabinose, malto-oligosaccharides, maltotriose, sorbitol, xylose, rhamnose, galactose, mannose, methanol, ethanol, trehalose, starch, cellulose, hemi-cellulose, molasses, corn-steep liquor, high-fructose syrup, acetate, citrate, lactate and pyruvate. In a more preferred embodiment, the culture or cultivation medium is a chemically defined medium. In an additional preferred embodiment, the culture or cultivation medium is a minimal salt medium comprising, consisting of or consisting essentially of sulphate, phosphate, chloride, ammonium, calcium, magnesium, sodium, potassium, iron, copper, zinc, manganese, cobalt, and/or selenium. In another and/or additional preferred embodiment, the cultivation or incubation medium comprises one or more precursor(s) that is/are used for production of the saccharide and/or the LBA. In a more preferred embodiment, the cultivation or incubation medium comprises one or more co-factor(s) selected from the list comprising, consisting of or consisting essentially of pyrroloquinoline quinone (PQQ), FAD, a divalent metal cation, Ca²⁺, Cu²⁺, Mg²⁺, Zn²⁺, heme, heme C and pyridoxal 5′-phosphate.

In another and/or additional preferred embodiment of the method of the disclosure, the method for production of a saccharide as described herein comprises at least one of the following steps:
i) Adding to the culture or cultivation medium in a reactor at least one precursor and/or acceptor feed wherein the total reactor volume ranges from 250 mL (milliliter) to 10,000 m³ (cubic meter), preferably in a continuous manner, and preferably so that the final volume of the culture or cultivation medium is not more than three-fold, preferably not more than two-fold, more preferably less than two-fold of the volume of the culture or cultivation medium before the addition of the precursor and/or acceptor feed;
ii) Adding at least one precursor and/or acceptor feed in a continuous manner to the culture or cultivation medium over the course of 1 day, 2 days, 3 days, 4 days, 5 days by means of a feeding solution;
iii) Adding at least one precursor and/or acceptor feed in a continuous manner to the culture or cultivation medium over the course of 1 day, 2 days, 3 days, 4 days, 5 days by means of a feeding solution and wherein preferably, the pH of the feeding solution is set between 3 and 7 and wherein preferably, the temperature of the feeding solution is kept between 20° C. and 80° C.;
   the method resulting in a saccharide with a concentration of at least 30 g/L in the final volume of the culture or cultivation medium.

In another and/or additional preferred embodiment of the method of the disclosure, the method for production of a saccharide as described herein comprises at least one of the following steps:
i) Adding to the culture or cultivation medium at least one precursor and/or acceptor in one pulse or in a discontinuous (pulsed) manner wherein the total reactor volume ranges from 250 mL (milliliter) to 10,000 m³ (cubic meter), preferably so that the final volume of the culture or cultivation medium is not more than three-fold, preferably not more than two-fold, more preferably less than two-fold of the volume of the culture or cultivation medium before the addition of the precursor and/or acceptor feed pulse(s);
ii) Adding at least one precursor and/or acceptor feed in a discontinuous (pulsed) manner to the culture or cultivation medium over the course of 5 minutes, 10 minutes, 30 minutes, 1 hour, 2 hours, 4 hours, 10 hours, 12 hours, 1 day, 2 days, 3 days, 4 days, 5 days by means of a feeding solution;
iii) Adding at least one precursor and/or acceptor feed in a discontinuous (pulsed) manner to the culture or cultivation medium over the course of 5 minutes, 10 minutes, 30 minutes, 1 hour, 2 hours, 4 hours, 10 hours, 12 hours, 1 day, 2 days, 3 days, 4 days, 5 days by means of a feeding solution and wherein preferably, the pH of the feeding solution is set between 3 and 7 and wherein preferably, the temperature of the feeding solution is kept between 20° C. and 80° C.;
   the method resulting in a saccharide with a concentration of at least 30 g/L in the final volume of the culture or cultivation medium.

In a further, more preferred embodiment, the method for the production of a saccharide as described herein comprises at least one of the following steps:
i) Adding to the culture medium a lactose feed comprising at least 50, more preferably at least 75, more preferably at least 100, more preferably at least 120, more preferably at least 150 gram of lactose per liter of initial reactor volume wherein the total reactor volume ranges from 250 mL (milliliter) to 10,000 m³ (cubic meter), preferably in a continuous manner, and preferably so that the final volume of the culture or cultivation medium is not more than three-fold, preferably not more than two-fold, more preferably less than two-fold of the volume of the culture or cultivation medium before the addition of the lactose feed;
ii) Adding a lactose feed in a continuous manner to the culture or cultivation medium over the course of 1 day, 2 days, 3 days, 4 days, 5 days by means of a feeding solution;
iii) Adding a lactose feed in a continuous manner to the culture or cultivation medium over the course of 1 day, 2 days, 3 days, 4 days, 5 days by means of a feeding solution and wherein the concentration of the lactose feeding solution is 25 g/L, preferably 50 g/L, more preferably 75 g/L, more preferably 100 g/L, more preferably 125 g/L, more preferably 150 g/L, more preferably 175 g/L, more preferably 200 g/L, more preferably 225 g/L, more preferably 250 g/L, more preferably 275 g/L, more preferably 300 g/L, more preferably 325 g/L, more preferably 350 g/L, more preferably 375 g/L, more preferably, 400 g/L, more preferably 450 g/L, more preferably 500 g/L, even more preferably, 550 g/L, most preferably 600 g/L; and wherein preferably the pH of the solution is set between 3 and 7 and
wherein preferably the temperature of the feed solution is kept between 20° C. and 80° C.;
   the method resulting in a saccharide with a concentration of at least 30 g/L in the final volume of the culture or cultivation medium.

Preferably the lactose feed is accomplished by adding lactose from the beginning of the cultivating in a concentration of at least 1 mM, preferably at least 5 mM, preferably in a concentration of 30, 40, 50, 60, 70, 80, 90, 100, 150 mM, more preferably in a concentration>300 mM.

In another aspect the lactose feed is accomplished by adding lactose to the cultivation medium in a concentration, such that throughout the production phase of the cultivation a lactose concentration of at least 1 mM, preferably 5 mM, 10 mM or 30 mM is obtained.

In a further embodiment of the methods described herein the cells are cultivated for at least about 60, 80, 100, or about 120 hours or in a continuous manner.

In a preferred embodiment, a carbon source is provided, preferably sucrose, in the culture or cultivation medium for 3 or more days, preferably up to 7 days; and/or provided, in the culture or cultivation medium, at least 100, advantageously at least 105, more advantageously at least 110, even more advantageously at least 120 grams of sucrose per liter of initial culture volume in a continuous manner, so that the final volume of the culture or cultivation medium is not more than three-fold, advantageously not more than two-fold, more advantageously less than two-fold of the volume of the culturing or cultivation medium before the culturing.

Preferably, when performing the method as described herein, a first phase of exponential cell growth is provided by adding a carbon source, preferably glucose or sucrose, to the culture or cultivation medium before the lactose is added to the culture or cultivation medium in a second phase.

In an alternative preferable embodiment, in the method as described herein, the lactose is added already in the first phase of exponential growth together with the carbon-based substrate.

In another and/or additional preferred embodiment of the method and/or cell of the disclosure, the cell comprises a catabolic pathway for selected mono-, di- or oligosaccharides that is at least partially inactivated, the mono-, di-, or oligosaccharides being involved in and/or required for the synthesis of the saccharide.

In another and/or additional preferred embodiment of the method and/or cell of the disclosure, the cell is selected from the list consisting of prokaryotic cells and eukaryotic cells, preferably from the list consisting of yeast cells, bacterial cells, archaebacterial cells, algae cells, plant cells and fungal cells. In another preferred embodiment, the cell is a bacterium, fungus, yeast or a plant cell.

The latter bacterium preferably belongs to the phylum of the Proteobacteria or the phylum of the Firmicutes or the phylum of the Cyanobacteria or the phylum Deinococcus-Thermus or the phylum of Actinobacteria. The latter bacterium belonging to the phylum Proteobacteria belongs preferably to the family Enterobacteriaceae, preferably to the species Escherichia coli. The latter bacterium preferably relates to any strain belonging to the species Escherichia coli such as but not limited to Escherichia coli B, Escherichia coli C, Escherichia coli W, Escherichia coli K12, Escherichia coli Nissle. More specifically, the latter term relates to cultivated Escherichia coli strains, designated as E. coli K12 strains, which are well-adapted to the laboratory environment, and, unlike wild-type strains, have lost their ability to thrive in the intestine. Well-known examples of the E. coli K12 strains are K12 Wild-type, W3110, MG1655, M182, MC1000, MC1060, MC1061, MC4100, JM101, NZN111 and AA200. Hence, the disclosure specifically relates to a mutated and/or transformed Escherichia coli cell or strain as indicated above wherein the E. coli strain is a K12 strain. More preferably, the Escherichia coli K12 strain is E. coli MG1655. The latter bacterium belonging to the phylum Firmicutes belongs preferably to the Bacilli, preferably Lactobacilliales, with members such as Lactobacillus lactis, Leuconostoc mesenteroides, or Bacillales with members such as from the genus Bacillus, such as Bacillus subtilis or, B. amyloliquefaciens. The latter Bacterium belonging to the phylum Actinobacteria, preferably belonging to the family of the Corynebacteriaceae, with members Corynebacterium glutamicum or C. afermentans, or belonging to the family of the Streptomycetaceae with members Streptomyces griseus or S. fradiae. The latter bacterium belonging to the phylum Proteobacteria, preferably belonging to the family of the Vibrionaceae, with member Vibrio natriegens. The latter yeast preferably belongs to the phylum of the Ascomycota or the phylum of the Basidiomycota or the phylum of the Deuteromycota or the phylum of the Zygomycetes. The latter yeast belongs preferably to the genus Saccharomyces (with members like, e.g., Saccharomyces cerevisiae, S. bayanus, S. boulardii), Zygosaccharomyces, Pichia (with members like, e.g., Pichia pastoris, P. anomala, P. kluyveri), Komagataella, Hansenula, Kluyveromyces (with members like, e.g., Kluyveromyces lactis, K. marxianus, K. thermotolerans), Debaromyces, Candida, Schizosaccharomyces, Schwanniomyces, Torulaspora, Yarrowia (like, e.g., Yarrowia lipolytica) or Starmerella (like, e.g., Starmerella bombicola). The latter yeast is preferably selected from Pichia pastoris, Yarrowia lipolytica, Saccharomyces cerevisiae, Kluyveromyces lactis, Hansenula polymorpha, Kluyveromyces marxianus, Pichia methanolica, Pichia stipites, Candida boidinii, Schizosaccharomyces pombe, Schwanniomyces occidentalis, Torulaspora delbrueckii, Zygosaccharomyces rouxii, and Zygosaccharomyces bailii. The latter fungus belongs preferably to the genus Rhizopus, Dictyostelium, Penicillium, Mucor or Aspergillus. The latter plant cell preferably is an algal cell or is derived from rose, tobacco, alfalfa, rice, tomato, cotton, rapeseed, soy, maize, or corn plant. More preferably, the latter plant cell is selected from the Rosa family.

In another and/or additional preferred embodiment, the cell is an E. coli or yeast with a lactose permease positive phenotype, preferably wherein the lactose permease is coded by the gene LacY or LAC12, respectively.

In another and/or additional preferred embodiment of the method and/or cell of the disclosure, the cell produces 30 g/L or more of the saccharide in the whole broth and/or supernatant and/or wherein the saccharide in the whole broth and/or supernatant has a purity of at least 80% measured on the total amount of saccharide and its precursor(s) produced by the cell in the whole broth and/or supernatant, respectively. In a more preferred embodiment, the cell produces 30 g/L, 31 g/L, 32 g/L, 33 g/L, 34 g/L, 35 g/L, 36 g/L, 37 g/L, 38 g/L, 39 g/L, 40 g/L, 41 g/L, 42 g/L, 43 g/L, 44 g/L, 45 g/L, 46 g/L, 47 g/L, 48 g/L, 49 g/L, 50 g/L, 51 g/L, 52 g/L, 53 g/L, 54 g/L, 55 g/L, 56 g/L, 57 g/L, 58 g/L, 59 g/L, 60 g/L, 61 g/L, 62 g/L, 63 g/L, 64 g/L, 65 g/L, 66 g/L, 67 g/L, 68 g/L, 69 g/L, 70 g/L, 71 g/L, 72 g/L, 73 g/L, 74 g/L, 75 g/L, 76 g/L, 77 g/L, 78 g/L, 79 g/L, 80 g/L, 81 g/L, 82 g/L, 83 g/L, 84 g/L, 85 g/L or more than 85 g/L of the saccharide in the whole broth and/or supernatant.

In another and/or additional preferred embodiment of the method and/or cell of the disclosure, the less functional or knocked out synthesis of LBA, a modified form of LBA, a glycosylated form of LBA, fucosylated LBA and/or sialylated LBA confers unaffected and/or enhanced i) saccharide formation, ii) productivity, iii) biomass production, iv) cell growth and/or v) yield of the produced saccharide, relative to a corresponding non-modified cell.

In another aspect of the disclosure, the cell produces a saccharide as described herein.

In a preferred embodiment of the method and/or cell of the disclosure, the cell produces a mixture comprising, consisting of or consisting essentially of a saccharide as described herein and any one or more of LBA, a modified form of LBA, a glycosylated form of LBA, fucosylated LBA and/or sialylated LBA. In another and/or additional preferred embodiment, the cell produces a mixture comprising, consisting of or consisting essentially of a saccharide as described herein and any one or more of LBA, a modified form of LBA, a glycosylated form of LBA, fucosylated LBA and/or sialylated LBA wherein the mixture comprises≤10 weight % of the LBA, modified form of LBA, glycosylated form of LBA, fucosylated LBA and/or sialylated LBA; preferably ≤9 weight % of the LBA, modified form of LBA, glycosylated form of LBA, fucosylated LBA and/or sialylated LBA; more preferably ≤8 weight % of the LBA, modified form of LBA, glycosylated form of LBA, fucosylated LBA and/or sialylated LBA; even more preferably ≤7 weight % of the LBA, modified form of LBA, glycosylated form of LBA, fucosylated LBA and/or sialylated LBA; even more preferably ≤6 weight % of the LBA, modified form of LBA, glycosylated form of LBA, fucosylated LBA and/or sialylated LBA; even more preferably ≤5 weight % of the LBA, modified form of LBA, glycosylated form of LBA, fucosylated LBA and/or sialylated LBA; even more preferably ≤4 weight % of the LBA, modified form of LBA, glycosylated form of LBA, fucosylated LBA and/or sialylated LBA; even more preferably ≤3 weight % of the LBA, modified form of LBA, glycosylated form of LBA, fucosylated LBA and/or sialylated LBA; even more preferably ≤2 weight % of the LBA, modified form of LBA, glycosylated form of LBA, fucosylated LBA and/or sialylated LBA; even more preferably ≤1 weight % of the LBA, modified form of LBA, glycosylated form of LBA, fucosylated LBA and/or sialylated LBA; even more preferably ≤0.5 weight % of the LBA, modified form of LBA, glycosylated form of LBA, fucosylated LBA and/or sialylated LBA; most preferably ≤0.1 weight % of the LBA, modified form of LBA, glycosylated form of LBA, fucosylated LBA and/or sialylated LBA.

In another and/or additional preferred embodiment, the cell produces no LBA.

In another aspect of the disclosure, the saccharide produced by a cell of the disclosure is recovered from the cultivation or incubation medium and/or the cell. In a preferred embodiment, the saccharide is purified. In another and/or additional preferred embodiment, the saccharide is purified from the LBA. In another and/or additional preferred embodiment, the saccharide is purified from the modified form of LBA, glycosylated form of LBA, fucosylated LBA and/or sialylated LBA.

The terms “separating from the cultivation or incubation” means harvesting, collecting, or retrieving the saccharide from the cell and/or the medium of its growth.

The saccharide can be separated in a conventional manner from the aqueous culture or cultivation medium, in which the cell was grown. In case the saccharide is still present in the cells producing the saccharide, conventional manners to free or to extract the saccharide out of the cells can be used, such as cell destruction using high pH, heat shock, sonication, French press, homogenization, enzymatic hydrolysis, chemical hydrolysis, solvent hydrolysis, detergent, hydrolysis, etc. The culture or cultivation medium and/or cell extract together and separately can then be further used for separating the saccharide.

This preferably involves clarifying the saccharide to remove suspended particulates and contaminants, particularly cells, cell components, insoluble metabolites and debris produced by culturing the genetically engineered cell. In this step, the saccharide can be clarified in a conventional manner. Preferably, the saccharide is clarified by centrifugation, flocculation, decantation and/or filtration. A second step of separating the saccharide preferably involves removing substantially all the eventually remaining proteins, peptides, amino acids, RNA, DNA, endotoxins and glycolipids that could interfere with the subsequent separation step, from the saccharide, preferably after it has been clarified. In this step, remaining proteins and related impurities can be removed from the saccharide in a conventional manner. Preferably, remaining proteins, salts, by-products, color, endotoxins and other related impurities are removed from the saccharide by ultrafiltration, nanofiltration, two-phase partitioning, reverse osmosis, microfiltration, activated charcoal or carbon treatment, treatment with non-ionic surfactants, enzymatic digestion, tangential flow high-performance filtration, tangential flow ultrafiltration, electrophoresis (e.g., using slab-polyacrylamide or sodium dodecyl sulphate-polyacrylamide gel electrophoresis (PAGE)), affinity chromatography (using affinity ligands including, e.g., DEAE-sepharose, poly-L-lysine and polymyxin-B, endotoxin-selective adsorber matrices), ion exchange chromatography (such as but not limited to cation exchange, anion exchange, mixed bed ion exchange, inside-out ligand attachment), hydrophobic interaction chromatography and/or gel filtration (i.e., size exclusion chromatography), particularly by chromatography, more particularly by ion exchange chromatography or hydrophobic interaction chromatography or ligand exchange chromatography. With the exception of size exclusion chromatography, remaining proteins and related impurities are retained by a chromatography medium or a selected membrane.

In a further preferred embodiment, the methods as described herein also provide for a further purification of the saccharide of the disclosure. A further purification of the saccharide may be accomplished, for example, by use of (activated) charcoal or carbon, nanofiltration, ultrafiltration, electrophoresis, enzymatic treatment or ion exchange to remove any remaining DNA, protein, LPS, endotoxins, or other impurity. Alcohols, such as ethanol, and aqueous alcohol mixtures can also be used. Another purification step is accomplished by crystallization, evaporation or precipitation of the saccharide. Another purification step is to dry, e.g., spray dry or lyophilize the produced saccharide.

In an exemplary embodiment, the separation and purification of the saccharide is made in a process, comprising the following steps in any order:
a) contacting the cultivation or a clarified version thereof with a nanofiltration membrane with a molecular weight cut-off (MWCO) of 600-3500 Da ensuring the retention of the produced saccharide and allowing at least a part of the proteins, salts, by-products, color and other related impurities to pass,
b) conducting a diafiltration process on the retentate from step a), using the membrane, with an aqueous solution of an inorganic electrolyte, followed by optional diafiltration with pure water to remove excess of the electrolyte, and
c) collecting the retentate enriched in the saccharide in the form of a salt from the cation of the electrolyte.

In an alternative exemplary embodiment, the separation and purification of the saccharide is made in a process, comprising the following steps in any order: subjecting the cultivation or a clarified version thereof to two membrane filtration steps using different membranes, wherein
one membrane has a molecular weight cut-off of between about 300 to about 500 Dalton, and
the other membrane as a molecular weight cut-off of between about 600 to about 800 Dalton.

In an alternative exemplary embodiment, the separation and purification of the saccharide is made in a process, comprising the following steps in any order comprising the step of treating the cultivation or a clarified version thereof with a strong cation exchange resin in H+-form and a weak anion exchange resin in free base form.

In an alternative exemplary embodiment, the separation and purification of the saccharide is made in the following way. The cultivation comprising the produced saccharide, biomass, medium components and contaminants, and wherein the purity of the produced saccharide in the cultivation is <80%, is applied to the following purification steps:
i) separation of biomass from the cultivation,
ii) cationic ion exchanger treatment for the removal of positively charged material,
iii) anionic ion exchanger treatment for the removal of negatively charged material,
iv) nanofiltration step and/or electrodialysis step,
wherein a purified solution comprising the produced saccharide at a purity of greater than or equal to 80% is provided. Optionally the purified solution is spray dried.

In an alternative exemplary embodiment, the separation and purification of the saccharide is made in a process, comprising the following steps in any order: enzymatic treatment of the cultivation; removal of the biomass from the cultivation; ultrafiltration; nanofiltration; and a column chromatography step. Preferably, such column chromatography is a single column or a multiple column. Further preferably, the column chromatography step is simulated moving bed chromatography. Such simulated moving bed chromatography preferably comprises i) at least 4 columns, wherein at least one column comprises a weak or strong cation exchange resin; and/or ii) four zones I, II, III and IV with different flow rates; and/or iii) an eluent comprising water; and/or iv) an operating temperature of 15 degrees to 60 degrees centigrade.

In a specific embodiment, the disclosure provides the produced saccharide that is spray-dried to powder, wherein the spray-dried powder contains<15%-wt. of water, preferably <10%-wt. of water, more preferably <7%-wt. of water, most preferably <5%-wt. of water.

For identification of the saccharide as described herein, the monomeric building blocks (e.g., the monosaccharide or glycan unit composition), the anomeric configuration of side chains, the presence and location of substituent groups, degree of polymerization/molecular weight and the linkage pattern can be identified by standard methods known in the art, such as, e.g., methylation analysis, reductive cleavage, hydrolysis, GC-MS (gas chromatography-mass spectrometry), MALDI-MS (Matrix-assisted laser desorption/ionization-mass spectrometry), ESI-MS (Electrospray ionization-mass spectrometry), HPLC (High-Performance Liquid chromatography with ultraviolet or refractive index detection), HPAEC-PAD (High-Performance Anion-Exchange chromatography with Pulsed Amperometric Detection), CE (capillary electrophoresis), IR (infrared)/Raman spectroscopy, and NMR (Nuclear magnetic resonance) spectroscopy techniques. The crystal structure can be solved using, e.g., solid-state NMR, FT-IR (Fourier transform infrared spectroscopy), and WAXS (wide-angle X-ray scattering). The degree of polymerization (DP), the DP distribution, and polydispersity can be determined by, e.g., viscosimetry and SEC (SEC-HPLC, high performance size-exclusion chromatography). To identify the monomeric components of the saccharide methods such as, e.g., acid-catalyzed hydrolysis, HPLC (high performance liquid chromatography) or GLC (gas-liquid chromatography) (after conversion to alditol acetates) may be used. To determine the glycosidic linkages, the saccharide is methylated with methyl iodide and strong base in DMSO, hydrolysis is performed, a reduction to partially methylated alditols is achieved, an acetylation to methylated alditol acetates is performed, and the analysis is carried out by GLC/MS (gas-liquid chromatography coupled with mass spectrometry). To determine the glycan sequence, a partial depolymerization is carried out using an acid or enzymes to determine the structures. To identify the anomeric configuration, the saccharide is subjected to enzymatic analysis, e.g., it is contacted with an enzyme that is specific for a particular type of linkage, e.g., 0-galactosidase, or α-glucosidase, etc., and NMR may be used to analyze the products.

In another aspect, the disclosure provides use of a cell as described herein for the production of a saccharide wherein the saccharide is selected from the list comprising, consisting of or consisting essentially of monosaccharide, phosphorylated monosaccharide, activated monosaccharide, disaccharide, oligosaccharide, neutral (non-charged) oligosaccharide, a negatively charged, preferably sialylated, oligosaccharide, milk oligosaccharide, preferably a mammalian milk oligosaccharide (MMO), more preferably a human milk oligosaccharide (HMO); sialylated milk oligosaccharide, neutral (non-charged) milk oligosaccharide, fucosylated milk oligosaccharide, non-fucosylated neutral (non-charged) milk oligosaccharide, sialylated mammalian milk oligosaccharide, neutral (non-charged) mammalian milk oligosaccharide, fucosylated mammalian milk oligosaccharide, non-fucosylated neutral (non-charged) mammalian milk oligosaccharide, sialylated human milk oligosaccharide, neutral (non-charged) human milk oligosaccharide, fucosylated human milk oligosaccharide, non-fucosylated neutral (non-charged) human milk oligosaccharide; O-antigen; enterobacterial common antigen (ECA); the oligosaccharide repeats present in capsular polysaccharides; peptidoglycan; an amino-sugar; Lewis-type antigen oligosaccharide; an antigen of the human ABO blood group system; an animal oligosaccharide, preferably selected from the list consisting of N-glycans and O-glycans; a plant oligosaccharide, preferably selected from the list consisting of N-glycans and O-glycans; fucosylated oligosaccharide, preferably selected from the list comprising, consisting of or consisting essentially of 2′-fucosyllactose (2′FL), 3-fucosyllactose (3FL), 4-fucosyllactose (4FL), 6-fucosyllactose (6FL), 2′,3-difucosyllactose (diFL), lacto-N-fucopentaose I, lacto-N-neofucopentaose I, lacto-N-fucopentaose II, lacto-N-fucopentaose III, lacto-N-fucopentaose V, lacto-N-fucopentaose VI, lacto-N-neofucopentaose V, lacto-N-difucohexaose I, lacto-N-difucohexaose II, difucosyl-lacto-N-hexaose and difucosyl-lacto-N-neohexaose; sialylated oligosaccharide, preferably selected from the list comprising, consisting of or consisting essentially of 3′sialyllactose (3′SL), 6′sialyllactose (6′SL), sialyllacto-N-tetraose a (LSTa), sialyllacto-N-tetraose b (LSTb), sialyllacto-N-tetraose c (LSTc), sialyllacto-N-tetraose d (LSTd), disialyllacto-N-tetraose, disialyllacto-N-neotetraose, monosialyllacto-N-hexaose, disialyllacto-N-hexaose I, disialyllacto-N-hexaose II, monosialyllacto-N-neohexaose I, monosialyllacto-N-neohexaose II, disialyllacto-N-neohexaose, 3′-sialyl-3-fucosyllactose, fucodisialyllacto-N-hexaose, disialomonofucosyllacto-N-neohexaose, sialyllacto-N-fucohexaose II, disialyllacto-N-fucopentaose II and monofucosyldisialyllacto-N-tetraose; N-acetylglucosamine containing neutral (non-charged) saccharide, preferably selected from the list comprising, consisting of or consisting essentially of lacto-N-biose (LNB), N-acetyllactosamine (LacNAc), lacto-N-triose II (LN3), lacto-N-tetraose (LNT), lacto-N-neotetraose (LNnT), 6′-galactosyllactose, 3′-galactosyllactose, lacto-N-hexaose, lacto-N-neohexaose, para-lacto-N-hexaose, para-lacto-N-neohexaose; N-acetylglucosamine containing saccharide; N-acetyllactosamine containing saccharide; lacto-N-biose containing saccharide; non-fucosylated neutral (non-charged) saccharide; chitosan; chitosan comprising oligosaccharide; heparosan; chondroitin sulphate; glycosaminoglycan oligosaccharide; heparin; heparan sulphate; dermatan sulphate; hyaluronan; hyaluronic acid; and keratan sulphate.

In another aspect, the disclosure provides use of a method as described herein for the production of a saccharide, wherein the saccharide is selected from the list comprising, consisting of or consisting essentially of monosaccharide, phosphorylated monosaccharide, activated monosaccharide, disaccharide, oligosaccharide, neutral (non-charged) oligosaccharide, a negatively charged, preferably sialylated, oligosaccharide, milk oligosaccharide, preferably a mammalian milk oligosaccharide (MMO), more preferably a human milk oligosaccharide (HMO); sialylated milk oligosaccharide, neutral (non-charged) milk oligosaccharide, fucosylated milk oligosaccharide, non-fucosylated neutral (non-charged) milk oligosaccharide, sialylated mammalian milk oligosaccharide, neutral (non-charged) mammalian milk oligosaccharide, fucosylated mammalian milk oligosaccharide, non-fucosylated neutral (non-charged) mammalian milk oligosaccharide, sialylated human milk oligosaccharide, neutral (non-charged) human milk oligosaccharide, fucosylated human milk oligosaccharide, non-fucosylated neutral (non-charged) human milk oligosaccharide; O-antigen; enterobacterial common antigen (ECA); the oligosaccharide repeats present in capsular polysaccharides; peptidoglycan; an amino-sugar; Lewis-type antigen oligosaccharide; an antigen of the human ABO blood group system; an animal oligosaccharide, preferably selected from the list consisting of N-glycans and O-glycans; a plant oligosaccharide, preferably selected from the list consisting of N-glycans and O-glycans; fucosylated oligosaccharide, preferably selected from the list comprising, consisting of or consisting essentially of 2′-fucosyllactose (2′FL), 3-fucosyllactose (3FL), 4-fucosyllactose (4FL), 6-fucosyllactose (6FL), 2′,3-difucosyllactose (diFL), lacto-N-fucopentaose I, lacto-N-neofucopentaose I, lacto-N-fucopentaose II, lacto-N-fucopentaose III, lacto-N-fucopentaose V, lacto-N-fucopentaose VI, lacto-N-neofucopentaose V, lacto-N-difucohexaose I, lacto-N-difucohexaose II, difucosyl-lacto-N-hexaose and difucosyl-lacto-N-neohexaose; sialylated oligosaccharide, preferably selected from the list comprising, consisting of or consisting essentially of 3′sialyllactose (3′SL), 6′sialyllactose (6′SL), sialyllacto-N-tetraose a (LSTa), sialyllacto-N-tetraose b (LSTb), sialyllacto-N-tetraose c (LSTc), sialyllacto-N-tetraose d (LSTd), disialyllacto-N-tetraose, disialyllacto-N-neotetraose, monosialyllacto-N-hexaose, disialyllacto-N-hexaose I, disialyllacto-N-hexaose II, monosialyllacto-N-neohexaose I, monosialyllacto-N-neohexaose II, disialyllacto-N-neohexaose, 3′-sialyl-3-fucosyllactose, fucodisialyllacto-N-hexaose, disialomonofucosyllacto-N-neohexaose, sialyllacto-N-fucohexaose II, disialyllacto-N-fucopentaose II and monofucosyldisialyllacto-N-tetraose; N-acetylglucosamine containing neutral (non-charged) saccharide, preferably selected from the list comprising, consisting of or consisting essentially of lacto-N-biose (LNB), N-acetyllactosamine (LacNAc), lacto-N-triose II (LN3), lacto-N-tetraose (LNT), lacto-N-neotetraose (LNnT), 6′-galactosyllactose, 3′-galactosyllactose, lacto-N-hexaose, lacto-N-neohexaose, para-lacto-N-hexaose, para-lacto-N-neohexaose; N-acetylglucosamine containing saccharide; N-acetyllactosamine containing saccharide; lacto-N-biose containing saccharide; non-fucosylated neutral (non-charged) saccharide; chitosan; chitosan comprising oligosaccharide; heparosan; chondroitin sulphate; glycosaminoglycan oligosaccharide; heparin; heparan sulphate; dermatan sulphate; hyaluronan; hyaluronic acid; and keratan sulphate.

In another aspect, the disclosure provides for a purified saccharide or a purified saccharide mixture as described herein for use in medicine, preferably for use in prophylaxis or therapy of a gastrointestinal disorder.

In another aspect, the disclosure provides use of a purified saccharide obtained by a method as described herein in a food or feed preparation, in a dietary supplement, in a cosmetic ingredient or in a pharmaceutical ingredient. In some embodiments, the saccharide is mixed with one or more ingredients suitable for food, feed, dietary supplement, pharmaceutical ingredient, cosmetic ingredient or medicine. The purified saccharide may be used for the manufacture of a preparation, as food additive, prebiotic, symbiotic, for the supplementation of baby food, adult food, infant animal feed, adult animal feed, or as either therapeutically or pharmaceutically active compound or in cosmetic applications. In another aspect, the disclosure provides use of saccharide or saccharide mixture as described herein as additive in food, preferably as additive in human food and/or pet food, more preferably as additive in human baby food. In the context of the disclosure, the food is a human food, preferably infant food, human baby food and/or an infant formula or an infant supplement and the feed is a pet food, animal milk replacer, veterinary product, veterinary feed supplement, nutrition supplement, post weaning feed, or creep feed.

In another preferred embodiment, a preparation is provided that further comprises at least one probiotic microorganism. In another preferred embodiment of the disclosure, preparation is a nutritional composition. In a more preferred embodiment, the preparation is a medicinal formulation, a dietary supplement, a dairy drink or an infant formula. A “prebiotic” is a substance that promotes growth of microorganisms beneficial to the host, particularly microorganisms in the gastrointestinal tract. In some embodiments, a dietary supplement provides multiple prebiotics, including the saccharide being a prebiotic purified by a method disclosed in this specification, to promote growth of one or more beneficial microorganisms. Examples of prebiotic ingredients for dietary supplements include other prebiotic molecules (such as HMOs) and plant polysaccharides (such as inulin, pectin, b-glucan and xylooligosaccharide). A “probiotic” product typically contains live microorganisms that replace or add to gastrointestinal microflora, to the benefit of the recipient. Examples of such microorganisms include Lactobacillus species (for example, L. acidophilus and L. bulgaricus), Bifidobacterium species (for example, B. animalis, B. longum and B. infantis (e.g., Bi-26)), and Saccharomyces boulardii. In some embodiments, the saccharide produced and/or purified by a method of this specification is orally administered in combination with such microorganism. Examples of further ingredients for dietary supplements include oligosaccharides (such as 2′-fucosyllactose, 3-fucosyllactose, 3′-sialyllactose, 6′-sialyllactose), disaccharides (such as lactose), monosaccharides (such as glucose, galactose, L-fucose, sialic acid, glucosamine and N-acetylglucosamine), thickeners (such as gum arabic), acidity regulators (such as trisodium citrate), water, skimmed milk, and flavorings.

In some embodiments, the saccharide purified by a method as described herein is incorporated into a human baby food (e.g., infant formula). Infant formula is generally a manufactured food for feeding to infants as a complete or partial substitute for human breast milk. In some embodiments, infant formula is sold as a powder and prepared for bottle- or cup-feeding to an infant by mixing with water. The composition of infant formula is typically designed to be roughly mimic human breast milk. In some embodiments, the saccharide purified by a method as described herein is included in infant formula to provide nutritional benefits similar to those provided by the oligosaccharides in human breast milk. In some embodiments, the purified saccharide is mixed with one or more ingredients of the infant formula. Examples of infant formula ingredients include non-fat milk, carbohydrate sources (e.g., lactose), protein sources (e.g., whey protein concentrate and casein), fat sources (e.g., vegetable oils—such as palm, high oleic safflower oil, rapeseed, coconut and/or sunflower oil; and fish oils), vitamins (such as vitamins A, Bb, Bi2, C and D), minerals (such as potassium citrate, calcium citrate, magnesium chloride, sodium chloride, sodium citrate and calcium phosphate) and possibly human milk oligosaccharides (HMOs). In some embodiments, the one or more infant formula ingredients comprise non-fat milk, a carbohydrate source, a protein source, a fat source, and/or a vitamin and mineral. In some embodiments, the one or more infant formula ingredients comprise lactose, whey protein concentrate and/or high oleic safflower oil. In some embodiments, the concentration of the oligosaccharide in the infant formula is approximately the same concentration as the concentration of the oligosaccharide generally present in human breast milk. In some embodiments, a saccharide purified by a method as described herein is added to the infant formula with a concentration that is approximately the same concentration as the concentration of the compound generally present in human breast milk.

In another aspect, the present application provides for a mixture comprising, consisting of or consisting essentially of (i) a saccharide and (ii) LBA, a modified form of LBA, a glycosylated form of LBA, fucosylated LBA and/or sialylated LBA wherein the saccharide, LBA, modified form of LBA, glycosylated form of LBA, fucosylated LBA and/or sialylated LBA are obtainable or obtained by a method as described herein.

In another and/or additional specific embodiment, the present application provides for a mixture comprising, consisting of or consisting essentially of (i) a saccharide and (ii) ≤10 weight % LBA, 9 weight % LBA, 8 weight % LBA, 7 weight % LBA, ≤6 weight % LBA, 5 weight % LBA, 4 weight % LBA, 3 weight % LBA, 2 weight % LBA, ≤1 weight % LBA, ≤0.5 weight % LBA and/or ≤0.1 weight % LBA, wherein the saccharide and LBA are obtainable or obtained by a method as described herein.

In another and/or additional specific embodiment, the present application provides for a mixture comprising, consisting of or consisting essentially of a saccharide and (i) ≤10 weight % LBA, ≤9 weight % LBA, ≤8 weight % LBA, ≤7 weight % LBA, ≤6 weight % LBA, ≤5 weight % LBA, ≤4 weight % LBA, ≤3 weight % LBA, 2 weight % LBA, ≤1 weight % LBA, ≤0.5 weight % LBA and/or 0.1 weight % LBA and/or (ii)≤10 weight % of a modified form of LBA, a glycosylated form of LBA, fucosylated LBA and/or sialylated LBA; ≤9 weight % of a modified form of LBA, a glycosylated form of LBA, fucosylated LBA and/or sialylated LBA; ≤8 weight % of a modified form of LBA, a glycosylated form of LBA, fucosylated LBA and/or sialylated LBA; ≤7 weight % of a modified form of LBA, a glycosylated form of LBA, fucosylated LBA and/or sialylated LBA; ≤6 weight % of a modified form of LBA, a glycosylated form of LBA, fucosylated LBA and/or sialylated LBA; ≤5 weight % of a modified form of LBA, a glycosylated form of LBA, fucosylated LBA and/or sialylated LBA; ≤4 weight % of a modified form of LBA, a glycosylated form of LBA, fucosylated LBA and/or sialylated LBA; ≤2 weight % of a modified form of LBA, a glycosylated form of LBA, fucosylated LBA and/or sialylated LBA; ≤1 weight % of a modified form of LBA, a glycosylated form of LBA, fucosylated LBA and/or sialylated LBA; ≤0.5 weight % of a modified form of LBA, a glycosylated form of LBA, fucosylated LBA and/or sialylated LBA and/or ≤0.1 weight % of a modified form of LBA, a glycosylated form of LBA, fucosylated LBA and/or sialylated LBA, wherein the saccharide, LBA, modified form of LBA, glycosylated form of LBA, fucosylated LBA and/or sialylated LBA are obtainable or obtained by a method as described herein.

Unless defined otherwise, all technical and scientific terms used herein generally have the same meaning as commonly understood by one of ordinary skill in the art to which this disclosure belongs. Generally, the nomenclature used herein and the laboratory procedures in cell culture, molecular genetics, organic chemistry and nucleic acid chemistry and hybridization described above and below are those well-known and commonly employed in the art. Standard techniques are used for nucleic acid and peptide synthesis. Generally, purification steps are performed according to the manufacturer's specifications.

Further advantages follow from the specific embodiments and the examples. It goes without saying that the abovementioned features and the features that are still to be explained below can be used not only in the respectively specified combinations, but also in other combinations or on their own, without departing from the scope of the disclosure.

Moreover, the disclosure relates to the following specific embodiments:
1. A cell capable of synthesizing, preferably synthesizing, lactobionic acid (4-O-b-galactopyranosyl-D-gluconic acid, LBA), the cell genetically engineered for the production of a saccharide, the cell comprising a pathway for production of the saccharide, characterized in that LBA synthesis in the cell is rendered less functional or is knocked out.
2. Cell according to embodiment 1, wherein the pathway for production of the saccharide is chosen from the list comprising fucosylation pathway, sialylation pathway, galactosylation pathway, N-acetylglucosaminylation pathway, N-acetylgalactosaminylation pathway, mannosylation pathway and N-acetylmannosaminylation pathway, preferably the cell is genetically engineered to comprise at least one of the pathway(s), more preferably the cell comprises at least one of the pathway(s) wherein at least one of the pathway(s) has/have been genetically engineered.
3. Cell according to any one of embodiment 1 or 2, wherein the cell:
   possesses, preferably expresses, more preferably overexpresses, one or more glycosyltransferase(s) chosen from the list comprising fucosyltransferases, sialyltransferases, galactosyltransferases, glucosyltransferases, mannosyltransferases, N-acetylglucosaminyltransferases, N-acetylgalactosaminyltransferases, N-acetylmannosaminyltransferases, xylosyltransferases, glucuronyltransferases, galacturonyltransferases, glucosaminyltransferases, N-glycolylneuraminyltransferases, rhamnosyltransferases, N-acetylrhamnosyltransferases, UDP-4-amino-4,6-dideoxy-N-acetyl-β-L-altrosamine transaminases, UDP-N-acetylglucosamine enolpyruvyl transferases and fucosaminyltransferases, and/or
   is capable of producing, preferably produces, one or more nucleotide-activated sugars, preferably the cell is genetically engineered for production of one or more of the nucleotide-activated sugar(s).
4. Cell according to any one of previous embodiments, wherein the cell comprises a pathway for the synthesis of a nucleotide-activated sugar chosen from the list comprising UDP-N-acetylglucosamine (UDP-GlcNAc), UDP-N-acetylgalactosamine (UDP-GalNAc), UDP-N-acetylmannosamine (UDP-ManNAc), UDP-glucose (UDP-Glc), UDP-galactose (UDP-Gal), GDP-mannose (GDP-Man), GDP-fucose, (GDP-Fuc), UDP-glucuronate, UDP-galacturonate, UDP-2-acetamido-2,6-dideoxy-L-arabino-4-hexulose, UDP-2-acetamido-2,6-dideoxy-L-lyxo-4-hexulose, UDP-N-acetyl-L-rhamnosamine (UDP-L-RhaNAc or UDP-2-acetamido-2,6-dideoxy-L-mannose), dTDP-N-acetylfucosamine, UDP-N-acetylfucosamine (UDP-L-FucNAc or UDP-2-acetamido-2,6-dideoxy-L-galactose), UDP-N-acetyl-L-pneumosamine (UDP-L-PneNAC or UDP-2-acetamido-2,6-dideoxy-L-talose), UDP-N-acetylmuramic acid, UDP-N-acetyl-L-quinovosamine (UDP-L-QuiNAc or UDP-2-acetamido-2,6-dideoxy-L-glucose), CMP-sialic acid (CMP-Neu5Ac), CMP-Neu4Ac, CMP-Neu5Ac9N3, CMP-Neu4,5Ac2, CMP-Neu5,7Ac2, CMP-Neu5,9Ac2, CMP-Neu5,7(8,9)Ac2, CMP-N-glycolylneuraminic acid (CMP-Neu5Gc), GDP-rhamnose and UDP-xylose.
5. Cell according to any one of previous embodiments, wherein the cell possesses, preferably expresses, more preferably overexpresses, one or more genes chosen from the list comprising mannose-6-phosphate isomerase, phosphomannomutase, mannose-1-phosphate guanylyltransferase, GDP-mannose 4,6-dehydratase, GDP-L-fucose synthase, fucose permease, fucose kinase, fucose-1-phosphate guanylyltransferase, L-glutamine-D-fructose-6-phosphate aminotransferase, phosphoglucosamine mutase, N-acetylglucosamine-6-P deacetylase, N-acylglucosamine 2-epimerase, UDP-N-acetylglucosamine 2-epimerase, N-acetylmannosamine-6-phosphate 2-epimerase, UDP-GlcNAc 2-epimerase/kinase, glucosamine 6-phosphate N-acetyltransferase, N-acetylglucosamine-6-phosphate phosphatase, phosphoacetylglucosamine mutase, N-acetylglucosamine 1-phosphate uridylyltransferase, glucosamine-1-phosphate acetyltransferase, Neu5Ac synthase, N-acetylneuraminate lyase, N-acylneuraminate-9-phosphate synthase, N-acylneuraminate-9-phosphatase, sialic acid transporter, CMP kinase, CMP-sialic acid synthase, galactose-1-epimerase, galactokinase, glucokinase, galactose-1-phosphate uridylyltransferase, UDP-glucose 4-epimerase, glucose-1-phosphate uridylyltransferase, phosphoglucomutase, UDP-N-acetylglucosamine 4-epimerase, N-acetylgalactosamine kinase and UDP-N-acetylgalactosamine pyrophosphorylase.
6. Cell according to any one of previous embodiments, wherein the LBA synthesis is obtained by expression of at least one gene chosen from the list comprising genes encoding carbohydrate oxidase, dehydrogenase, lactonase, oxygen-dependent FAD-linked oxidoreductase, and pyrroloquinoline quinone (PQQ) oxidoreductase, and wherein the at least one gene is rendered less functional or knocked out.
7. Cell according to embodiment 6, wherein the:
   dehydrogenase is chosen from the list comprising lactose dehydrogenase, quinoprotein glucose dehydrogenase, aldose sugar dehydrogenase, glucose/fructose dehydrogenase, glucose/sorbosone dehydrogenase, cellobiose dehydrogenase, pyrroloquinoline quinone (PQQ) dehydrogenase, and malate dehydrogenase, and/or
   carbohydrate oxidase is a lactose oxidase.
8. Cell according to embodiment 6 or 7, wherein the at least one gene encodes an enzyme, wherein the enzyme:
   is chosen from an enzyme class chosen from the list comprising EC:1.1.3.-, EC:1.1.3.5, EC:1.1.3.4, EC:1.1.5.-, EC:1.1.5.2, and EC:1.1.99.18,
   comprises a polypeptide sequence comprising an IPR domain chosen from the list comprising IPR000172, IPR001479, IPR002372, IPR006094, IPR007867, IPR011041, IPR011042, IPR011047, IPR012132, IPR012938, 1PR012951, IPR013154, IPR013428, IPR015402, IPR015920, IPR016166, IPR016169, 1PR017511, IPR017512, IPR018391, IPR027424, IPR029056, IPR031640, IPR036188 and IPR036318 as defined by InterPro 90.0 as released on 4 Aug. 2022,
   comprises a polypeptide sequence comprising a Panther domain chosen from the list comprising PTHR11552, PTHR13460, PTHR32303:SF4, PTHR42973 and PTHR47190 as defined by InterPro 90.0 as released on 4 Aug. 2022,
   comprises a polypeptide sequence comprising a PFAM domain chosen from the list comprising PF00732, PF01011, PF05199, PF07995, PF08031, PF08240, PF13360, PF13570, PF01565, PF16010 and PF16912 as defined by InterPro 90.0 as released on 4 Aug. 2022,
   comprises a polypeptide sequence comprising the conserved protein domain cd10280 as defined by InterPro 90.0 as released on 4 Aug. 2022,
   is part of a NOG family chosen from the list comprising COG0277, COG2133, COG2303 and COG4993 as defined by eggNOG5.0 as released in 2019 and/or
   uses a cofactor chosen from the list comprising pyrroloquinoline quinone (PQQ), FAD, a divalent metal cation, Ca²⁺, Cu²⁺, Mg²⁺, Zn²⁺, heme, heme C and pyridoxal 5′-phosphate.
9. Cell according to any one of previous embodiments, wherein at least one gene involved in the synthesis and/or import of a co-factor that is involved in LBA synthesis is rendered less functional or knocked out, preferably the co-factor is chosen from the list comprising pyrroloquinoline quinone (PQQ), FAD, a divalent metal cation, Ca²⁺, Cu²⁺, Mg²⁺, Zn²⁺, heme, heme C and pyridoxal 5′-phosphate.
10. Cell according to any one of embodiments 6 to 9, wherein the at least one gene is rendered less functional by insertion, deletion and/or modification of one or more nucleotide(s) in one or more polynucleotide sequence(s) chosen from the list comprising promoter sequence, ribosome binding site, untranslated region, coding sequence and transcription terminator sequence of the at least one gene.
11. Cell according to any one of previous embodiments, wherein the saccharide is chosen from the list comprising monosaccharide, phosphorylated monosaccharide, activated monosaccharide, disaccharide, oligosaccharide, neutral (non-charged) oligosaccharide, a negatively charged, preferably sialylated, oligosaccharide, milk oligosaccharide, preferably a mammalian milk oligosaccharide (MMO), more preferably a human milk oligosaccharide (HMO); sialylated milk oligosaccharide, neutral (non-charged) milk oligosaccharide, fucosylated milk oligosaccharide, non-fucosylated neutral (non-charged) milk oligosaccharide, sialylated mammalian milk oligosaccharide, neutral (non-charged) mammalian milk oligosaccharide, fucosylated mammalian milk oligosaccharide, non-fucosylated neutral (non-charged) mammalian milk oligosaccharide, sialylated human milk oligosaccharide, neutral (non-charged) human milk oligosaccharide, fucosylated human milk oligosaccharide, non-fucosylated neutral (non-charged) human milk oligosaccharide; O-antigen; enterobacterial common antigen (ECA); the oligosaccharide repeats present in capsular polysaccharides; peptidoglycan; an amino-sugar; Lewis-type antigen oligosaccharide; an antigen of the human ABO blood group system; an animal oligosaccharide, preferably selected from the group consisting ofN-glycans and O-glycans; a plant oligosaccharide, preferably selected from the group consisting of N-glycans and O-glycans; fucosylated oligosaccharide, preferably selected from the group comprising 2′-fucosyllactose (2′FL), 3-fucosyllactose (3FL), 4-fucosyllactose (4FL), 6-fucosyllactose (6FL), 2′,3-difucosyllactose (diFL), lacto-N-fucopentaose I, lacto-N-neofucopentaose I, lacto-N-fucopentaose II, lacto-N-fucopentaose III, lacto-N-fucopentaose V, lacto-N-fucopentaose VI, lacto-N-neofucopentaose V, lacto-N-difucohexaose I, lacto-N-difucohexaose II, difucosyl-lacto-N-hexaose and difucosyl-lacto-N-neohexaose; sialylated oligosaccharide, preferably selected from the group comprising 3′sialyllactose (3′SL), 6′sialyllactose (6′SL), sialyllacto-N-tetraose a (LSTa), sialyllacto-N-tetraose b (LSTb), sialyllacto-N-tetraose c (LSTc), sialyllacto-N-tetraose d (LSTd), disialyllacto-N-tetraose, disialyllacto-N-neotetraose, monosialyllacto-N-hexaose, disialyllacto-N-hexaose I, disialyllacto-N-hexaose II, monosialyllacto-N-neohexaose I, monosialyllacto-N-neohexaose II, disialyllacto-N-neohexaose, 3′-sialyl-3-fucosyllactose, fucodisialyllacto-N-hexaose, disialomonofucosyllacto-N-neohexaose, sialyllacto-N-fucohexaose II, disialyllacto-N-fucopentaose II and monofucosyldisialyllacto-N-tetraose; N-acetylglucosamine containing neutral (non-charged) saccharide, preferably selected from the group comprising lacto-N-biose (LNB), N-acetyllactosamine (LacNAc), lacto-N-triose II (LN3), lacto-N-tetraose (LNT), lacto-N-neotetraose (LNnT), 6′-galactosyllactose, 3′-galactosyllactose, lacto-N-hexaose, lacto-N-neohexaose, para-lacto-N-hexaose, para-lacto-N-neohexaose; N-acetylglucosamine containing saccharide; N-acetyllactosamine containing saccharide; lacto-N-biose containing saccharide; non-fucosylated neutral (non-charged) saccharide; chitosan; chitosan comprising oligosaccharide; heparosan; chondroitin sulphate; glycosaminoglycan oligosaccharide; heparin; heparan sulphate; dermatan sulphate; hyaluronan; hyaluronic acid; and keratan sulphate.
12. Cell according to any one of previous embodiments, wherein the cell is capable of producing, preferably produces, the saccharide from one or more precursor(s), preferably the precursor is lactose.
13. Cell according to embodiment 12, wherein the cell is capable of producing, preferably produces, at least one of the one or more precursor(s), preferably the cell is capable of producing, preferably produces, all of the one or more precursor(s).
14. Cell according to any one of embodiment 12 or 13, wherein the cell is genetically engineered for the production of at least one of the one or more precursor(s), preferably the cell is genetically engineered for the production of all of the one or more precursor(s).
15. Cell according to any one of embodiments 12 to 14, wherein at least one of the one or more precursor(s) is internalized in the cell via one or more membrane protein(s).
16. Cell according to any one of previous embodiments, wherein the cell comprises a catabolic pathway for selected mono-, di- or oligosaccharides that is at least partially inactivated, the mono-, di-, or oligosaccharides being involved in and/or required for the synthesis of the saccharide.
17. Cell according to any one of previous embodiments, wherein the cell is selected from the group consisting of prokaryotic cells and eukaryotic cells, preferably from the group consisting of yeast cells, bacterial cells, archaebacterial cells, algae cells, plant cells and fungal cells.
18. Cell according to any one of previous embodiments, wherein the cell is a bacterium, fungus, yeast or a plant cell,
   preferably, the bacterium belongs to a phylum chosen from the group comprising Proteobacteria, Firmicutes, Cyanobacteria, Deinococcus-Thermus and Actinobacteria; more preferably, the bacterium belongs to a family chosen from the group comprising Enterobacteriaceae, Bacillaceae, Lactobacillaceae, Corynebacteriaceae and Vibrionaceae; even more preferably, the bacterium is chosen from the list comprising an Escherichia coli strain, a Bacillus subtilis strain, a Vibrio natriegens strain; even more preferably the Escherichia coli strain is a K-12 strain, most preferably the Escherichia coli K-12 strain is E. coli MG1655,
   preferably, the fungus belongs to a genus chosen from the group comprising Rhizopus, Dictyostelium, Penicillium, Mucor or Aspergillus,
   preferably, the yeast belongs to a genus chosen from the group comprising Saccharomyces, Zygosaccharomyces, Pichia, Komagataella, Hansenula, Yarrowia, Starmerella, Kluyveromyces, Debaromyces, Candida, Schizosaccharomyces, Schwanniomyces or Torulaspora; more preferably, the yeast is selected from the group consisting of: Saccharomyces cerevisiae, Hansenula polymorpha, Kluyveromyces lactis, Kluyveromyces marxianus, Pichia pastoris, Pichia methanolica, Pichia stipites, Candida boidinii, Schizosaccharomyces pombe, Schwanniomyces occidentalis, Torulaspora delbrueckii, Yarrowia lipolytica, Zygosaccharomyces rouxii, and Zygosaccharomyces bailii,
   preferably the plant cell is an algal cell or is derived from rose, tobacco, alfalfa, rice, tomato, cotton, rapeseed, soy, maize, or corn plant.
19. Cell according to any one of previous embodiments, wherein the cell is an E. coli or yeast with a lactose permease positive phenotype, preferably wherein the lactose permease is coded by the gene LacY or LAC12, respectively.
20. Method for the production of a saccharide, the method comprising:
   i. cultivating and/or incubating a cell of any one of previous embodiments, in cultivation and/or incubation medium under conditions permissive to produce the saccharide and the LBA,
   ii. preferably, separating the saccharide from the cultivation and/or incubation,
   iii. preferably, separating the saccharide from the LBA.
21. Method according to embodiment 20, wherein the cultivation or incubation medium comprises one or more precursor(s) that is/are used for production of the saccharide and/or the LBA.
22. Method according to any one of embodiment 20 or 21, wherein the cell produces 30 g/L or more of the saccharide in the whole broth and/or supernatant and/or wherein the saccharide in the whole broth and/or supernatant has a purity of at least 80% measured on the total amount of saccharide and its precursor(s) produced by the cell in the whole broth and/or supernatant, respectively.
23. Method according to any one of embodiments 20 to 22, wherein the less functional or knocked out synthesis of LBA confers unaffected and/or enhanced i) saccharide formation, ii) productivity, iii) biomass production, iv) cell growth and/or v) yield of the produced saccharide, relative to a corresponding non-modified cell.
24. Method according to any one of embodiments 20 to 23, wherein the cell produces a mixture comprising the saccharide and LBA, wherein the mixture comprises≤10 weight % LBA, preferably ≤9 weight % LBA, more preferably ≤8 weight % LBA, even more preferably ≤7 weight % LBA, even more preferably ≤6 weight % LBA, even more preferably ≤5 weight % LBA, even more preferably ≤4 weight % LBA, even more preferably ≤3 weight % LBA, even more preferably ≤2 weight % LBA, even more preferably ≤1 weight % LBA, even more preferably 0.5 weight % LBA, most preferably 0.1 weight % LBA.
25. Method according to any one of embodiments 20 to 24, wherein the cell produces a modified form of LBA, preferably wherein the modified form of LBA is a glycosylated LBA, more preferably wherein the modified form of LBA comprises fucosylated LBA and sialylated LBA.
26. Method according to any one of embodiments 20 to 23 or 25, wherein the cell produces no free LBA.
27. Method according to any one of embodiments 20 to 26, wherein the saccharide is recovered from the cultivation or incubation medium and/or the cell, more preferably the saccharide is purified.
28. Method according to any one of embodiments 20 to 27, wherein the saccharide is purified from the LBA and/or from the modified form of LBA.
29. Use of a cell according to any one of embodiments ito 19 for the production of a saccharide, wherein the saccharide is chosen from the list comprising monosaccharide, phosphorylated monosaccharide, activated monosaccharide, disaccharide, oligosaccharide, neutral (non-charged) oligosaccharide, a negatively charged, preferably sialylated, oligosaccharide, milk oligosaccharide, preferably a mammalian milk oligosaccharide (MMO), more preferably a human milk oligosaccharide (HMO); sialylated milk oligosaccharide, neutral (non-charged) milk oligosaccharide, fucosylated milk oligosaccharide, non-fucosylated neutral (non-charged) milk oligosaccharide, sialylated mammalian milk oligosaccharide, neutral (non-charged) mammalian milk oligosaccharide, fucosylated mammalian milk oligosaccharide, non-fucosylated neutral (non-charged) mammalian milk oligosaccharide, sialylated human milk oligosaccharide, neutral (non-charged) human milk oligosaccharide, fucosylated human milk oligosaccharide, non-fucosylated neutral (non-charged) human milk oligosaccharide; O-antigen; enterobacterial common antigen (ECA); the oligosaccharide repeats present in capsular polysaccharides; peptidoglycan; an amino-sugar; Lewis-type antigen oligosaccharide; an antigen of the human ABO blood group system; an animal oligosaccharide, preferably selected from the group consisting ofN-glycans and O-glycans; a plant oligosaccharide, preferably selected from the group consisting ofN-glycans and O-glycans; fucosylated oligosaccharide, preferably selected from the group comprising 2′-fucosyllactose (2′FL), 3-fucosyllactose (3FL), 4-fucosyllactose (4FL), 6-fucosyllactose (6FL), 2′,3-difucosyllactose (diFL), lacto-N-fucopentaose I, lacto-N-neofucopentaose I, lacto-N-fucopentaose II, lacto-N-fucopentaose III, lacto-N-fucopentaose V, lacto-N-fucopentaose VI, lacto-N-neofucopentaose V, lacto-N-difucohexaose I, lacto-N-difucohexaose II, difucosyl-lacto-N-hexaose and difucosyl-lacto-N-neohexaose; sialylated oligosaccharide, preferably selected from the group comprising 3′sialyllactose (3′SL), 6′sialyllactose (6′SL), sialyllacto-N-tetraose a (LSTa), sialyllacto-N-tetraose b (LSTb), sialyllacto-N-tetraose c (LSTc), sialyllacto-N-tetraose d (LSTd), disialyllacto-N-tetraose, disialyllacto-N-neotetraose, monosialyllacto-N-hexaose, disialyllacto-N-hexaose I, disialyllacto-N-hexaose II, monosialyllacto-N-neohexaose I, monosialyllacto-N-neohexaose II, disialyllacto-N-neohexaose, 3′-sialyl-3-fucosyllactose, fucodisialyllacto-N-hexaose, disialomonofucosyllacto-N-neohexaose, sialyllacto-N-fucohexaose II, disialyllacto-N-fucopentaose II and monofucosyldisialyllacto-N-tetraose; N-acetylglucosamine containing neutral (non-charged) saccharide, preferably selected from the group comprising lacto-N-biose (LNB), N-acetyllactosamine (LacNAc), lacto-N-triose II (LN3), lacto-N-tetraose (LNT), lacto-N-neotetraose (LNnT), 6′-galactosyllactose, 3′-galactosyllactose, lacto-N-hexaose, lacto-N-neohexaose, para-lacto-N-hexaose, para-lacto-N-neohexaose; N-acetylglucosamine containing saccharide; N-acetyllactosamine containing saccharide; lacto-N-biose containing saccharide; non-fucosylated neutral (non-charged) saccharide; chitosan; chitosan comprising oligosaccharide; heparosan; chondroitin sulphate; glycosaminoglycan oligosaccharide; heparin; heparan sulphate; dermatan sulphate; hyaluronan; hyaluronic acid; and keratan sulphate.
30. Use of a method according to any one of embodiments 20 to 28 for the production of a saccharide, wherein the saccharide is chosen from the list comprising monosaccharide, phosphorylated monosaccharide, activated monosaccharide, disaccharide, oligosaccharide, neutral (non-charged) oligosaccharide, a negatively charged, preferably sialylated, oligosaccharide, milk oligosaccharide, preferably a mammalian milk oligosaccharide (MMO), more preferably a human milk oligosaccharide (HMO); sialylated milk oligosaccharide, neutral (non-charged) milk oligosaccharide, fucosylated milk oligosaccharide, non-fucosylated neutral (non-charged) milk oligosaccharide, sialylated mammalian milk oligosaccharide, neutral (non-charged) mammalian milk oligosaccharide, fucosylated mammalian milk oligosaccharide, non-fucosylated neutral (non-charged) mammalian milk oligosaccharide, sialylated human milk oligosaccharide, neutral (non-charged) human milk oligosaccharide, fucosylated human milk oligosaccharide, non-fucosylated neutral (non-charged) human milk oligosaccharide; O-antigen; enterobacterial common antigen (ECA); the oligosaccharide repeats present in capsular polysaccharides; peptidoglycan; an amino-sugar; Lewis-type antigen oligosaccharide; an antigen of the human ABO blood group system; an animal oligosaccharide, preferably selected from the group consisting of N-glycans and O-glycans; a plant oligosaccharide, preferably selected from the group consisting of N-glycans and O-glycans; fucosylated oligosaccharide, preferably selected from the group comprising 2′-fucosyllactose (2′FL), 3-fucosyllactose (3FL), 4-fucosyllactose (4FL), 6-fucosyllactose (6FL), 2′,3-difucosyllactose (diFL), lacto-N-fucopentaose I, lacto-N-neofucopentaose I, lacto-N-fucopentaose II, lacto-N-fucopentaose III, lacto-N-fucopentaose V, lacto-N-fucopentaose VI, lacto-N-neofucopentaose V, lacto-N-difucohexaose I, lacto-N-difucohexaose II, difucosyl-lacto-N-hexaose and difucosyl-lacto-N-neohexaose; sialylated oligosaccharide, preferably selected from the group comprising 3′sialyllactose (3′SL), 6′sialyllactose (6′SL), sialyllacto-N-tetraose a (LSTa), sialyllacto-N-tetraose b (LSTb), sialyllacto-N-tetraose c (LSTc), sialyllacto-N-tetraose d (LSTd), disialyllacto-N-tetraose, disialyllacto-N-neotetraose, monosialyllacto-N-hexaose, disialyllacto-N-hexaose I, disialyllacto-N-hexaose II, monosialyllacto-N-neohexaose I, monosialyllacto-N-neohexaose II, disialyllacto-N-neohexaose, 3′-sialyl-3-fucosyllactose, fucodisialyllacto-N-hexaose, disialomonofucosyllacto-N-neohexaose, sialyllacto-N-fucohexaose II, disialyllacto-N-fucopentaose II and monofucosyldisialyllacto-N-tetraose; N-acetylglucosamine containing neutral (non-charged) saccharide, preferably selected from the group comprising lacto-N-biose (LNB), N-acetyllactosamine (LacNAc), lacto-N-triose II (LN3), lacto-N-tetraose (LNT), lacto-N-neotetraose (LNnT), 6′-galactosyllactose, 3′-galactosyllactose, lacto-N-hexaose, lacto-N-neohexaose, para-lacto-N-hexaose, para-lacto-N-neohexaose; N-acetylglucosamine containing saccharide; N-acetyllactosamine containing saccharide; lacto-N-biose containing saccharide; non-fucosylated neutral (non-charged) saccharide; chitosan; chitosan comprising oligosaccharide; heparosan; chondroitin sulphate; glycosaminoglycan oligosaccharide; heparin; heparan sulphate; dermatan sulphate; hyaluronan; hyaluronic acid; and keratan sulphate.

More specifically, the disclosure relates to the following preferred specific embodiments:
1. A cell capable of synthesizing and/or synthesizing lactobionic acid (4-O-b-galactopyranosyl-D-gluconic acid, LBA), a modified form of LBA, a glycosylated form of LBA, fucosylated LBA and/or sialylated LBA in the presence of lactose in the cultivation or incubation medium of the cell, wherein synthesis of the LBA, modified form of LBA, glycosylated form of LBA, fucosylated LBA and/or sialylated LBA is obtained by expression of at least one gene selected from the list consisting of genes encoding carbohydrate oxidase, dehydrogenase, lactonase, oxygen-dependent FAD-linked oxidoreductase, and pyrroloquinoline quinone (PQQ) oxidoreductase, the cell genetically engineered for the production of a saccharide, the cell comprising a pathway for production of the saccharide, characterized in that synthesis of the LBA, modified form of LBA, glycosylated form of LBA, fucosylated LBA and/or sialylated LBA in the cell is rendered less functional or is knocked out by rendering less functional or knock out of the at least one gene, two or more of the genes or all of the genes.
2. Cell according to preferred embodiment 1, wherein:
   the pathway for production of the saccharide is selected from the list consisting of or consisting essentially of fucosylation pathway, sialylation pathway, galactosylation pathway, N-acetylglucosaminylation pathway, N-acetylgalactosaminylation pathway, mannosylation pathway and N-acetylmannosaminylation pathway, and/or
   the pathway for production of the saccharide is selected from the list consisting of or consisting essentially of fucosylation pathway, sialylation pathway, galactosylation pathway, N-acetylglucosaminylation pathway, N-acetylgalactosaminylation pathway, mannosylation pathway and N-acetylmannosaminylation pathway and wherein the cell is genetically engineered to comprise at least one of the pathway(s) and/or the cell comprises at least one of the pathway(s) wherein at least one of the pathway(s) has/have been genetically engineered.
3. Cell according to any one of preferred embodiment 1 or 2, wherein the cell:
   possesses, expresses and/or overexpresses one or more glycosyltransferase(s) selected from the list consisting of or consisting essentially of fucosyltransferases, sialyltransferases, galactosyltransferases, glucosyltransferases, mannosyltransferases, N-acetylglucosaminyltransferases, N-acetylgalactosaminyltransferases, N-acetylmannosaminyltransferases, xylosyltransferases, glucuronyltransferases, galacturonyltransferases, glucosaminyltransferases, N-glycolylneuraminyltransferases, rhamnosyltransferases, N-acetylrhamnosyltransferases, UDP-4-amino-4,6-dideoxy-N-acetyl-β-L-altrosamine transaminases, UDP-N-acetylglucosamine enolpyruvyl transferases and fucosaminyltransferases,
   is capable of producing and/or produces one or more nucleotide-activated sugars,
   is genetically engineered for production of one or more of nucleotide-activated sugar(s),
   comprises a pathway for the synthesis of a nucleotide-activated sugar selected from the list consisting of or consisting essentially of UDP-N-acetylglucosamine (UDP-GlcNAc), UDP-N-acetylgalactosamine (UDP-GalNAc), UDP-N-acetylmannosamine (UDP-ManNAc), UDP-glucose (UDP-Glc), UDP-galactose (UDP-Gal), GDP-mannose (GDP-Man), GDP-fucose, (GDP-Fuc), UDP-glucuronate, UDP-galacturonate, UDP-2-acetamido-2,6-dideoxy-L-arabino-4-hexulose, UDP-2-acetamido-2,6-dideoxy-L-lyxo-4-hexulose, UDP-N-acetyl-L-rhamnosamine (UDP-L-RhaNAc or UDP-2-acetamido-2,6-dideoxy-L-mannose), dTDP-N-acetylfucosamine, UDP-N-acetylfucosamine (UDP-L-FucNAc or UDP-2-acetamido-2,6-dideoxy-L-galactose), UDP-N-acetyl-L-pneumosamine (UDP-L-PneNAC orUDP-2-acetamido-2,6-dideoxy-L-talose), UDP-N-acetylmuramic acid, UDP-N-acetyl-L-quinovosamine (UDP-L-QuiNAc or UDP-2-acetamido-2,6-dideoxy-L-glucose), CMP-sialic acid (CMP-Neu5Ac), CMP-Neu4Ac, CMP-Neu5Ac9N3, CMP-Neu4,5Ac2, CMP-Neu5,7Ac2, CMP-Neu5,9Ac2, CMP-Neu5,7(8,9)Ac2, CMP-N-glycolylneuraminic acid (CMP-Neu5Gc), GDP-rhamnose and UDP-xylose,
   possesses, expresses and/or overexpresses one or more genes selected from the list consisting of or consisting essentially of mannose-6-phosphate isomerase, phosphomannomutase, mannose-1-phosphate guanylyltransferase, GDP-mannose 4,6-dehydratase, GDP-L-fucose synthase, fucose permease, fucose kinase, fucose-1-phosphate guanylyltransferase, L-glutamine-D-fructose-6-phosphate aminotransferase, phosphoglucosamine mutase, N-acetylglucosamine-6-P deacetylase, N-acylglucosamine 2-epimerase, UDP-N-acetylglucosamine 2-epimerase, N-acetylmannosamine-6-phosphate 2-epimerase, UDP-GlcNAc 2-epimerase/kinase, glucosamine 6-phosphate N-acetyltransferase, N-acetylglucosamine-6-phosphate phosphatase, phosphoacetylglucosamine mutase, N-acetylglucosamine 1-phosphate uridylyltransferase, glucosamine-1-phosphate acetyltransferase, Neu5Ac synthase, N-acetylneuraminate lyase, N-acylneuraminate-9-phosphate synthase, N-acylneuraminate-9-phosphatase, sialic acid transporter, CMP kinase, CMP-sialic acid synthase, galactose-1-epimerase, galactokinase, glucokinase, galactose-1-phosphate uridylyltransferase, UDP-glucose 4-epimerase, glucose-1-phosphate uridylyltransferase, phosphoglucomutase, UDP-N-acetylglucosamine 4-epimerase, N-acetylgalactosamine kinase and UDP-N-acetylgalactosamine pyrophosphorylase, and/or
   comprises a catabolic pathway for selected mono-, di- or oligosaccharides that is at least partially inactivated, the mono-, di-, or oligosaccharides being involved in and/or required for the synthesis of the saccharide.
4. Cell according to any one of previous preferred embodiments, wherein the:
   dehydrogenase is selected from the list consisting of or consisting essentially of lactose dehydrogenase, quinoprotein glucose dehydrogenase, aldose sugar dehydrogenase, glucose/fructose dehydrogenase, glucose/sorbosone dehydrogenase, cellobiose dehydrogenase, pyrroloquinoline quinone (PQQ) dehydrogenase, and malate dehydrogenase, and/or
   carbohydrate oxidase is a lactose oxidase.
5. Cell according to any one of previous preferred embodiments, wherein the at least one gene encodes an enzyme, wherein the enzyme:
   is selected from an enzyme class selected from the list consisting of or consisting essentially of EC:1.1.3.-, EC:1.1.3.5, EC:1.1.3.4, EC:1.1.5.-, EC:1.1.5.2, and EC:1.1.99.18,
   comprises a polypeptide sequence comprising an IPR domain selected from the list consisting of or consisting essentially of IPR000172, IPR001479, IPR002372, IPR006094, IPR007867, IPR011041, IPR011042, IPR011047, 1PR012132, IPR012938, IPR012951, IPR013154, IPR013428, IPR015402, IPR015920, 1PR016166, IPR016169, IPR017511, IPR017512, IPR018391, IPR027424, IPR029056, 1PR031640, IPR036188 and IPR036318 as defined by InterPro 90.0 as released on 4 Aug. 2022,
   comprises a polypeptide sequence comprising a Panther domain selected from the list consisting of or consisting essentially of PTHR11552, PTHR13460, PTHR32303:SF4, PTHR42973 and PTHR47190 as defined by InterPro 90.0 as released on 4 Aug. 2022,
   comprises a polypeptide sequence comprising a PFAM domain selected from the list consisting of or consisting essentially of PF00732, PF01011, PF05199, PF07995, PF08031, PF08240, PF13360, PF13570, PF01565, PF16010 and PF16912 as defined by InterPro 90.0 as released on 4 Aug. 2022,
   comprises a polypeptide sequence comprising the conserved protein domain cd10280 as defined by InterPro 90.0 as released on 4 Aug. 2022,
   is part of a NOG family selected from the list consisting of or consisting essentially of COG0277, COG2133, COG2303 and COG4993 as defined by eggNOG5.0 as released in 2019 and/or
   uses a cofactor chosen from the list consisting of or consisting essentially of pyrroloquinoline quinone (PQQ), FAD, a divalent metal cation, Ca²⁺, Cu²⁺, Mg²⁺, Zn²⁺, heme, heme C and pyridoxal 5′-phosphate.
6. Cell according to any one of previous preferred embodiments, wherein at least one gene involved in the synthesis and/or import of:
   a co-factor that is involved in the synthesis of LBA, a modified form of LBA, a glycosylated form of LBA, fucosylated LBA and/or sialylated LBA is rendered less functional or knocked out, and/or
   a co-factor that is selected from the list consisting of or consisting essentially of pyrroloquinoline quinone (PQQ), FAD, a divalent metal cation, Ca²⁺, Cu²⁺, Mg²⁺, Zn²⁺, heme, heme C and pyridoxal 5′-phosphate and that is involved in the synthesis of LBA, a modified form of LBA, a glycosylated form of LBA, fucosylated LBA and/or sialylated LBA is rendered less functional or knocked out.
7. Cell according to any one of previous preferred embodiments, wherein the at least one gene is rendered less functional by insertion, deletion and/or modification of one or more nucleotide(s) in one or more polynucleotide sequence(s) selected from the list consisting of or consisting essentially of promoter sequence, ribosome binding site, untranslated region, coding sequence and transcription terminator sequence of the at least one gene.
8. Cell according to any one of previous preferred embodiments, wherein the cell comprises a quinoprotein glucose dehydrogenase gene that is rendered less functional by insertion, deletion and/or modification of one or more nucleotide(s) in one or more polynucleotide sequence(s) selected from the list consisting of or consisting essentially of promoter sequence, ribosome binding site, untranslated region, coding sequence and transcription terminator sequence of the quinoprotein glucose dehydrogenase gene.
9. Cell according to preferred embodiment 8, wherein the cell further comprises an aldose sugar dehydrogenase gene that is rendered less functional by insertion, deletion and/or modification of one or more nucleotide(s) in one or more polynucleotide sequence(s) selected from the list consisting of or consisting essentially of promoter sequence, ribosome binding site, untranslated region, coding sequence and transcription terminator sequence of the aldose sugar dehydrogenase gene.
10. Cell according to any one of preferred embodiments ito 7, wherein the cell comprises an aldose sugar dehydrogenase gene that is rendered less functional by insertion, deletion and/or modification of one or more nucleotide(s) in one or more polynucleotide sequence(s) selected from the list consisting of or consisting essentially of promoter sequence, ribosome binding site, untranslated region, coding sequence and transcription terminator sequence of the aldose sugar dehydrogenase gene.
11. Cell according to preferred embodiment 10, wherein the cell further comprises a quinoprotein glucose dehydrogenase gene that is rendered less functional by insertion, deletion and/or modification of one or more nucleotide(s) in one or more polynucleotide sequence(s) selected from the list consisting of or consisting essentially of promoter sequence, ribosome binding site, untranslated region, coding sequence and transcription terminator sequence of the quinoprotein glucose dehydrogenase gene.
12. Cell according to any one of previous preferred embodiments, wherein the saccharide is selected from the list consisting of or consisting essentially of monosaccharide; phosphorylated monosaccharide; activated monosaccharide; disaccharide; oligosaccharide; neutral (non-charged) oligosaccharide; negatively charged oligosaccharide; sialylated oligosaccharide; milk oligosaccharide; mammalian milk oligosaccharide (MMO); human milk oligosaccharide (HMO); sialylated milk oligosaccharide; neutral (non-charged) milk oligosaccharide; fucosylated milk oligosaccharide; non-fucosylated neutral (non-charged) milk oligosaccharide; sialylated mammalian milk oligosaccharide; neutral (non-charged) mammalian milk oligosaccharide; fucosylated mammalian milk oligosaccharide; non-fucosylated neutral (non-charged) mammalian milk oligosaccharide; sialylated human milk oligosaccharide; neutral (non-charged) human milk oligosaccharide; fucosylated human milk oligosaccharide; non-fucosylated neutral (non-charged) human milk oligosaccharide; O-antigen; enterobacterial common antigen (ECA); the oligosaccharide repeats present in capsular polysaccharides; peptidoglycan; an amino-sugar; Lewis-type antigen oligosaccharide; an antigen of the human ABO blood group system; an animal oligosaccharide; animal oligosaccharide selected from the list consisting of N-glycans and O-glycans; a plant oligosaccharide; plant oligosaccharide selected from the list consisting of N-glycans and O-glycans; fucosylated oligosaccharide; fucosylated oligosaccharide selected from the list comprising 2′-fucosyllactose (2′FL), 3-fucosyllactose (3FL), 4-fucosyllactose (4FL), 6-fucosyllactose (6FL), 2′,3-difucosyllactose (diFL), lacto-N-fucopentaose I, lacto-N-neofucopentaose I, lacto-N-fucopentaose II, lacto-N-fucopentaose III, lacto-N-fucopentaose V, lacto-N-fucopentaose VI, lacto-N-neofucopentaose V, lacto-N-difucohexaose I, lacto-N-difucohexaose II, difucosyl-lacto-N-hexaose and difucosyl-lacto-N-neohexaose; sialylated oligosaccharide selected from the list comprising 3′sialyllactose (3′SL), 6′sialyllactose (6′SL), sialyllacto-N-tetraose a (LSTa), sialyllacto-N-tetraose b (LSTb), sialyllacto-N-tetraose c (LSTc), sialyllacto-N-tetraose d (LSTd), disialyllacto-N-tetraose, disialyllacto-N-neotetraose, monosialyllacto-N-hexaose, disialyllacto-N-hexaose I, disialyllacto-N-hexaose II, monosialyllacto-N-neohexaose I, monosialyllacto-N-neohexaose II, disialyllacto-N-neohexaose, 3′-sialyl-3-fucosyllactose, fucodisialyllacto-N-hexaose, disialomonofucosyllacto-N-neohexaose, sialyllacto-N-fucohexaose II, disialyllacto-N-fucopentaose II and monofucosyldisialyllacto-N-tetraose; N-acetylglucosamine containing neutral (non-charged) saccharide; N-acetylglucosamine containing neutral (non-charged) saccharide selected from the list comprising lacto-N-biose (LNB), N-acetyllactosamine (LacNAc), lacto-N-triose II (LN3), lacto-N-tetraose (LNT), lacto-N-neotetraose (LNnT), 6′-galactosyllactose, 3′-galactosyllactose, lacto-N-hexaose, lacto-N-neohexaose, para-lacto-N-hexaose, para-lacto-N-neohexaose; N-acetylglucosamine containing saccharide; N-acetyllactosamine containing saccharide; lacto-N-biose containing saccharide; non-fucosylated neutral (non-charged) saccharide; chitosan; chitosan comprising oligosaccharide; heparosan; chondroitin sulphate; glycosaminoglycan oligosaccharide; heparin; heparan sulphate; dermatan sulphate; hyaluronan; hyaluronic acid; and keratan sulphate.
13. Cell according to any one of previous preferred embodiments, wherein the cell:
   is capable of producing and/or produces the saccharide, LBA, modified form of LBA, glycosylated form of LBA, fucosylated LBA and/or sialylated LBA from one or more precursor(s),
   is capable of producing and/or produces the saccharide, LBA, modified form of LBA, glycosylated form of LBA, fucosylated LBA and/or sialylated LBA from lactose,
   is capable of producing and/or produces at least one precursor that is used to produce the saccharide, LBA, modified form of LBA, glycosylated form of LBA, fucosylated LBA and/or sialylated LBA,
   is capable of producing and/or produces all precursors that are used to produce the saccharide, LBA, modified form of LBA, glycosylated form of LBA, fucosylated LBA and/or sialylated LBA,
   is genetically engineered for the production of at least one precursor that is used to produce the saccharide, LBA, modified form of LBA, glycosylated form of LBA, fucosylated LBA and/or sialylated LBA, and/or
   is genetically engineered for the production of all precursors that are used to produce the saccharide, LBA, modified form of LBA, glycosylated form of LBA, fucosylated LBA and/or sialylated LBA.
14. Cell according to preferred embodiment 13, wherein at least one of the one or more precursor(s) is internalized in the cell via one or more membrane protein(s).
15. Cell according to any one of previous preferred embodiments, wherein the cell:
   is selected from the group consisting of prokaryotic cells and eukaryotic cells,
   is selected from the group consisting of yeast cells, bacterial cells, archaebacterial cells, algae cells, plant cells and fungal cells,
   an E. coli or yeast with a lactose permease positive phenotype, and/or
   an E. coli or yeast with a lactose permease positive phenotype wherein the lactose permease is coded by the gene LacY or LAC12, respectively.
16. Method for the production of a saccharide, the method comprising:
   i. cultivating and/or incubating a cell of any one of previous preferred embodiments, in cultivation and/or incubation medium under conditions permissive to produce the saccharide and any one or more of lactobionic acid (LBA), a modified form of LBA, a glycosylated form of LBA, fucosylated LBA and/or sialylated LBA, and
   ii. (1) separating the saccharide from the cultivation and/or incubation, and/or
      (2) separating the saccharide from the LBA, modified form of LBA, glycosylated form of LBA, fucosylated LBA and/or sialylated LBA.
17. Method according to preferred embodiment 16, wherein the cultivation or incubation medium comprises one or more precursor(s) that is/are used for production of the saccharide, LBA, modified form of LBA, glycosylated form of LBA, fucosylated LBA and/or sialylated LBA.
18. Method according to any one of preferred embodiment 16 or 17, wherein the cell produces 30 g/L or more of the saccharide in the whole broth and/or supernatant and/or wherein the saccharide in the whole broth and/or supernatant has a purity of at least 80% measured on the total amount of saccharide and its precursor(s) produced by the cell in the whole broth and/or supernatant, respectively.
19. Method according to any one of preferred embodiments 16 to 18, wherein the less functional or knocked out synthesis of LBA, modified form of LBA, glycosylated form of LBA, fucosylated LBA and/or sialylated LBA confers unaffected and/or enhanced i) saccharide formation, ii) productivity, iii) biomass production, iv) cell growth and/or v) yield of the produced saccharide, relative to a corresponding non-modified cell.
20. Method according to any one of preferred embodiments 16 to 19, wherein the cell produces a mixture comprising the saccharide and any one or more of the LBA, modified form of LBA, glycosylated form of LBA, fucosylated LBA and/or sialylated LBA wherein the mixture comprises≤10 weight % of the LBA, modified form of LBA, glycosylated form of LBA, fucosylated LBA and/or sialylated LBA, ≤9 weight % of the LBA, modified form of LBA, glycosylated form of LBA, fucosylated LBA and/or sialylated LBA, ≤8 weight % of the LBA, modified form of LBA, glycosylated form of LBA, fucosylated LBA and/or sialylated LBA, ≤7 weight % of the LBA, modified form of LBA, glycosylated form of LBA, fucosylated LBA and/or sialylated LBA, ≤6 weight % of the LBA, modified form of LBA, glycosylated form of LBA, fucosylated LBA and/or sialylated LBA, ≤5 weight % of the LBA, modified form of LBA, glycosylated form of LBA, fucosylated LBA and/or sialylated LBA, ≤4 weight % of the LBA, modified form of LBA, glycosylated form of LBA, fucosylated LBA and/or sialylated LBA, ≤3 weight % of the LBA, modified form of LBA, glycosylated form of LBA, fucosylated LBA and/or sialylated LBA, ≤2 weight % of the LBA, modified form of LBA, glycosylated form of LBA, fucosylated LBA and/or sialylated LBA, ≤1 weight % of the LBA, modified form of LBA, glycosylated form of LBA, fucosylated LBA and/or sialylated LBA, ≤0.5 weight % of the LBA, modified form of LBA, glycosylated form of LBA, fucosylated LBA and/or sialylated LBA and/or ≤0.1 weight % of the LBA, modified form of LBA, glycosylated form of LBA, fucosylated LBA and/or sialylated LBA.
21. Method according to any one of preferred embodiments 16 to 19, wherein the cell produces no LBA.
22. Method according to any one of preferred embodiments 16 to 21, wherein the saccharide is recovered from the cultivation or incubation medium and/or the cell, and/or wherein the saccharide is purified.
23. Method according to any one of preferred embodiments 16 to 22, wherein the saccharide is purified from (i) the LBA and/or from (ii) the modified form of LBA, glycosylated form of LBA, fucosylated LBA and/or sialylated LBA.
24. Use of a cell according to any one of preferred embodiments 1 to 15 for the production of a saccharide, wherein the saccharide is selected from the list consisting of or consisting essentially of monosaccharide; phosphorylated monosaccharide; activated monosaccharide; disaccharide; oligosaccharide; neutral (non-charged) oligosaccharide; negatively charged oligosaccharide; sialylated oligosaccharide; milk oligosaccharide; mammalian milk oligosaccharide (MMO); human milk oligosaccharide (HMO); sialylated milk oligosaccharide; neutral (non-charged) milk oligosaccharide; fucosylated milk oligosaccharide; non-fucosylated neutral (non-charged) milk oligosaccharide; sialylated mammalian milk oligosaccharide; neutral (non-charged) mammalian milk oligosaccharide; fucosylated mammalian milk oligosaccharide; non-fucosylated neutral (non-charged) mammalian milk oligosaccharide; sialylated human milk oligosaccharide; neutral (non-charged) human milk oligosaccharide; fucosylated human milk oligosaccharide; non-fucosylated neutral (non-charged) human milk oligosaccharide; O-antigen; enterobacterial common antigen (ECA); the oligosaccharide repeats present in capsular polysaccharides; peptidoglycan; an amino-sugar; Lewis-type antigen oligosaccharide; an antigen of the human ABO blood group system; an animal oligosaccharide; an animal oligosaccharide selected from the group consisting of N-glycans and O-glycans; a plant oligosaccharide; a plant oligosaccharide selected from the group consisting of N-glycans and O-glycans; fucosylated oligosaccharide; fucosylated oligosaccharide selected from the group comprising 2′-fucosyllactose (2′FL), 3-fucosyllactose (3FL), 4-fucosyllactose (4FL), 6-fucosyllactose (6FL), 2′,3-difucosyllactose (diFL), lacto-N-fucopentaose I, lacto-N-neofucopentaose I, lacto-N-fucopentaose II, lacto-N-fucopentaose III, lacto-N-fucopentaose V, lacto-N-fucopentaose VI, lacto-N-neofucopentaose V, lacto-N-difucohexaose I, lacto-N-difucohexaose II, difucosyl-lacto-N-hexaose and difucosyl-lacto-N-neohexaose; sialylated oligosaccharide selected from the group comprising 3′sialyllactose (3′SL), 6′sialyllactose (6′SL), sialyllacto-N-tetraose a (LSTa), sialyllacto-N-tetraose b (LSTb), sialyllacto-N-tetraose c (LSTc), sialyllacto-N-tetraose d (LSTd), disialyllacto-N-tetraose, disialyllacto-N-neotetraose, monosialyllacto-N-hexaose, disialyllacto-N-hexaose I, disialyllacto-N-hexaose II, monosialyllacto-N-neohexaose I, monosialyllacto-N-neohexaose II, disialyllacto-N-neohexaose, 3′-sialyl-3-fucosyllactose, fucodisialyllacto-N-hexaose, disialomonofucosyllacto-N-neohexaose, sialyllacto-N-fucohexaose II, disialyllacto-N-fucopentaose II and monofucosyldisialyllacto-N-tetraose; N-acetylglucosamine containing neutral (non-charged) saccharide; N-acetylglucosamine containing neutral (non-charged) saccharide selected from the group comprising lacto-N-biose (LNB), N-acetyllactosamine (LacNAc), lacto-N-triose II (LN3), lacto-N-tetraose (LNT), lacto-N-neotetraose (LNnT), 6′-galactosyllactose, 3′-galactosyllactose, lacto-N-hexaose, lacto-N-neohexaose, para-lacto-N-hexaose, para-lacto-N-neohexaose; N-acetylglucosamine containing saccharide; N-acetyllactosamine containing saccharide; lacto-N-biose containing saccharide; non-fucosylated neutral (non-charged) saccharide; chitosan; chitosan comprising oligosaccharide; heparosan; chondroitin sulphate; glycosaminoglycan oligosaccharide; heparin; heparan sulphate; dermatan sulphate; hyaluronan; hyaluronic acid; and keratan sulphate.
25. Use of a method according to any one of preferred embodiments 16 to 23 for the production of a saccharide, wherein the saccharide is selected from the list consisting of or consisting essentially of monosaccharide; phosphorylated monosaccharide; activated monosaccharide; disaccharide; oligosaccharide; neutral (non-charged) oligosaccharide; negatively charged oligosaccharide; sialylated oligosaccharide; milk oligosaccharide; mammalian milk oligosaccharide (MMO); human milk oligosaccharide (HMO); sialylated milk oligosaccharide; neutral (non-charged) milk oligosaccharide; fucosylated milk oligosaccharide; non-fucosylated neutral (non-charged) milk oligosaccharide; sialylated mammalian milk oligosaccharide; neutral (non-charged) mammalian milk oligosaccharide; fucosylated mammalian milk oligosaccharide; non-fucosylated neutral (non-charged) mammalian milk oligosaccharide; sialylated human milk oligosaccharide; neutral (non-charged) human milk oligosaccharide; fucosylated human milk oligosaccharide; non-fucosylated neutral (non-charged) human milk oligosaccharide; O-antigen; enterobacterial common antigen (ECA); the oligosaccharide repeats present in capsular polysaccharides; peptidoglycan; an amino-sugar; Lewis-type antigen oligosaccharide; an antigen of the human ABO blood group system; an animal oligosaccharide; an animal oligosaccharide selected from the group consisting of N-glycans and O-glycans; a plant oligosaccharide; a plant oligosaccharide selected from the group consisting of N-glycans and O-glycans; fucosylated oligosaccharide; fucosylated oligosaccharide selected from the group comprising 2′-fucosyllactose (2′FL), 3-fucosyllactose (3FL), 4-fucosyllactose (4FL), 6-fucosyllactose (6FL), 2′,3-difucosyllactose (diFL), lacto-N-fucopentaose I, lacto-N-neofucopentaose I, lacto-N-fucopentaose II, lacto-N-fucopentaose III, lacto-N-fucopentaose V, lacto-N-fucopentaose VI, lacto-N-neofucopentaose V, lacto-N-difucohexaose I, lacto-N-difucohexaose II, difucosyl-lacto-N-hexaose and difucosyl-lacto-N-neohexaose; sialylated oligosaccharide selected from the group comprising 3′sialyllactose (3′SL), 6′sialyllactose (6′SL), sialyllacto-N-tetraose a (LSTa), sialyllacto-N-tetraose b (LSTb), sialyllacto-N-tetraose c (LSTc), sialyllacto-N-tetraose d (LSTd), disialyllacto-N-tetraose, disialyllacto-N-neotetraose, monosialyllacto-N-hexaose, disialyllacto-N-hexaose I, disialyllacto-N-hexaose II, monosialyllacto-N-neohexaose I, monosialyllacto-N-neohexaose II, disialyllacto-N-neohexaose, 3′-sialyl-3-fucosyllactose, fucodisialyllacto-N-hexaose, disialomonofucosyllacto-N-neohexaose, sialyllacto-N-fucohexaose II, disialyllacto-N-fucopentaose II and monofucosyldisialyllacto-N-tetraose; N-acetylglucosamine containing neutral (non-charged) saccharide; N-acetylglucosamine containing neutral (non-charged) saccharide selected from the group comprising lacto-N-biose (LNB), N-acetyllactosamine (LacNAc), lacto-N-triose II (LN3), lacto-N-tetraose (LNT), lacto-N-neotetraose (LNnT), 6′-galactosyllactose, 3′-galactosyllactose, lacto-N-hexaose, lacto-N-neohexaose, para-lacto-N-hexaose, para-lacto-N-neohexaose; N-acetylglucosamine containing saccharide; N-acetyllactosamine containing saccharide; lacto-N-biose containing saccharide; non-fucosylated neutral (non-charged) saccharide; chitosan; chitosan comprising oligosaccharide; heparosan; chondroitin sulphate; glycosaminoglycan oligosaccharide; heparin; heparan sulphate; dermatan sulphate; hyaluronan; hyaluronic acid; and keratan sulphate.
26. A mixture comprising, consisting of or consisting essentially of (i) a saccharide and (ii) LBA, a modified form of LBA, a glycosylated form of LBA, fucosylated LBA and/or sialylated LBA wherein the saccharide, LBA, modified form of LBA, glycosylated form of LBA, fucosylated LBA and/or sialylated LBA are obtainable or obtained by a method according to any one of preferred embodiments 16 to 23.
27. A mixture comprising, consisting of or consisting essentially of (i) a saccharide and (ii) ≤10 weight % LBA, ≤9 weight % LBA, ≤8 weight % LBA, ≤7 weight % LBA, 6 weight % LBA, 5 weight % LBA, 4 weight % LBA, 3 weight % LBA, 2 weight % LBA, ≤1 weight % LBA, 0.5 weight % LBA and/or ≤0.1 weight % LBA, wherein the saccharide and LBA are obtainable or obtained by a method according to any one of preferred embodiments 16 to 20, 22 or 23.
28. A mixture comprising, consisting of or consisting essentially of a saccharide and (i)≤10 weight % LBA, ≤9 weight % LBA, ≤8 weight % LBA, ≤7 weight % LBA, ≤6 weight % LBA, ≤5 weight % LBA, ≤4 weight % LBA, ≤3 weight % LBA, ≤2 weight % LBA, ≤1 weight % LBA, ≤0.5 weight % LBA and/or ≤0.1 weight % LBA and/or (ii)≤10 weight % of a modified form of LBA, a glycosylated form of LBA, fucosylated LBA and/or sialylated LBA; ≤9 weight % of a modified form of LBA, a glycosylated form of LBA, fucosylated LBA and/or sialylated LBA; ≤8 weight % of a modified form of LBA, a glycosylated form of LBA, fucosylated LBA and/or sialylated LBA; ≤7 weight % of a modified form of LBA, a glycosylated form of LBA, fucosylated LBA and/or sialylated LBA; ≤6 weight % of a modified form of LBA, a glycosylated form of LBA, fucosylated LBA and/or sialylated LBA; ≤5 weight % of a modified form of LBA, a glycosylated form of LBA, fucosylated LBA and/or sialylated LBA; ≤4 weight % of a modified form of LBA, a glycosylated form of LBA, fucosylated LBA and/or sialylated LBA; ≤2 weight % of a modified form of LBA, a glycosylated form of LBA, fucosylated LBA and/or sialylated LBA; ≤1 weight % of a modified form of LBA, a glycosylated form of LBA, fucosylated LBA and/or sialylated LBA; ≤0.5 weight % of a modified form of LBA, a glycosylated form of LBA, fucosylated LBA and/or sialylated LBA and/or ≤0.1 weight % of a modified form of LBA, a glycosylated form of LBA, fucosylated LBA and/or sialylated LBA, wherein the saccharide, LBA, modified form of LBA, glycosylated form of LBA, fucosylated LBA and/or sialylated LBA are obtainable or obtained by a method according to any one of preferred embodiments 16 to 23.

The disclosure will be described in more detail in the examples. The following drawing and examples will serve as further illustration and clarification of the disclosure and are not intended to be limiting.

### EXAMPLES

### Example 1. Materials and Methods

A. Escherichia coli

### Media and Cultivation

The Luria Broth (LB) medium consisted of 1% tryptone peptone (Difco, Erembodegem, Belgium), 0.5% yeast extract (Difco) and 0.5% sodium chloride (VWR. Leuven, Belgium). The minimal medium used in cultivation experiments in 96-well plates or in shake flasks contained 2.00 g/L NH₄Cl, 5.00 g/L (NH₄)₂SO₄, 2.993 g/L KH₂PO₄, 7.315 g/L K₂HPO₄, 8.372 g/L MOPS, 0.5 g/L NaCl, 0.5 g/L MgSO₄·7H₂O, 30 g/L sucrose or 30 g/L glycerol, 1 ml/L vitamin solution, 100 μl/L molybdate solution, and 1 mL/L selenium solution. As precursor(s) and/or acceptor(s) for saccharide synthesis, compounds like, e.g., galactose, glucose, fructose, fucose, lactose, sialic acid, N-acetyllactosamine (LacNAc), lacto-N-biose (LNB) could be added to the medium. For LBA production, a cofactor like, e.g., pyrroloquinoline quinone (PQQ), FAD, a divalent metal cation, Ca²⁺, Cu²⁺, Mg²⁺, Zn²⁺, heme, heme C and pyridoxal 5′-phosphate may be added. The minimal medium was set to a pH of 7 with 1M KOH. Vitamin solution consisted of 3.6 g/L FeCl_{2.4}H₂O, 5.0 g/L CaCl₂·2H₂O, 1.3 g/L MnCl₂·2H₂O, 0.38 g/L CuCl₂·2H₂O, 0.5 g/L CoCl₂·6H₂O, 0.94 g/L ZnCl₂, 0.0311 g/L H₃BO₄, 0.4 g/L Na₂EDTA·2H₂O and 1.01 g/L thiamine·HCl. The molybdate solution contained 0.967 g/L NaMoO₄·2H₂O. The selenium solution contained 42 g/L Seo2. The minimal medium for fermentations contained 6.75 g/L NH₄Cl, 1.25 g/L (NH₄)₂SO₄, 2.93 g/L KH₂PO₄ and 7.31 g/L KH₂PO₄, 0.5 g/L NaCl, 0.5 g/L MgSO₄·7H₂O, 30 g/L sucrose or 30 g/L glycerol, 1 mL/L vitamin solution, 100 μL/L molybdate solution, and 1 mL/L selenium solution with the same composition as described above. As specified in the respective examples, 20 g/L lactose, was additionally added to the medium. Complex medium was sterilized by autoclaving (121° C., 21 minutes) and minimal medium by filtration (0.22 μm Sartorius). When necessary, the medium was made selective by adding an antibiotic: e.g., chloramphenicol (20 mg/L), carbenicillin (100 mg/L), spectinomycin (40 mg/L) and/or kanamycin (50 mg/L). A preculture of 96-well microtiter plate experiments was started from a cryovial, in 150 μL LB and was incubated overnight at 37° C. on an orbital shaker at 800 rpm. This culture was used as inoculum for a 96-well square microtiter plate, with 400 μL minimal medium by diluting 400×. These final 96-well culture plates were then incubated at 37° C. on an orbital shaker at 800 rpm for 72 hours, or shorter, or longer. To measure sugar concentrations at the end of the cultivation experiment whole broth samples were taken from each well by boiling the culture broth for 15 minutes at 60° C. before spinning down the cells (=average of intra- and extracellular sugar concentrations). A preculture for the bioreactor was started from an entire 1 mL cryovial of a certain strain, inoculated in 250 mL or 500 mL minimal medium in a 1 L or 2.5 L shake flask and incubated for 24 hours at 37° C. on an orbital shaker at 200 rpm. A 5 L bioreactor was then inoculated (250 mL inoculum in 2 L batch medium); the process was controlled by MFCS control software (Sartorius Stedim Biotech, Melsungen, Germany). Culturing conditions were set to 37° C., and maximal stirring; pressure gas flow rates were dependent on the strain and bioreactor. The pH was controlled at 6.8 using 0.5 M H₂S0₄ and 20% NH₄OH. The exhaust gas was cooled. 10% solution of silicone antifoaming agent was added when foaming raised during the fermentation.

### Strains and Mutations

Escherichia coli K12 MG1655 [λ-, F-, rph-1] was obtained from the Coli Genetic Stock Center (US), CGSC Strain #: 7740, in March 2007. Gene disruptions, gene introductions and gene replacements were performed using the technique published by Datsenko and Wanner (PNAS 97 (2000), 6640-6645). All constitutive promoters, UTRs and terminator sequences originated from the libraries described by Cambray et al. (Nucleic Acids Res. 2013, 41(9), 5139-5148), Dunn et al. (Nucleic Acids Res. 1980, 8, 2119-2132), Edens et al. (Nucleic Acids Res. 1975, 2, 1811-1820), Kim and Lee (FEBS Letters 1997, 407, 353-356) and Mutalik et al. (Nat. Methods 2013, No. 10, 354-360). Genes were ordered synthetically at Twist Bioscience (twistbioscience.com) or IDT (eu.idtdna.com) and the codon usage was adapted using the tools of the supplier. All strains were stored in cryovials at −80° C. (overnight LB culture mixed in a 1:1 ratio with 70% glycerol).

In an example for GDP-fucose production, the mutant strain was derived from E. coli K12 MG1655 comprising a knock-out of the E. coli wcaJ gene and genomic knock-ins of constitutive transcriptional units containing a sucrose transporter like, e.g., CscB from E. coli W (UniProt ID E0IXR1), a fructose kinase like, e.g., Frk originating from Zymomonas mobilis (UniProt ID Q03417) and a sucrose phosphorylase like, e.g., BaSP originating from Bifidobacterium adolescentis (UniProt ID AOZZH6). GDP-fucose production can further be optimized in the mutant E. coli strain by genomic knock-outs of any one or more of the E. coli genes comprising glgC, agp, pfkA, pfkB, pgi, arcA, icR, pgi and Ion as described in WO2016075243 and WO2012007481. GDP-fucose production can additionally be optimized comprising genomic knock-ins of constitutive transcriptional units for a mannose-6-phosphate isomerase like, e.g., manA from E. coli (UniProt ID P00946), a phosphomannomutase like, e.g., manB from E. coli (UniProt ID P24175), a mannose-1-phosphate guanylyltransferase like, e.g., manC from E. coli (UniProt ID P24174), a GDP-mannose 4,6-dehydratase like, e.g., gmd from E. coli (UniProt ID P0AC88) and a GDP-L-fucose synthase like, e.g., fcl from E. coli (UniProt ID P32055). GDP-fucose production can also be obtained by genomic knock-outs of the E. coli fucK and fucl genes and genomic knock-ins of constitutive transcriptional units containing a fucose permease like, e.g., fucP from E. coli (UniProt ID P11551) and a bifunctional enzyme with fucose kinase/fucose-1-phosphate guanylyltransferase activity like, e.g., fkp from Bacteroides fragilis (UniProt ID SUV40286.1). All mutant strains can be additionally modified with genomic knock-outs of the E. coli LacZ, LacY and LacA genes and with a genomic knock-in of a constitutive transcriptional unit for a lactose permease like, e.g., the E. coli LacY (UniProt ID P02920). For production of a fucosylated saccharide, the mutant GDP-fucose production strain was additionally modified with an expression plasmid comprising a constitutive transcriptional unit for a fucosyltransferase, like, e.g., the α-1,2-fucosyltransferase HpFutC from H. pylori (UniProt ID Q9X435) to produce 2′-fucosyllactose (2′FL) or the α-1,3-fucosyltransferase HpFucT from H. pylori (UniProt ID 030511) to produce 3-fucosyllactose (3-FL), or both the α-1,2-fucosyltransferase HpFutC from H. pylori (UniProt ID Q9X435) and the α-1,3-fucosyltransferase HpFucT from H. pylori (UniProt ID 030511) to produce difucosyllactose (DiFL).

In an example to produce lacto-N-triose (LN3, GlcNAc-β1,3-Gal-β1,4-Glc), the mutant strain was derived from E. coli K12 MG1655 and modified with a knock-out of the E. coli lacZ, lacY, lacA and nagB genes and with genomic knock-ins of constitutive transcriptional units for a lactose permease like, e.g., the E. coli LacY (UniProt ID P02920) and a galactoside β-1,3-N-acetylglucosaminyltransferase like, e.g., lgtA (UniProt ID Q9JXQ6) from N. meningitidis. In an example for production of LN3 derived oligosaccharides like lacto-N-tetraose (LNT, Gal-β1,3-GlcNAc-β1,3-Gal-β1,4-Glc), the mutant LN3 producing strain was further modified with a constitutive transcriptional unit delivered to the strain either via genomic knock-in or from an expression plasmid for an N-acetylglucosamine β-1,3-galactosyltransferase like, e.g., wbgO (Uniprot ID D3QY14) from E. coli O55:H7. In an example for production of LN3 derived oligosaccharides like lacto-N-neotetraose (LNnT, Gal-β1,4-GlcNAc-β1,3-Gal-β1,4-Glc), the mutant LN3 producing strain was further modified with a constitutive transcriptional unit delivered to the strain either via genomic knock-in or from an expression plasmid for an N-acetylglucosamine β-1,4-galactosyltransferase like, e.g., LgtB (Uniprot ID Q51116, sequence version 02, 1 Dec. 2000) from N. meningitidis. LN3, LNT and/or LNnT production can further be optimized in the mutant E. coli strains with genomic knock-outs of the E. coli genes comprising any one or more of galT, ushA, ldhA and agp. The mutant LN3, LNT and/or LNnT producing strains can also be optionally modified for enhanced UDP-GlcNAc production with a genomic knock-in of a constitutive transcriptional unit for an L-glutamine-D-fructose-6-phosphate aminotransferase like, e.g., the E. coli glmS (UniProt ID P17169, sequence version 04, 23 Jan. 2007). The mutant E. coli strains can also optionally be adapted with a genomic knock-in of a constitutive transcriptional unit for an UDP-glucose-4-epimerase like, e.g., galE from E. coli (UniProt ID P09147), a phosphoglucosamine mutase like, e.g., glmM from E. coli (UniProt ID P31120, sequence version 03, 23 Jan. 2007) and an N-acetylglucosamine-1-phosphate uridylyltransferase/glucosamine-1-phosphate acetyltransferase like, e.g., glmU from E. coli (UniProt ID P0ACC7). The mutant LN3, LNT and/or LNnT producing E. coli strains can also optionally be adapted for growth on sucrose via genomic knock-ins of constitutive transcriptional units containing a sucrose transporter like, e.g., CscB from E. coli W (UniProt ID E0IXR1), a fructose kinase like, e.g., Frk originating from Zymomonas mobilis (UniProt ID Q03417) and a sucrose phosphorylase like, e.g., BaSP originating from Bifidobacterium adolescentis (UniProt ID A0ZZH6).

In an example for sialic acid production, the mutant strain was derived from E. coli K12 MG1655 comprising genomic knock-ins of constitutive transcriptional units containing one or more copies of a glucosamine 6-phosphate N-acetyltransferase like, e.g., GNA1 from Saccharomyces cerevisiae (UniProt ID P43577), an N-acetylglucosamine 2-epimerase like, e.g., AGE from Bacteroides ovatus (UniProt ID A7LVG6) and an N-acetylneuraminate synthase like, e.g., NeuB from N. meningitidis (UniProt ID E0NCD4).

Alternatively, and/or additionally, sialic acid production can be obtained by genomic knock-ins of constitutive transcriptional units containing an UDP-N-acetylglucosamine 2-epimerase like, e.g., NeuC from C. jejuni (UniProt ID Q93MP8) and an N-acetylneuraminate synthase like, e.g., NeuB from N. meningitidis (UniProt ID E0NCD4). Alternatively and/or additionally, sialic acid production can be obtained by genomic knock-ins of constitutive transcriptional units containing a phosphoglucosamine mutase like, e.g., glmM from E. coli (UniProt ID P31120, sequence version 03, 23 Jan. 2007), an N-acetylglucosamine-1-phosphate uridyltransferase/glucosamine-1-phosphate acetyltransferase like, e.g., glmU from E. coli (UniProt ID P0ACC7), an UDP-N-acetylglucosamine 2-epimerase like, e.g., NeuC from C. jejuni (UniProt ID Q93MP8) and an N-acetylneuraminate synthase like, e.g., NeuB from N. meningitidis (UniProt ID E0NCD4). Alternatively, and/or additionally, sialic acid production can be obtained by genomic knock-ins of constitutive transcriptional units containing a bifunctional UDP-GlcNAc 2-epimerase/N-acetylmannosamine kinase like, e.g., from Mus musculus (strain C57BL/6J) (UniProt ID Q91WG8), an N-acylneuraminate-9-phosphate synthetase like, e.g., from Pseudomonas sp. UW4 (UniProt ID K9NPH9) and an N-acylneuraminate-9-phosphatase like, e.g., from Bacteroides thetaiotaomicron (UniProt ID Q8A712).

Sialic acid production can further be optimized in the mutant E. coli strain with genomic knock-outs of the E. coli genes comprising any one or more of nagA, nagB, nagC, nagD, nagE, nanA, nanE, nanK, manX, manY and manZ as described in WO18122225, and/or genomic knock-outs of the E. coli genes comprising any one or more of nanT, poxB, ldhA, adhE, aldB, pflA, pflC, ybiY, ackA and/or pta and with genomic knock-ins of constitutive transcriptional units comprising one or more copies of an L-glutamine-D-fructose-6-phosphate aminotransferase like, e.g., E. coli glmS (UniProt ID P17169, sequence version 04, 23 Jan. 2007), preferably a phosphatase like any one of, e.g., the E. coli genes comprising aphA, Cof, HisB, OtsB, SurE, Yaed, YcjU, YedP, YfbT, YidA, YigB, YihX, YniC, YqaB, YrbL, AppA, Gph, SerB, YbhA, YbiV, YbjL, Yfb, YieH, YjgL, YjjG, YrfG and YbiU or PsMupP from Pseudomonas putida, ScDOG1 from S. cerevisiae or BsAraL from Bacillus subtilis as described in WO18122225 and an acetyl-CoA synthetase like, e.g., acs from E. coli (UniProt ID P27550).

For sialylated oligosaccharide production, the sialic acid production strains were further modified to express an N-acylneuraminate cytidylyltransferase like, e.g., the NeuA enzyme from P. multocida (UniProt ID A0A849CI62) and to express a sialyltransferase like, e.g., the α-2,3-siayltransferase PmultST3 from P. multocida (UniProt ID Q9CLP3) to produce 3′SL or the α-2,6-sialyltransferase PdST6 from P. damselae (UniProt ID 066375) to produce 6′SL. Constitutive transcriptional units of the N-acylneuraminate cytidylyltransferase and the sialyltransferase can be delivered to the mutant strain either via genomic knock-in or via expression plasmids. If the mutant strains producing sialic acid and CMP-sialic acid were intended to make sialylated lactose structures, the strains were additionally modified with genomic knock-outs of the E. coli LacZ, LacY and LacA genes and with a genomic knock-in of a constitutive transcriptional unit for a lactose permease like, e.g., E. coli LacY (UniProt ID P02920). All mutant strains producing sialic acid, CMP-sialic acid and/or sialylated saccharides could optionally be adapted for growth on sucrose via genomic knock-ins of constitutive transcriptional units containing a sucrose transporter like, e.g., CscB from E. coli W (UniProt ID E0IXR1), a fructose kinase like, e.g., Frk originating from Z. mobilis (UniProt ID Q03417) and a sucrose phosphorylase like, e.g., BaSP from B. adolescentis (UniProt ID A0ZZH6).

In another example, the mutant E. coli strains adapted for LNT production as described herein can also be further modified with one or more copies of a glucosamine 6-phosphate N-acetyltransferase like, e.g., GNA1 from S. cerevisiae (UniProt ID P43577), an N-acetylglucosamine 2-epimerase like, e.g., AGE from B. ovatus (UniProt ID A7LVG6) and an N-acetylneuraminate synthase like, e.g., NeuB from N. meningitidis (UniProt ID E0NCD4) and an expression plasmid comprising containing constitutive expression cassettes for the N-acylneuraminate cytidylyltransferase (NeuA) from P. multocida (UniProt ID A0A849CI62) and 1) the α-2,3-sialyltransferase PmultST3 from P. multocida (UniProt ID Q9CLP3) or 2) the α-2,6-sialyltransferase (PdST6) from Photobacterium damselae (UniProt ID 066375) to produce 1) LSTa (Neu5Ac-α2,3-Gal-β1,3-GlcNAc-β1,3-Gal-β1,4-Glc) or 2) LSTb (Gal-β1,3-(Neu5Ac-α2,6)-GlcNAc-β1,3-Gal-β1,4-Glc), respectively. In another example, the mutant E. coli strains adapted for LNnT production as described herein can also be further modified with one or more copies of a glucosamine 6-phosphate N-acetyltransferase like, e.g., GNA1 from S. cerevisiae (UniProt ID P43577), an N-acetylglucosamine 2-epimerase like, e.g., AGE from B. ovatus (UniProt ID A7LVG6) and an N-acetylneuraminate synthase like, e.g., NeuB from N. meningitidis (UniProt ID E0NCD4) and an expression plasmid comprising containing constitutive expression cassettes for the N-acylneuraminate cytidylyltransferase (NeuA) from P. multocida (UniProt ID A0A849CI62) and 1) the α-2,3-sialyltransferase PmultST3 from P. multocida (UniProt ID Q9CLP3) or 2) the α-2,6-sialyltransferase (PdST6) from P. damselae (UniProt ID 066375) to produce 1) LSTd (Neu5Ac-α2,3-Gal-β1,4-GlcNAc-β1,3-Gal-β1,4-Glc) or 2) LSTc (Neu5Ac-α2,6-Gal-β1,4-GlcNAc-β1,3-Gal-β1,4-Glc), respectively.

B. Saccharomyces cerevisiae

### Media and Cultivation

Strains were grown on Synthetic Defined yeast medium with Complete Supplement Mixture (SD CSM) or CSM drop-out (SD CSM-Ura, SD CSM-Trp, SD CSM-His) containing 6.7 g/L Yeast Nitrogen Base without amino acids (YNB w/o AA, Difco), 20 g/L agar (Difco) (solid cultures), 22 g/L glucose monohydrate or 20 g/L lactose and 0.79 g/L CSM or 0.77 g/L CSM-Ura, 0.77 g/L CSM-Trp, or 0.77 g/L CSM-His (MP Biomedicals). As precursor(s) and/or acceptor(s) for saccharide synthesis, compounds like, e.g., galactose, glucose, fructose, fucose, lactose, sialic acid, N-acetyllactosamine (LacNAc), lacto-N-biose (LNB) could be added to the medium. For LBA production, a cofactor like, e.g., pyrroloquinoline quinone (PQQ), FAD, a divalent metal cation, Ca²⁺, Cu²⁺, Mg²⁺, Zn²⁺, heme, heme C and pyridoxal 5′-phosphate may be added.

In general, yeast strains were initially grown on SD CSM plates to obtain single colonies. These plates were grown for 2-3 days at 30° C. Starting from a single colony, a preculture was grown overnight in 5 mL at 30° C., shaking at 200 rpm. Subsequent 125 mL shake flask experiments were inoculated with 2% of this preculture, in 25 mL media. These shake flasks were incubated at 30° C. with an orbital shaking of 200 rpm.

### Strains, Plasmids and Mutations

S. cerevisiae BY4742 created by Brachmann et al. (Yeast (1998) 14:115-32) was used, available in the Euroscarf culture collection. All mutant strains were created by homologous recombination or plasmid transformation using the method of Gietz (Yeast 11:355-360, 1995). Genes were expressed using synthetic constitutive promoters, as described by, e.g., Blazeck (Biotechnology and Bioengineering, Vol. 109, No. 11, 2012), Redden and Alper (Nat. Commun. 2015, 6, 7810), Liu et al. (Microb. Cell Fact. 2020, 19, 38), Xu et al. (Microb. Cell Fact. 2021, 20, 148) and Lee et al. (ACS Synth. Biol. 2015, 4(9), 975-986).

In an example to produce GDP-fucose, a yeast expression plasmid like p2a_2 μ_Fuc (Chan 2013, Plasmid 70, 2-17) can be used for expression of foreign genes in S. cerevisiae. This plasmid contains an ampicillin resistance gene and a bacterial origin of replication to allow for selection and maintenance in E. coli and the 2μ yeast on and the Ura3 selection marker for selection and maintenance in yeast. This plasmid is further modified with constitutive transcriptional units for a lactose permease like, e.g., LAC12 from K. lactis (UniProt ID P07921), a GDP-mannose 4,6-dehydratase like, e.g., gmd from E. coli (UniProt ID POAC88) and a GDP-L-fucose synthase like, e.g., fcl from E. coli (UniProt ID P32055). The yeast expression plasmid p2a_2 μ_Fuc2 can be used as an alternative expression plasmid of the p2a_2 μ_Fuc plasmid comprising next to the ampicillin resistance gene, the bacterial ori, the 2μ yeast on and the Ura3 selection marker constitutive transcriptional units for a lactose permease like, e.g., LAC12 from K. lactis (UniProt ID P07921), a fucose permease like, e.g., fucP from E. coli (UniProt ID P11551) and a bifunctional enzyme with fucose kinase/fucose-1-phosphate guanylyltransferase activity like, e.g., fkp from Bacteroides fragilis (UniProt ID SUV40286.1). To further produce a fucosylated saccharide, the p2a_2 μ_Fuc and its variant the p2a_2 μ_Fuc2, additionally contained a constitutive transcriptional unit for a fucosyltransferase.

In an example to produce UDP-galactose, a yeast expression plasmid can be derived from the pRS420-plasmid series (Christianson et al., 1992, Gene 110: 119-122) containing the HIS3 selection marker and a constitutive transcriptional unit for an UDP-glucose-4-epimerase like, e.g., galE from E. coli (UniProt ID P09147). This plasmid can be further modified with constitutive transcriptional units for a lactose permease like, e.g., LAC12 from K. lactis (UniProt ID P07921) and a galactoside β-1,3-N-acetylglucosaminyltransferase activity like, e.g., lgtA from N. meningitidis (UniProt ID Q9JXQ6) to produce LN3. In an example to further produce LN3-derived oligosaccharides like LNT, the mutant LN3 producing strains were further modified with a constitutive transcriptional unit for an N-acetylglucosamine β-1,3-galactosyltransferase like, e.g., WbgO (Uniprot ID D3QY14) from E. coli O55:H7. In an example for production of LN3 derived oligosaccharides like lacto-N-neotetraose (LNnT, Gal-β1,4-GlcNAc-β1,3-Gal-β1,4-Glc), the mutant LN3 producing strain were further modified with a constitutive transcriptional unit for an N-acetylglucosamine β-1,4-galactosyltransferase like, e.g., LgtB (Uniprot ID Q51116, sequence version 02, 1 Dec. 2000) from N. meningitidis.

In an example to produce sialic acid and CMP-sialic acid, a yeast expression plasmid was derived from the pRS420-plasmid series (Christianson et al., 1992, Gene 110: 119-122) containing the TRP1 selection marker and constitutive transcriptional units for an L-glutamine—D-fructose-6-phosphate aminotransferase like, e.g., E. coli glmS (UniProt ID P17169 (sequence version 04 (23 Jan. 2007)), a phosphatase like, e.g., SurE from E. coli (UniProt ID P0A840), an N-acylglucosamine 2-epimerase like, e.g., AGE from B. ovatus (UniProt ID A7LVG6), an N-acetylneuraminate synthase like, e.g., NeuB from N. meningitidis (UniProt ID E0NCD4) and an N-acylneuraminate cytidylyltransferase like, e.g., NeuA from P. multocida (UniProt A0A849CI62). Optionally, a constitutive transcriptional unit for a glucosamine 6-phosphate N-acetyltransferase like, e.g., GNA1 from S. cerevisiae (UniProt ID P43577) was added as well. In an example to produce a sialylated saccharide, the plasmid further comprised constitutive transcriptional units for a lactose permease like, e.g., LAC12 from K. lactis (UniProt ID P07921), and a sialyltransferase.

Preferably but not necessarily, any one or more of the glycosyltransferases and/or the proteins involved in nucleotide-activated sugar synthesis were N- and/or C-terminally fused to a SUMOstar tag (e.g., obtained from pYSUMOstar, Life Sensors, Malvern, PA) to enhance their solubility.

Optionally, the mutant yeast strains were modified with a genomic knock-in of a constitutive transcriptional unit encoding a chaperone protein like, e.g., Hsp31, Hsp32, Hsp33, Sno4, Kar2, Ssβ1, Sse1, Sse2, Ssα1, Ssα2, Ssa3, Ssa4, Ssb2, Ecm10, Ssc1, Ssq1, Ssz1, Lhs1, Hsp82, Hsc82, Hsp78, Hsp104, Tcp1, Cct4, Cct8, Cct2, Cct3, Cct5, Cct6 or Cct7 (Gong et al., 2009, Mol. Syst. Biol. 5: 275). Plasmids were maintained in the host E. coli DH5α (F⁻, phi80dlacZdeltaM15, delta(lacZYA-argF)U169, deoR, recA1, endA1, hsdR17(rk⁻, mk⁺), phoA, supE44, lambda⁻, thi-1, gyrA96, relA1) bought from Invitrogen.

C. Bacillus subtilis

### Media and Cultivation

Two media are used to cultivate B. subtilis: i.e., a complex medium like a rich Luria Broth (LB) and a minimal medium for shake flask cultures. The LB medium consisted of 1% tryptone peptone (Difco), 0.5% yeast extract (Difco) and 0.5% sodium chloride (VWR). Luria Broth agar (LBA) plates consisted of the LB media, with 12 g/L agar (Difco) added. The minimal medium contained 2.00 g/L (NH₄)₂SO₄, 7.5 g/L KH₂PO₄, 17.5 g/L K₂HPO₄, 1.25 g/L Na-citrate, 0.25 g/L MgSO₄·7H₂O, 0.05 g/L tryptophan, from 10 up to 30 g/L glucose (or another carbon source including but not limited to fructose, maltose, sucrose, glycerol and maltotriose), 10 mL/L trace element mix and 10 mL/L Fe-citrate solution. The medium was set to a pH of 7 with 1 M KOH. As precursor(s) and/or acceptor(s) for saccharide synthesis, compounds like, e.g., galactose, glucose, fructose, fucose, lactose, sialic acid, LacNAc, LNB could be added to the medium. For LBA production, a cofactor like, e.g., PQQ, FAD, a divalent metal cation, Ca²⁺, Cu²⁺, Mg²⁺, Zn²⁺, heme, heme C and pyridoxal 5′-phosphate may be added. The trace element mix consisted of 0.735 g/L CaCl₂·2H₂O, 0.1 g/L MnCl₂·2H₂O, 0.033 g/L CuCl₂·2H₂O, 0.06 g/L CoCl₂·6H₂O, 0.17 g/L ZnCl₂, 0.0311 g/L H₃BO₄, 0.4 g/L Na₂EDTA·2H₂O and 0.06 g/L Na₂MoO₄. The Fe-citrate solution contained 0.135 g/L FeCl₃·6H₂O, 1 g/L Na-citrate (Hoch 1973 PMC1212887). Complex medium, e.g., LB, was sterilized by autoclaving (121° C., 21 minutes) and minimal medium by filtration (0.22 μm Sartorius). When necessary, the medium was made selective by adding an antibiotic.

B. subtilis strains were initially grown on LB agar to obtain single colonies. These plates were grown overnight at 37° C. Starting from a single colony, a preculture was grown overnight in 5 mL at 37° C., shaking at 200 rpm. Subsequent 125 mL shake flask experiments were inoculated with 2% of this preculture, in 25 mL media. These shake flasks were incubated at 37° C. with an orbital shaking of 200 rpm for 72 hours, or shorter or longer. At the end of the cultivation experiment samples were taken to measure the supernatant concentration (extracellular sugar concentrations, after 5 minutes spinning down the cells), or by boiling the culture broth for 15 minutes at 90° C. or for 60 minutes at 60° C. before spinning down the cells (=whole broth concentration, i.e., intra- and extracellular sugar concentrations).

### Strains, Plasmids and Mutations

B. subtilis 168 is used as available at the Bacillus Genetic Stock Center (Ohio, USA). Plasmids for gene deletion via Cre/lox are constructed as described by Yan et al. (Appl & Environm. Microbial, September 2008, p5556-5562). Gene disruption is done via homologous recombination with linear DNA and transformation via the electroporation as described by Xue et al. (J. Microb. Meth. 34 (1999) 183-191). The method of gene knockouts is described by Liu et al. (Metab. Engine. 24 (2014) 61-69). Integrative vectors as described by Popp et al. (Sci. Rep., 2017, 7, 15158) are used as expression vector and could be further used for genomic integrations if necessary. A suitable promoter for expression can be derived from the part repository (iGem): sequence id: BBa_K143012, BBa_K823000, BBa_K823002 or BBa_K823003. Cloning can be performed using Gibson Assembly, Golden Gate assembly, Cliva assembly, LCR or restriction ligation.

In an example for the production of lactose-based oligosaccharides, B. subtilis mutant strains are created to contain a gene coding for a lactose importer (such as, e.g., the E. coli lacY with UniProt ID P02920).

In an example for the production of LN3, the B. subtilis strain is modified with a genomic knock-in of constitutive transcriptional units comprising a lactose importer (such as, e.g., the E. coli lacY with UniProt ID P02920) and a galactoside β-1,3-N-acetylglucosaminyltransferase like, e.g., LgtA from N. meningitidis (UniProt ID Q9JXQ6). For LNT production, the LN3 producing strain is further modified with a constitutive transcriptional unit for an N-acetylglucosamine β-1,3-galactosyltransferase like, e.g., WbgO from E. coli O55:H7 (UniProt ID D3QY14). For LNnT production, the LN3 producing strain is further modified with a constitutive transcriptional unit for an N-acetylglucosamine β-1,4-galactosyltransferase like, e.g., LgtB from N. meningitidis (UniProt ID Q51116, sequence version 02, 1 Dec. 2000). The N-acetylglucosamine β-1,3-galactosyltransferase and the N-acetylglucosamine β-1,4-galactosyltransferase can be delivered to the strain either via genomic knock-in or from an expression plasmid.

To produce a fucosylated saccharide, the B. subtilis strains are modified with a constitutive transcriptional unit for a fucosyltransferase.

In an example for sialic acid production, a mutant B. subtilis strain is created by overexpressing a fructose-6-P-aminotransferase like the native fructose-6-P-aminotransferase glmS (UniProt ID P0CI73) to enhance the intracellular glucosamine-6-phosphate pool. Further on, the enzymatic activities of the genes nagA, nagB and gamA are disrupted by genetic knockouts and a glucosamine-6-P-aminotransferase like, e.g., GNA1 from S. cerevisiae (UniProt ID P43577), an N-acetylglucosamine-2-epimerase like, e.g., from B. ovatus (UniProt ID A7LVG6) and an N-acetylneuraminate synthase like, e.g., from N. meningitidis (UniProt ID E0NCD4) are overexpressed on the genome. To allow sialylated saccharide production, the sialic acid producing strain is further modified with a constitutive transcriptional unit comprising an N-acylneuraminate cytidylyltransferase like, e.g., the NeuA enzyme from P. multocida (UniProt ID A0A849CI62), and a sialyltransferase.

For growth on sucrose, the mutant strains can additionally be modified with genomic knock-ins of constitutive transcriptional units comprising the sucrose transporter (CscB) from E. coli W (UniProt ID EOIXR1), the fructose kinase (Frk) from Z. mobilis (UniProt ID Q03417) and the sucrose phosphorylase (BaSP) from B. adolescentis (UniProt ID AOZZH6).

D. Corynebacterium glutamicum

### Media and Cultivation

Two different media are used, namely complex medium like, e.g., a rich tryptone-yeast extract (TY) medium, and a minimal medium for shake flask (MMsf). The minimal medium uses a 1000× stock trace element mix. Trace element mix consisted of 10 g/L CaCl₂), 10 g/L FeSO₄·7H₂O, 10 g/L MnSO₄·H₂O, 1 g/L ZnSO₄·7H₂O, 0.2 g/L CuSO₄, 0.02 g/L NiCl₂·6H₂O, 0.2 g/L biotin (pH 7) and 0.03 g/L protocatechuic acid. The minimal medium for the shake flasks (MMsf) experiments contained 20 g/L (NH₄)₂SO₄, 5 g/L urea, 1 g/L KH₂PO₄, 1 g/L K₂HPO₄, 0.25 g/L MgSO₄·7H₂O, 42 g/L MOPS, from 10 up to 30 g/L glucose or another carbon source including but not limited to fructose, maltose, sucrose, glycerol and maltotriose when specified in the examples and 1 ml/L trace element mix. As precursor(s) and/or acceptor(s) for saccharide synthesis, compounds like, e.g., galactose, glucose, fructose, fucose, lactose, sialic acid, LacNAc, LNB could be added to the medium. For LBA production, a cofactor like, e.g., pyrroloquinoline quinone (PQQ), FAD, a divalent metal cation, Ca²⁺, Cu²⁺, Mg²⁺, Zn²⁺, heme, heme C and pyridoxal 5′-phosphate may be added. The TY medium consisted of 1.6% tryptone (Difco, Erembodegem, Belgium), 1% yeast extract (Difco) and 0.5% sodium chloride (VWR. Leuven, Belgium). TY agar (TYA) plates consisted of the TY media, with 12 g/L agar (Difco, Erembodegem, Belgium) added. Complex medium, e.g., TY, was sterilized by autoclaving (121° C., 21 min) and minimal medium by filtration (0.22 μm Sartorius). When necessary, the medium was made selective by adding an antibiotic.

A preculture was started from a cryovial or a single colony from a TY plate, in 6 mL TY and was incubated overnight at 37° C. on an orbital shaker at 200 rpm. Subsequent 125 mL shake flask experiments were inoculated with 2% of this preculture, in 25 mL MMsf medium. These shake flasks were incubated at 37° C. with an orbital shaking of 200 rpm for 72 hours, or shorter or longer. At the end of the cultivation experiment samples were taken to measure the supernatant concentration (extracellular sugar concentrations, after 5 minutes spinning down the cells), or by boiling the culture broth for 15 minutes at 90° C. or for 60 minutes at 60° C. before spinning down the cells (=whole broth concentration, i.e., intra- and extracellular sugar concentrations).

### Strains and Mutations

Corynebacterium glutamicum was used as available at the American Type Culture Collection (ATCC 13032). Integrative plasmid vectors were made using the Cre/loxP technique as described by Suzuki et al. (Appl. Microbiol. Biotechnol., 2005 April, 67(2):225-33) and temperature-sensitive shuttle vectors as described by Okibe et al. (Journal of Microbiological Methods 85, 2011, 155-163) are constructed for gene deletions, mutations and insertions. Suitable promoters for (heterologous) gene expression can be derived from Yim et al. (Biotechnol. Bioeng., 2013 November, 110(11):2959-69). Cloning can be performed using Gibson Assembly, Golden Gate assembly, Cliva assembly, LCR or restriction ligation.

In an example for production of lactose-based oligosaccharides, C. glutamicum mutant strains are created to contain a gene coding for a lactose importer (such as, e.g., the E. coli lacY with UniProt ID P02920).

In an example for the production of LN3, the C. glutamicum strain is modified with a genomic knock-in of constitutive expression units comprising a lactose importer (such as, e.g., the E. coli lacY with UniProt ID P02920) and a galactoside β-1,3-N-acetylglucosaminyltransferase like, e.g., LgtA from N. meningitidis (UniProt ID Q9JXQ6). For LNT production, the LN3 producing strain is further modified with a constitutive transcriptional unit for an N-acetylglucosamine β-1,3-galactosyltransferase like, e.g., WbgO from E. coli O55:H7 (UniProt ID D3QY14). For LNnT production, the LN3 producing strain is further modified with a constitutive transcriptional unit for an N-acetylglucosamine β-1,4-galactosyltransferase like, e.g., LgtB from N. meningitidis (UniProt ID Q51116, sequence version 02, 1 Dec. 2000). The N-acetylglucosamine β-1,3-galactosyltransferase and the N-acetylglucosamine β-1,4-galactosyltransferase can be delivered to the strain either via genomic knock-in or from an expression plasmid.

To further produce a fucosylated saccharide, the mutant C. glutamicum strains are further modified with a constitutive transcriptional unit for a fucosyltransferase.

In an example for sialic acid production, a mutant C. glutamicum strain is created by overexpressing a fructose-6-P-aminotransferase like the native fructose-6-P-aminotransferase glmS (UniProt ID Q8NND3, sequence version 03, 23 Jan. 2007) to enhance the intracellular glucosamine-6-phosphate pool. Further on, the enzymatic activities of the genes nagA, nagB and gamA are disrupted by genetic knockouts and a glucosamine-6-P-aminotransferase like, e.g., GNA1 from S. cerevisiae (UniProt ID P43577), an N-acetylglucosamine-2-epimerase like, e.g., from B. ovatus (UniProt ID A7LVG6) and an N-acetylneuraminate synthase like, e.g., from N. meningitidis (UniProt ID E0NCD4) are overexpressed on the genome. To allow sialylated saccharide production, the sialic acid producing strain is further modified with a constitutive transcriptional unit comprising an N-acylneuraminate cytidylyltransferase like, e.g., the NeuA enzyme from P. multocida (UniProt ID A0A849CI62), and a sialyltransferase.

For growth on sucrose, the mutant strains can additionally be modified with genomic knock-ins of constitutive transcriptional units comprising the sucrose transporter (CscB) from E. coli W (UniProt ID EOIXR1), the fructose kinase (Frk) from Z. mobilis (UniProt ID Q03417) and the sucrose phosphorylase (BaSP) from B. adolescentis (UniProt ID AOZZH6).

### E. Optical Density

Cell density of the cultures was frequently monitored by measuring optical density at 600 nm (Implen Nanophotometer NP80, Westburg, Belgium or with a Spark 10M microplate reader, Tecan, Switzerland). The maximum growth speed (mumax) was calculated based on the observed optical densities at 600 nm using the R package grofit.

### F. Growth Rate Speed Measurement

The maximal growth rate (pMax) was calculated based on the observed optical densities at 600 nm using the R package grofit.

### G. Heterologous and Homologous Expression

Genes that needed to be expressed, be it from a plasmid or from the genome were synthetically synthetized with one of the following companies: IDT or Twist Bioscience. Proteins described in present disclosure are summarized in Table 1. Unless stated otherwise, the UniProt IDs of the proteins described correspond to their sequence version 01 as present in the UniProt Database version release 2021_03 of 9 Jun. 2021. Expression could be further facilitated by optimizing the codon usage to the codon usage of the expression host. Genes were optimized using the tools of the supplier.

TABLE 1
Overview of proteins with corresponding UniProt IDs (sequence version 01,
UniProt Database 2021_03 of 9 Jun. 2021) as described in present disclosure

| | | | | Country of origin of |
|---|---|---|---|---|
| | | | | digital sequence |
| UniProt ID | Name | Organism | Origin | information |
| P27550 | acs | Escherichia coli K-12 MG1655 | Synthetic | USA |
| A7LVG6 | AGE | Bacteroides ovatus | Synthetic | USA |
| A0ZZH6 | BaSP | Bifidobacterium adolescentis | Synthetic | Germany |
| E0IXR1 | cscB | Escherichia coli W | Synthetic | USA |
| A0A1B1EJ52 | dehydrogenase | Vibrio natriegens | Synthetic | USA |
| P32055* | fcl | Escherichia coli K-12 MG1655 | Synthetic | USA |
| SUV40286.1 | fkp | Bacteroides fragilis | Synthetic | United Kingdom |
| Q03417 | Frk | Zymomonas mobilis | Synthetic | United Kingdom |
| P11551** | fucP | Escherichia coli K-12 MG1655 | Synthetic | USA |
| P09147 | galE | Escherichia coli K-12 MG1655 | Synthetic | USA |
| P15877*** | gcd | Escherichia coli K-12 MG1655 | Synthetic | USA |
| A0A1Y2NTL5 | gdhB_2 | Streptomyces fradiae | Synthetic | Unknown |
| P31120**** | glmM | Escherichia coli K-12 MG1655 | Synthetic | USA |
| P0CI73 | glmS | Bacillus subtilis | Synthetic | USA |
| Q8NND3***** | glmS | Corynebacterium glutamicum | Synthetic | USA |
| P17169° | glmS | Escherichia coli K-12 MG1655 | Synthetic | USA |
| P0ACC7 | glmU | Escherichia coli K-12 MG1655 | Synthetic | USA |
| P0AC88 | gmd | Escherichia coli K-12 MG1655 | Synthetic | USA |
| P43577 | GNA1 | Saccharomyces cerevisiae | Synthetic | USA |
| Q91WG8 | GNE | Mus musculus | Synthetic | USA |
| O30511 | HpFucT | Helicobacter pylori UA1234 | Synthetic | Canada |
| Q9X435 | HpFutC | Helicobacter pylori UA1234 | Synthetic | Canada |
| P07921 | LAC12 | Kluyveromyces lactis | Synthetic | USA |
| P02920 | LacY | Escherichia coli K-12 MG1655 | Synthetic | USA |
| Q9JXQ6 | LgtA | Neisseria meningitidis MC58 | Synthetic | United Kingdom |
| Q51116°° | LgtB | Neisseria meningitidis MC58 | Synthetic | United Kingdom |
| P00946 | manA | Escherichia coli K-12 MG1655 | Synthetic | USA |
| P24175 | manB | Escherichia coli K-12 MG1655 | Synthetic | USA |
| P24174°°° | manC | Escherichia coli K-12 MG1655 | Synthetic | USA |
| Q8A712 | NANP | Bacteroides thetaiotaomicron | Synthetic | Unknown |
| K9NPH9 | NANS | Pseudomonas sp. UW4 | Synthetic | Unknown |
| Q99KK2°°°° | NeuA | Mus musculus | Synthetic | USA |
| A0A849CI62 | NeuA | Pasteurella multocida | Synthetic | USA |
| E0NCD4 | NeuB | Neisseria meningitidis | Synthetic | United Kingdom |
| Q93MP8 | NeuC | Campylobacter jejuni | Synthetic | USA |
| O66375 | PdST6 | Photobacterium damselae | Synthetic | Japan |
| Q9CLP3 | PmultST3 | Pasteurella multocida | Synthetic | USA |
| P0A840 | SurE | Escherichia coli K-12 MG1655 | Synthetic | USA |
| D3QY14 | wbgO | Escherichia coli O55: H7 | Synthetic | Germany |
| P75804 | ylil | Escherichia coli K-12 MG1655 | Synthetic | USA |

*Sequence version 02 (1 Nov. 1997) as present in the UniProt Database 2021_03 of 9 Jun. 2021;
**Sequence version 03 (23 Jan. 2007) as present in the UniProt Database 2021_03 of 9 Jun. 2021;
***Sequence version 03 (2 Dec. 2020) as present in the UniProt Database 2021_03 of 9 Jun. 2021;
****Sequence version 03 (23 Jan. 2007) as present in the UniProt Database 2021_03 of 9 Jun. 2021;
*****Sequence version 02 (23 Jan. 2007) as present in the UniProt Database 2021_03 of 9 Jun. 2021;
°Sequence version 04 (23 Jan. 2007) as present in the UniProt Database 2021_03 of 9 Jun. 2021;
°°Sequence version 02 (1 Dec. 2000) as present in the UniProt Database 2021_03 of 9 Jun. 2021;
°°°Sequence version 03 (19 Jul. 2003) as present in the UniProt Database 2021_03 of 9 Jun. 2021;
°°°°Sequence version 02 (1 Feb. 2005) as present in the UniProt Database 2021_03 of 9 Jun. 2021

### H. Analytical Analysis

Standards such as but not limited to sucrose, lactose, 3′SL, 6′SL, lacto-N-triose II (LN3), lacto-N-tetraose (LNT), lacto-N-neo-tetraose (LNnT), LNFP-I, LNFP-II, LNFP-III, LNFP-V, LNFP-VI, LSTa, LSTc and LSTd were purchased from Carbosynth (UK), Elicityl (France) and IsoSep (Sweden). Other compounds were analyzed with in-house made standards.

Neutral oligosaccharides were analyzed on a Waters Acquity H-class UPLC with Evaporative Light Scattering Detector (ELSD) or a Refractive Index (RI) detection. A volume of 0.7 μL sample was injected on a Waters Acquity UPLC BEH Amide column (2.1×100 mm; 130 Å; 1.7 μm) column with an Acquity UPLC BEH Amide VanGuard column, 130 Å, 2.1×5 mm. The column temperature was 50° C. The mobile phase consisted of a ¼ water and ¾ acetonitrile solution to which 0.2% triethylamine was added. The method was isocratic with a flow of 0.130 mL/min. The ELSD detector had a drift tube temperature of 50° C. and the N2 gas pressure was 50 psi, the gain 200 and the data rate 10 pps. The temperature of the RI detector was set at 35° C.

Sialylated oligosaccharides were analyzed on a Waters Acquity H-class UPLC with Refractive Index (RI) detection. A volume of 0.5 μL sample was injected on a Waters Acquity UPLC BEH Amide column (2.1×100 mm; 130 Å; 1.7 μm). The column temperature was 50° C. The mobile phase consisted of a mixture of 70% acetonitrile, 26% ammonium acetate buffer (150 mM) and 4% methanol to which 0.05% pyrrolidine was added. The method was isocratic with a flow of 0.150 mL/min. The temperature of the RI detector was set at 35° C.

Both neutral and sialylated sugars were analyzed on a Waters Acquity H-class UPLC with Refractive Index (RI) detection. A volume of 0.5 μL sample was injected on a Waters Acquity UPLC BEH Amide column (2.1×100 mm; 130 Å; 1.7 μm). The column temperature was 50° C. The mobile phase consisted of a mixture of 72% acetonitrile and 28% ammonium acetate buffer (100 mM) to which 0.1% triethylamine was added. The method was isocratic with a flow of 0.260 mL/min. The temperature of the RI detector was set at 35° C.

For analysis on a mass spectrometer, a Waters Xevo TQ-MS with Electron Spray Ionization (ESI) was used with a desolvation temperature of 450° C., a nitrogen desolvation gas flow of 650 L/h and a cone voltage of 20 V. The MS was operated in selected ion monitoring (SIM) in negative mode for all oligosaccharides. Separation was performed on a Waters Acquity UPLC with a Thermo Hypercarb column (2.1×100 mm; 3 μm) on 35° C. A gradient was used wherein eluent A was ultrapure water with 0.1% formic acid and wherein eluent B was acetonitrile with 0.1% formic acid. The oligosaccharides were separated in 55 minutes using the following gradient: an initial increase from 2 to 12% of eluent B over 21 minutes, a second increase from 12 to 40% of eluent B over 11 minutes and a third increase from 40 to 100% of eluent B over 5 minutes. As a washing step, 100% of eluent B was used for 5 minutes. For column equilibration, the initial condition of 2% of eluent B was restored in 1 minute and maintained for 12 minutes.

Both neutral and sialylated sugars at low concentrations (below 50 mg/L) were analyzed on a Dionex HPAEC system with pulsed amperometric detection (PAD). A volume of 5 μL of sample was injected on a Dionex CarboPac PA200 column 4×250 mm with a Dionex CarboPac PA200 guard column 4×50 mm. The column temperature was set to 30° C. A gradient was used wherein eluent A was deionized water, wherein eluent B was 200 mM Sodium hydroxide and wherein eluent C was 500 mM Sodium acetate. The oligosaccharides were separated in 60 minutes while maintaining a constant ratio of 25% of eluent B using the following gradient: an initial isocratic step maintained for 10 minutes of 75% of eluent A, an initial increase from 0 to 4% of eluent C over 8 minutes, a second isocratic step maintained for 6 minutes of 71% of eluent A and 4% of eluent C, a second increase from 4 to 12% of eluent C over 2.6 minutes, a third isocratic step maintained for 3.4 minutes of 63% of eluent A and 12% of eluent C and a third increase from 12 to 48% of eluent C over 5 minutes. As a washing step, 48% of eluent C was used for 3 minutes. For column equilibration, the initial condition of 75% of eluent A and 0% of eluent C was restored in 1 minute and maintained for 11 minutes. The applied flow was 0.5 mL/min.

Lactobionic acid was analysed on a Dionex HPAEC system with pulsed amperometric detection (PAD). A volume of 5 μL of sample was injected on a Dionex CarboPac PA01 column 2×250 mm with a Dionex CarboPac PA01 guard column 4×50 mm. The column temperature was set to 30° C. A gradient was used wherein eluent A was ultrapure water, eluent B was 200 mM Sodium hydroxide and eluent C was 500 mM Sodium acetate. Total gradient time was 41 minutes and started with 50% B and 5% C in the first 7 minutes. During the next 18 minutes, concentration slowly changed to 50% B and 40% C at 25 minutes, followed by a rinse step with 100% C for 7 minutes. For column equilibration, the initial condition with 50% B and 5% C was restored in 9 minutes. The applied flow was 0.25 mL/min.

### Example 2. Reduced Production of Lactobionic Acid (LBA) in Modified E. coli Hosts Wherein the LBA Synthesis is Knocked Out

Mutant E. coli strains for the production of 2′FL, LNT, LNnT, 3′SL or 6′SL were engineered as described in Example 1. These strains were further modified with a genomic knock-out of the native quinoprotein glucose dehydrogenase gene (GeneID: 944830), encoding the gcd enzyme (UniProt ID P15877, sequence version 03 (2 Dec. 2020)). In a next step, these mutant strains were further modified with a genomic knock-out for the native aldose sugar dehydrogenase gene (GeneID: 945467), encoding the ylil enzyme (UniProt ID P75804).

All novel strains together with the corresponding reference strains, having the same genetic make-up but having the native gcd gene and ylil gene still present, were evaluated in a growth experiment according to the culture conditions provided in Example 1, in which the strains were cultivated in minimal medium supplemented with 30 g/L sucrose, 20 g/L lactose and 3.30E-4 g/L pyrroloquinoline quinone (PQQ). The strains were grown in three biological replicates in a 96-well plate. After 72 hours of incubation, the culture broth was harvested, and lactobionic acid (LBA) production was analyzed on UPLC. Also, the maximum growth speed (Mumax, h⁻¹) was determined as provided in Example 1.

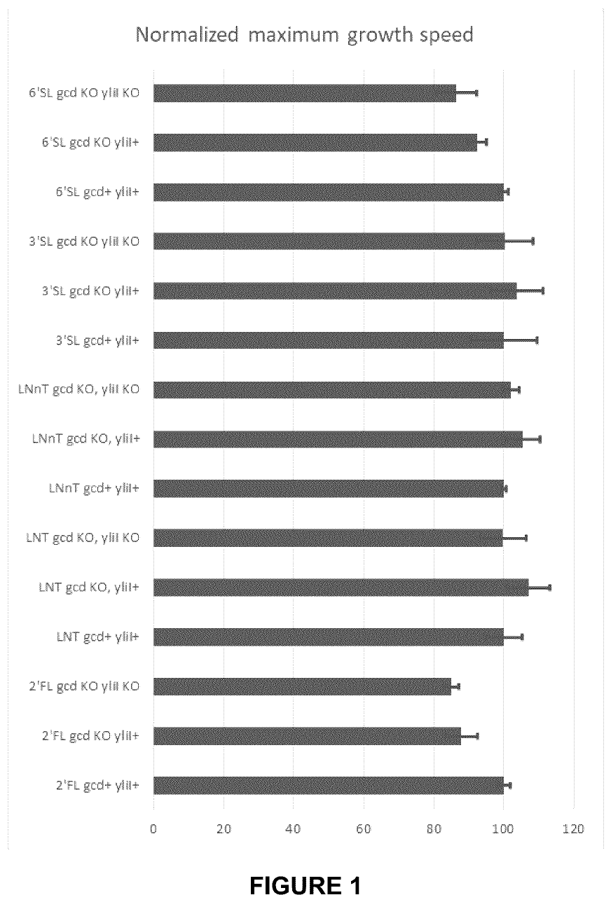

As shown in Table 2, each strain having a gcd knock-out and a native ylil gene demonstrated a reduced LBA production compared to its respective reference strain having the same genetic make-up and natively expressing gcd and ylil. For the LNT, LNnT, 3′SL and 6′SL production strains having a gcd knock-out the LBA production was even reduced below detection limits. For the 2′FL strain adding the ylil knock-out on top of the gcd knock-out resulted in a further lowered LBA production compared to when only the gcd knock-out was made. A knock-out of the gcd gene or a knock-out of both the gcd and ylil genes did not affect the 2′FL, LNT, LNnT, 3′SL or 6′SL production in the respective strains (Results not shown). FIG. 1 demonstrates that the genomic knock-out of gcd and the genomic knock-out of both gcd and ylil had a small effect on the growth speed of the mutant strains compared to the reference strains expressing the native gcd and ylil gene.

TABLE 2
Averaged (n = 3) production of lactobionic acid (LBA) in modified
E. coli strains engineered for production of 2′FL, LNT, LNnT,
3′SL or 6′SL as described in Example 1 and having native gcd
and ylil expression (reference strain, gcd+ ylil+) or having
a gcd genomic knock-out (gcd KO, ylil+) or having a gcd genomic
knock-out and a ylil genomic knock-out (gcd KO, ylil KO) in a growth
experiment according to the culture conditions provided in Example
1, in which the culture medium was supplemented with 30 g/L sucrose,
20 g/L lactose and 3.30E−4 g/L pyrroloquinoline quinone (PQQ).

| Saccharide | | | LBA (g/L) |
|---|---|---|---|
| produced | LBA (g/L) | LBA (g/L) | in mutant |
| by the | in reference | in mutant | strain |
| E. coli | strain (gcd+ | strain (gcd | (gcd KO, |
| strains | ylil+) | KO, ylil+) | ylil KO) |
| 2′FL | 2.917 | 0.712 | 0.053 |
| LNT | 0.196 | N.D.* | N.D. |
| LNnT | 0.305 | N.D. | N.D. |
| 3′SL | 0.011 | N.D. | N.D. |
| 6′SL | 0.016 | N.D. | N.D. |

*N.D. = Not detectable

### Example 3. Production of 3′SL or 6′SL in Modified E. coli Hosts when Evaluated in a Fed-Batch Fermentation Process with Sucrose and Lactose

The mutant E. coli strains modified for production of 3′SL or 6′SL and having a genomic knock-out of both the gcd and the ylil gene (gcd KO, ylil KO) as described in Example 2 were selected for further evaluation in fed-batch fermentation processes. Also, fed-batch fermentation processes were set up with the respective reference strains having the same genetic make-up but having expression of the gcd and the ylil gene (gcd+, ylil+). Fed-batch fermentations at bioreactor scale were performed as described in Example 1. Sucrose was used as a carbon source and lactose was added in the batch medium. During fed-batch, sucrose was added via an additional feed. In contrast to the cultivation experiments that are described herein and wherein only end samples were taken at the end of cultivation (i.e., 72 hours as described herein), regular broth samples were taken at several time points during the fermentation process and the 3′SL or 6′SL produced was measured using UPLC as described in Example 2. For each strain tested, two independent fermentation processes were performed and the measured 3′SL or 6′SL concentration was averaged over all biological replicates and then normalized to the averaged 3′SL or 6′SL concentration of the respective reference strain.

The experiment demonstrated that the averaged 3′SL titer measured at the end of the fed-batch fermentations with the 3′SL strain having the gcd and ylil knock-outs (gcd KO, ylil KO) was 97.1% of the averaged 3′SL titer measured at the end of the fed-batch fermentations with the 3′SL reference strain (gcd+, ylil+). Furthermore, the averaged 6′SL titer measured at the end of the fed-batch fermentations with the 6′SL strain having the gcd and ylil knock-outs (gcd KO, ylil KO) was 99.7% of the averaged 6′SL titer measured at the end of the fed-batch fermentations with the 6′SL reference strain (gcd+, ylil+). Summarized, the 3′SL or 6′SL titers were not affected in the respective 3′SL or 6′SL production strains by the knock-outs of the gcd and the yliI gene.

### Example 4. Production of Oligosaccharides in Modified E. coli Hosts when Evaluated in a Fed-Batch Fermentation Process with Sucrose and Lactose

The mutant E. coli strains modified for production of 2′FL, LNT or LNnT and having a genomic knock-out of both the gcd and the ylil gene (gcd KO, ylil KO) as described in Example 2 are selected for further evaluation in fed-batch fermentation processes. Also, fed-batch fermentation processes are set up with the respective reference strains having the same genetic make-up but having expression of the gcd and the ylil gene (gcd+, ylil+). Fed-batch fermentations at bioreactor scale are performed as described in Example 1. Sucrose is used as a carbon source and lactose is added in the batch medium. During fed-batch, sucrose is added via an additional feed. In contrast to the cultivation experiments that are described herein and wherein only end samples were taken at the end of cultivation (i.e., 72 hours as described herein), regular broth samples are taken at several time points during the fermentation process and the 2′FL, LNT or LNnT titer produced in the respective strains is measured using UPLC as described in Example 1.

### Example 5. Production of Oligosaccharides in Modified E. coli Hosts when Evaluated in a Fed-Batch Fermentation Process with Sucrose and Lactose

The mutant E. coli strains modified for production of 3-FL, DiFL, LSTa, LSTb, LSTc or LSTd as described in Example 1 are further modified to have genetic knock-outs of both the gcd and the ylil gene (gcd KO, ylil KO). Each novel strain is able to produce 3-FL, diFL, LSTa, LSTb, LSTc or LSTd in similar amount as the respective reference strains still having expression of the gcd and yliI genes. The novel strains are selected for further evaluation in fed-batch fermentation processes. Also, fed-batch fermentation processes are set up with the respective reference strains having the same genetic make-up but having expression of the gcd and the ylil gene (gcd+, ylil+). Fed-batch fermentations at bioreactor scale are performed as described in Example 1. Sucrose is used as a carbon source and lactose is added in the batch medium. During fed-batch, sucrose is added via an additional feed. In contrast to the cultivation experiments that are described herein and wherein only end samples were taken at the end of cultivation (i.e., 72 hours as described herein), regular broth samples are taken at several time points during the fermentation process and the 3-FL, diFL, LSTa, LSTb, LSTc or LSTd titer produced in the respective strains is measured using UPLC as described in Example 1.

### Example 6. Evaluation of Production of a Glycosylated Form of LBA in a Modified E. coli Host

The mutant E. coli strains modified for production of 2′FL, 3′SL or 6′SL and modified with a genomic knock-out of both the gcd and ylil gene (gcd KO, ylil KO) as described in Example 2 and the mutant E. coli strains modified for production of 3-FL, DiFL, LSTa, LSTb, LSTc or LSTd and modified with a genomic knock-out of both the gcd and ylil gene (gcd KO, ylil KO) as described in Example 5 are selected for further evaluation in fed-batch fermentation processes. Fed-batch fermentations at bioreactor scale are performed as described in Example 1. Sucrose is used as a carbon source and lactose and PQQ are added in the batch medium. During fed-batch, sucrose is added via an additional feed. In contrast to the cultivation experiments that are described herein and wherein only end samples were taken at the end of cultivation (i.e., 72 hours as described herein), regular broth samples are taken at several time points during the fermentation process and the 2′FL, 3′SL, 6′SL, 3-FL, diFL, LSTa, LSTb, LSTc, LSTd, LBA, fucosylated LBA or sialylated LBA titer produced in the respective strains is measured using UPLC as described in Example 1.

### Example 7. Rendering Genes Less Functional in a Modified Host

Rendering genes less functional is a common practice in biotechnology. As described above there are several techniques to lower expression or render a gene less functional (such as the usage of siRNA, CrispR interference, RNAi, miRNA, asRNA, mutating genes, knocking-out genes, transposon mutagenesis, etc.).

CrispR interference is one of the newest techniques in rendering genes less functional. It entails the design of an sgRNA that recognizes the gene of interest (the gene to be rendered less functional) and the expression of a mutated RNA-guided DNA endonuclease enzyme that lost its endonuclease activity (e.g., the dCas9 protein). The sgRNA is composed of a base pairing region mostly existing of 20 nucleotides downstream or upstream next to a PAM region (e.g., NGG in the case of dCas9). The base pairing region is complementary to a region into the target gene. The closer the base pairing region binds to the 5′ end of the gene target, the better the repression. To avoid off target repression, the base pairing region is BLASTed against the genome, ensuring there are no other regions complementary to the base pairing region apart from the gene of interest. Examples of design tools are described by Doench et. al. 2016 [Nature Biotechnology volume 34, pages 184-191 (2016)] or Labun et al. (2019) [Nucleic Acids Research volume 47, pages W171-W174 (2019)], but is also provided by most synthetic DNA providers.

Both dCas9 and the sgRNA are expressed in the cell according to the methods described in Example 1. Preferentially both are expressed from the genome, ensuring stable expression over several generations.

### Example 8. Evaluation of Lactobionic Acid (LBA) Production in Modified E. coli Hosts Wherein the LBA Synthesis is Rendered Less Functional

Mutant E. coli strains for the production of 2′FL, LNT, LNnT, 3′SL or 6′SL are engineered as described in Example 1. These strains are further modified to have the quinoprotein glucose dehydrogenase gene (GeneID: 944830), encoding the gcd enzyme (UniProt ID P15877), and the aldose sugar dehydrogenase gene (GeneID: 945467), encoding the ylil enzyme (UniProt ID P75804) rendered less functional in their cells to lower or eliminate the production of lactobionic acid (LBA) when grown on medium containing lactose and pyrroloquinoline quinone (PQQ). As an example, both the gcd gene and yiI gene are rendered less functional by using the CrispRi technique as described in Example 7. The sgRNA used for the gcd gene (SEQ ID NO: 01) and the sgRNA used for the ylI gene (SEQ ID NO: 02) both have CGG as a PAM sequence. The sequences are expressed by means of a constitutive promoter sequence as described in Example 1. The novel strains are evaluated in a growth experiment according to the culture conditions provided in Example 1, in which the strains were cultivated in minimal medium supplemented with 30 g/L sucrose, 20 g/L lactose and 3.30E-4 g/L pyrroloquinoline quinone (PQQ). The strains are grown in three biological replicates in a 96-well plate. After 72 hours of incubation, the maximum growth speed is determined, the culture broth is harvested, and LBA production is analyzed on UPLC.

### Example 9. Reduced Production of Lactobionic Acid (LBA) in Modified E. coli Hosts Wherein the LBA Synthesis is Reduced

Mutant E. coli strains for the production of 2′FL, LNT, LNnT, 3′SL or 6′SL are engineered as described in Example 1. These strains are further modified with a genomic knock-out of the native aldose sugar dehydrogenase gene (GeneID: 945467), encoding the ylil enzyme (UniProt ID P75804). In a next step, these mutant strains are further modified with a genomic knock-out for the native quinoprotein glucose dehydrogenase gene (GeneID: 944830), encoding the gcd enzyme (UniProt ID P15877, sequence version 03 (2 Dec. 2020)). All novel strains together with the corresponding reference strains, having the same genetic make-up but having the native gcd gene and ylil gene still present, are evaluated in a growth experiment according to the culture conditions provided in Example 1, in which the strains are cultivated in minimal medium supplemented with 30 g/L sucrose, 20 g/L lactose and 3.30E-4 g/L pyrroloquinoline quinone (PQQ). The strains are grown in three biological replicates in a 96-well plate. After 72 hours of incubation, the culture broth is harvested, and reduced lactobionic acid (LBA) production is analyzed on UPLC in the strains having (1) a ylil knock-out and a native gcd gene or (2) a knock-out of both the ylil and gcd genes compared to the reference strains having the native ylil gene and gcd gene still present.

1.-28. (canceled)

29. A cell capable of synthesizing and/or that synthesizes lactobionic acid (4-O-β-galactopyranosyl-D-gluconic acid, LBA), a modified form of LBA, a glycosylated form of LBA, fucosylated LBA and/or sialylated LBA in the presence of lactose in the cell's cultivation or incubation medium,
wherein the cell's synthesis of the LBA, modified form of LBA, glycosylated form of LBA, fucosylated LBA and/or sialylated LBA is obtained by expressing at least one gene selected from the group consisting of a gene encoding carbohydrate oxidase, a gene encoding dehydrogenase, a gene encoding lactonase, a gene encoding oxygen-dependent FAD-linked oxidoreductase, and a gene encoding pyrroloquinoline quinone (PQQ) oxidoreductase,
wherein the cell is genetically engineered to produce a saccharide,
wherein the cell has a pathway for producing the saccharide,
wherein the synthesis of the LBA, modified form of LBA, glycosylated form of LBA, fucosylated LBA and/or sialylated LBA in the cell is rendered less functional or is knocked out by rendering less functional or knock out of the at least one gene.

30. The cell of claim 29, wherein:
the pathway for producing the saccharide is selected from the group consisting of fucosylation pathway, sialylation pathway, galactosylation pathway, N-acetylglucosaminylation pathway, N-acetylgalactosaminylation pathway, mannosylation pathway, and N-acetylmannosaminylation pathway, and/or
the pathway for producing the saccharide is selected from the group consisting of fucosylation pathway, sialylation pathway, galactosylation pathway, N-acetylglucosaminylation pathway, N-acetylgalactosaminylation pathway, mannosylation pathway, and N-acetylmannosaminylation pathway and wherein the cell is genetically engineered to comprise at least one of the pathways and/or the cell comprises at least one of the pathways wherein at least one of the pathways has been genetically engineered.

31. The cell of claim 29, wherein the cell:
possesses, expresses and/or overexpresses at least one glycosyltransferase selected from the group consisting of fucosyltransferases, sialyltransferases, galactosyltransferases, glucosyltransferases, mannosyltransferases, N-acetylglucosaminyltransferases, N-acetylgalactosaminyltransferases, N-acetylmannosaminyltransferases, xylosyltransferases, glucuronyltransferases, galacturonyltransferases, glucosaminyltransferases, N-glycolylneuraminyltransferases, rhamnosyltransferases, N-acetylrhamnosyltransferases, UDP-4-amino-4,6-dideoxy-N-acetyl-beta-L-altrosamine transaminases, UDP-N-acetylglucosamine enolpyruvyl transferases and fucosaminyltransferases,
is capable of producing and/or produces at least one nucleotide-activated sugar,
is genetically engineered for producing at least one nucleotide-activated sugar(s),
comprises a pathway for the synthesis of a nucleotide-activated sugar selected from the group consisting of UDP-N-acetylglucosamine (UDP-GlcNAc), UDP-N-acetylgalactosamine (UDP-GalNAc), UDP-N-acetylmannosamine (UDP-ManNAc), UDP-glucose (UDP-Glc), UDP-galactose (UDP-Gal), GDP-mannose (GDP-Man), GDP-fucose, (GDP-Fuc), UDP-glucuronate, UDP-galacturonate, UDP-2-acetamido-2,6-dideoxy-L-arabino-4-hexulose, UDP-2-acetamido-2,6-dideoxy-L-lyxo-4-hexulose, UDP-N-acetyl-L-rhamnosamine (UDP-L-RhaNAc or UDP-2-acetamido-2,6-dideoxy-L-mannose), dTDP-N-acetylfucosamine, UDP-N-acetylfucosamine (UDP-L-FucNAc or UDP-2-acetamido-2,6-dideoxy-L-galactose), UDP-N-acetyl-L-pneumosamine (UDP-L-PneNAC or UDP-2-acetamido-2,6-dideoxy-L-talose), UDP-N-acetylmuramic acid, UDP-N-acetyl-L-quinovosamine (UDP-L-QuiNAc or UDP-2-acetamido-2,6-dideoxy-L-glucose), CMP-sialic acid (CMP-Neu5Ac), CMP-Neu4Ac, CMP-Neu5Ac9N3, CMP-Neu4,5Ac2, CMP-Neu5,7Ac2, CMP-Neu5,9Ac2, CMP-Neu5,7(8,9)Ac2, CMP-N-glycolylneuraminic acid (CMP-Neu5Gc), GDP-rhamnose and UDP-xylose,
possesses, expresses and/or overexpresses at least one gene selected from the group consisting of mannose-6-phosphate isomerase, phosphomannomutase, mannose-1-phosphate guanylyltransferase, GDP-mannose 4,6-dehydratase, GDP-L-fucose synthase, fucose permease, fucose kinase, fucose-1-phosphate guanylyltransferase, L-glutamine D-fructose-6-phosphate aminotransferase, phosphoglucosamine mutase, N-acetylglucosamine-6-P deacetylase, N-acylglucosamine 2-epimerase, UDP-N-acetylglucosamine 2-epimerase, N-acetylmannosamine-6-phosphate 2-epimerase, UDP-GlcNAc 2-epimerase/kinase, glucosamine 6-phosphate N-acetyltransferase, N-acetylglucosamine-6-phosphate phosphatase, phosphoacetylglucosamine mutase, N-acetylglucosamine 1-phosphate uridylyltransferase, glucosamine-1-phosphate acetyltransferase, Neu5Ac synthase, N-acetylneuraminate lyase, N-acylneuraminate-9-phosphate synthase, N-acylneuraminate-9-phosphatase, sialic acid transporter, CMP kinase, CMP-sialic acid synthase, galactose-1-epimerase, galactokinase, glucokinase, galactose-1-phosphate uridylyltransferase, UDP-glucose 4-epimerase, glucose-1-phosphate uridylyltransferase, phosphoglucomutase, UDP-N-acetylglucosamine 4-epimerase, N-acetylgalactosamine kinase and UDP-N-acetylgalactosamine pyrophosphorylase, and/or
comprises a catabolic pathway for selected mono-, di- or oligosaccharides which is at least partially inactivated, the mono-, di-, or oligosaccharides being involved in and/or required for the synthesis of the saccharide.

32. The cell of claim 29, wherein the:
dehydrogenase is selected from the group consisting of lactose dehydrogenase, quinoprotein glucose dehydrogenase, aldose sugar dehydrogenase, glucose/fructose dehydrogenase, glucose/sorbosone dehydrogenase, cellobiose dehydrogenase, pyrroloquinoline quinone (PQQ) dehydrogenase, and malate dehydrogenase, and/or
carbohydrate oxidase is a lactose oxidase.

33. The cell of claim 29, wherein the at least one gene encodes an enzyme, wherein the enzyme:
is selected from the group consisting of EC:1.1.3.-, EC:1.1.3.5, EC:1.1.3.4, EC:1.1.5.-, EC:1.1.5.2, and EC:1.1.99.18,
comprises a polypeptide sequence comprising an IPR domain selected from the group consisting of IPR000172, IPR001479, IPR002372, IPR006094, IPR007867, IPR011041, IPR011042, IPR011047, IPR012132, IPR012938, IPR012951, IPR013154, IPR013428, IPR015402, IPR015920, IPR016166, IPR016169, IPR017511, IPR017512, IPR018391, IPR027424, IPR029056, IPR031640, IPR036188, and IPR036318 as defined by InterPro 90.0 as released on 4 Aug. 2022,
comprises a polypeptide sequence comprising a Panther domain selected from the group consisting of PTHR11552, PTHR13460, PTHR32303:SF4, PTHR42973 and PTHR47190 as defined by InterPro 90.0 as released on 4 Aug. 2022,
comprises a polypeptide sequence comprising a PFAM domain selected from the group consisting of PF00732, PF01011, PF05199, PF07995, PF08031, PF08240, PF13360, PF13570, PF01565, PF16010 and PF16912 as defined by InterPro 90.0 as released on 4 Aug. 2022,
comprises a polypeptide sequence comprising the conserved protein domain cd10280 as defined by InterPro 90.0 as released on 4 Aug. 2022,
is part of a NOG family selected from the group consisting of COG0277, COG2133, COG2303 and COG4993 as defined by eggNOG5.0 as released in 2019 and/or
uses a cofactor selected from the group consisting of pyrroloquinoline quinone (PQQ), FAD, a divalent metal cation, Ca2+, Cu2+, Mg2+, Zn2+, heme, heme C and pyridoxal 5′-phosphate.

34. The cell of claim 29, wherein at least one gene involved in the synthesis and/or import of:
a co-factor that is involved in the synthesis of LBA, a modified form of LBA, a glycosylated form of LBA, fucosylated LBA and/or sialylated LBA is rendered less functional or knocked out, and/or
a co-factor that is selected from the group consisting of pyrroloquinoline quinone (PQQ), FAD, a divalent metal cation, Ca2+, Cu2+, Mg2+, Zn2+, heme, heme C and pyridoxal 5′-phosphate and that is involved in the synthesis of LBA, a modified form of LBA, a glycosylated form of LBA, fucosylated LBA and/or sialylated LBA is rendered less functional or knocked out.

35. The cell of claim 29, wherein the at least one gene is rendered less functional by insertion, deletion and/or modification of one or more nucleotide(s) in one or more polynucleotide sequence(s) selected from the group consisting of promoter sequence, ribosome binding site, untranslated region, coding sequence and transcription terminator sequence of the at least one gene.

36. The cell of claim 29, wherein the cell comprises a quinoprotein glucose dehydrogenase gene that is rendered less functional by insertion, deletion and/or modification of one or more nucleotide(s) in one or more polynucleotide sequence(s) selected from the group consisting of promoter sequence, ribosome binding site, untranslated region, coding sequence and transcription terminator sequence of the quinoprotein glucose dehydrogenase gene.

37. The cell of claim 36, wherein the cell further comprises an aldose sugar dehydrogenase gene that is rendered less functional by insertion, deletion and/or modification of one or more nucleotide(s) in one or more polynucleotide sequence(s) selected from the group consisting of promoter sequence, ribosome binding site, untranslated region, coding sequence and transcription terminator sequence of the aldose sugar dehydrogenase gene.

38. The cell of claim 29, wherein the cell comprises an aldose sugar dehydrogenase gene that is rendered less functional by insertion, deletion and/or modification of one or more nucleotide(s) in one or more polynucleotide sequence(s) selected from the group consisting of promoter sequence, ribosome binding site, untranslated region, coding sequence and transcription terminator sequence of the aldose sugar dehydrogenase gene.

39. The cell of claim 38, wherein the cell further comprises a quinoprotein glucose dehydrogenase gene that is rendered less functional by insertion, deletion and/or modification of one or more nucleotide(s) in one or more polynucleotide sequence(s) selected from the group consisting of promoter sequence, ribosome binding site, untranslated region, coding sequence, and transcription terminator sequence of the quinoprotein glucose dehydrogenase gene.

40. The cell of claim 29, wherein the saccharide is selected from the group consisting of monosaccharide, phosphorylated monosaccharide, activated monosaccharide, disaccharide, oligosaccharide, neutral (non-charged) oligosaccharide, negatively charged oligosaccharide, sialylated oligosaccharide, milk oligosaccharide, mammalian milk oligosaccharide (MMO), human milk oligosaccharide (HMO), sialylated milk oligosaccharide, neutral (non-charged) milk oligosaccharide, fucosylated milk oligosaccharide, non-fucosylated neutral (non-charged) milk oligosaccharide, sialylated MMO, neutral (non-charged) MMO, fucosylated MMO, non-fucosylated neutral (non-charged) MMO, sialylated HMO, neutral (non-charged) HMO, fucosylated HMO, non-fucosylated neutral (non-charged) HMO, O-antigen, enterobacterial common antigen (ECA), the oligosaccharide repeats present in capsular polysaccharides, peptidoglycan, an amino-sugar, Lewis-type antigen oligosaccharide, an antigen of the human ABO blood group system, an animal oligosaccharide, animal oligosaccharide selected from the group consisting of N-glycans and O-glycans, a plant oligosaccharide, plant oligosaccharide selected from the group consisting of N-glycans and O-glycans, fucosylated oligosaccharide, fucosylated oligosaccharide selected from the group comprising 2′-fucosyllactose (2′FL), 3-fucosyllactose (3FL), 4-fucosyllactose (4FL), 6-fucosyllactose (6FL), 2′,3-difucosyllactose (diFL), lacto-N-fucopentaose I, lacto-N-neofucopentaose I, lacto-N-fucopentaose II, lacto-N-fucopentaose III, lacto-N-fucopentaose V, lacto-N-fucopentaose VI, lacto-N-neofucopentaose V, lacto-N-difucohexaose I, lacto-N-difucohexaose II, difucosyl-lacto-N-hexaose and difucosyl-lacto-N-neohexaose, sialylated oligosaccharide selected from the group comprising 3′sialyllactose (3′SL), 6′sialyllactose (6′SL), sialyllacto-N-tetraose a (LSTa), sialyllacto-N-tetraose b (LSTb), sialyllacto-N-tetraose c (LSTc), sialyllacto-N-tetraose d (LSTd), disialyllacto-N-tetraose, disialyllacto-N-neotetraose, monosialyllacto-N-hexaose, disialyllacto-N-hexaose I, disialyllacto-N-hexaose II, monosialyllacto-N-neohexaose I, monosialyllacto-N-neohexaose II, disialyllacto-N-neohexaose, 3′-sialyl-3-fucosyllactose, fucodisialyllacto-N-hexaose, disialomonofucosyllacto-N-neohexaose, sialyllacto-N-fucohexaose II, disialyllacto-N-fucopentaose II and monofucosyldisialyllacto-N-tetraose, N-acetylglucosamine containing neutral (non-charged) saccharide, N-acetylglucosamine containing neutral (non-charged) saccharide selected from the group comprising lacto-N-biose (LNB), N-acetyllactosamine (LacNAc), lacto-N-triose II (LN3), lacto-N-tetraose (LNT), lacto-N-neotetraose (LNnT), 6′-galactosyllactose, 3′-galactosyllactose, lacto-N-hexaose, lacto-N-neohexaose, para-lacto-N-hexaose, para-lacto-N-neohexaose, N-acetylglucosamine containing saccharide, N-acetyllactosamine containing saccharide, lacto-N-biose containing saccharide, non-fucosylated neutral (non-charged) saccharide, chitosan, chitosan comprising oligosaccharide, heparosan, chondroitin sulphate, glycosaminoglycan oligosaccharide, heparin, heparan sulphate, dermatan sulphate, hyaluronan, hyaluronic acid, and keratan sulphate.

41. The cell of claim 29, wherein the cell:
is capable of producing and/or produces the saccharide, LBA, modified form of LBA, glycosylated form of LBA, fucosylated LBA, and/or sialylated LBA from one or more precursor(s),
is capable of producing and/or produces the saccharide, LBA, modified form of LBA, glycosylated form of LBA, fucosylated LBA, and/or sialylated LBA from lactose,
is capable of producing and/or produces at least one precursor that is used to produce the saccharide, LBA, modified form of LBA, glycosylated form of LBA, fucosylated LBA and/or sialylated LBA,
is capable of producing and/or produces all precursors that are used to produce the saccharide, LBA, modified form of LBA, glycosylated form of LBA, fucosylated LBA and/or sialylated LBA,
is genetically engineered for the production of at least one precursor that is used to produce the saccharide, LBA, modified form of LBA, glycosylated form of LBA, fucosylated LBA and/or sialylated LBA, and/or
is genetically engineered for the production of all precursors that are used to produce the saccharide, LBA, modified form of LBA, glycosylated form of LBA, fucosylated LBA and/or sialylated LBA.

42. The cell of claim 41, wherein at least one of the one or more precursors is internalized in the cell via one or more membrane protein(s).

43. The cell of claim 29, wherein the cell:
is selected from prokaryotic cells and eukaryotic cells,
is selected from the group consisting of yeast cells, bacterial cells, archaebacterial cells, algae cells, plant cells and fungal cells,
is an E. coli or yeast with a lactose permease positive phenotype, and/or
is an E. coli or yeast with a lactose permease positive phenotype wherein the lactose permease is coded by the gene LacY or LAC12, respectively.

44. A method of producing a saccharide, the method comprising:
i. cultivating and/or incubating the cell of claim 29 in cultivation and/or incubation medium under conditions permissive to produce the saccharide and any one or more of LBA, a modified form of LBA, a glycosylated form of LBA, fucosylated LBA, and sialylated LBA, and
ii. (a) separating the saccharide from the cultivation and/or incubation, and/or
(b) separating the saccharide from the LBA, modified form of LBA, glycosylated form of LBA, fucosylated LBA and/or sialylated LBA.

45. The method according to claim 44, wherein the cultivation or incubation medium comprises at least one precursor that is used for producing the saccharide, LBA, modified form of LBA, glycosylated form of LBA, fucosylated LBA and/or sialylated LBA.

46. The method according to claim 44, wherein the cell produces 30 g/L or more of the saccharide in whole broth and/or supernatant and/or wherein the saccharide in the whole broth and/or supernatant has a purity of at least 80% measured on the total amount of saccharide and its precursor(s) produced by the cell in the whole broth and/or supernatant, respectively.

47. The method according to claim 44, wherein the less functional or knocked out synthesis of LBA, modified form of LBA, glycosylated form of LBA, fucosylated LBA and/or sialylated LBA confers unaffected and/or enhanced i) saccharide formation, ii) productivity, iii) biomass production, iv) cell growth and/or v) yield of the produced saccharide, relative to a corresponding non-modified cell.

48. The method according to claim 44, wherein the cell produces a mixture comprising the saccharide and any one or more of the LBA, modified form of LBA, glycosylated form of LBA, fucosylated LBA and/or sialylated LBA wherein the mixture comprises≤10 weight % of the LBA, modified form of LBA, glycosylated form of LBA, fucosylated LBA and/or sialylated LBA, ≤9 weight % of the LBA, modified form of LBA, glycosylated form of LBA, fucosylated LBA and/or sialylated LBA, ≤8 weight % of the LBA, modified form of LBA, glycosylated form of LBA, fucosylated LBA and/or sialylated LBA, ≤7 weight % of the LBA, modified form of LBA, glycosylated form of LBA, fucosylated LBA and/or sialylated LBA, ≤6 weight % of the LBA, modified form of LBA, glycosylated form of LBA, fucosylated LBA and/or sialylated LBA, ≤5 weight % of the LBA, modified form of LBA, glycosylated form of LBA, fucosylated LBA and/or sialylated LBA, ≤4 weight % of the LBA, modified form of LBA, glycosylated form of LBA, fucosylated LBA and/or sialylated LBA, ≤3 weight % of the LBA, modified form of LBA, glycosylated form of LBA, fucosylated LBA and/or sialylated LBA, ≤2 weight % of the LBA, modified form of LBA, glycosylated form of LBA, fucosylated LBA and/or sialylated LBA, ≤1 weight % of the LBA, modified form of LBA, glycosylated form of LBA, fucosylated LBA and/or sialylated LBA, ≤0.5 weight % of the LBA, modified form of LBA, glycosylated form of LBA, fucosylated LBA and/or sialylated LBA and/or ≤0.1 weight % of the LBA, modified form of LBA, glycosylated form of LBA, fucosylated LBA and/or sialylated LBA.

49. The method according to claim 44, wherein the cell produces no LBA.

50. The method according to claim 44, wherein the saccharide is recovered from the cultivation or incubation medium and/or the cell, and/or wherein the saccharide is purified.

51. The method according to claim 44, wherein the saccharide is purified from (i) the LBA and/or from (ii) the modified form of LBA, glycosylated form of LBA, fucosylated LBA and/or sialylated LBA.

52. A method of using the cell of claim 29 to produce a saccharide, wherein the saccharide is selected from the group consisting of monosaccharide, phosphorylated monosaccharide, activated monosaccharide, disaccharide, oligosaccharide, neutral (non-charged) oligosaccharide, negatively charged oligosaccharide, sialylated oligosaccharide, milk oligosaccharide, mammalian milk oligosaccharide (MMO), human milk oligosaccharide (HMO), sialylated milk oligosaccharide, neutral (non-charged) milk oligosaccharide, fucosylated milk oligosaccharide, non-fucosylated neutral (non-charged) milk oligosaccharide, sialylated MMO, neutral (non-charged) MMO, fucosylated MMO, non-fucosylated neutral (non-charged) MMO, sialylated HMO, neutral (non-charged) HMO, fucosylated HMO, non-fucosylated neutral (non-charged) HMO, O-antigen, enterobacterial common antigen (ECA), the oligosaccharide repeats present in capsular polysaccharides, peptidoglycan, an amino-sugar, Lewis-type antigen oligosaccharide, an antigen of the human ABO blood group system, an animal oligosaccharide, an animal oligosaccharide selected from the group consisting of N-glycans and O-glycans, a plant oligosaccharide, a plant oligosaccharide selected from the group consisting of N-glycans and O-glycans, fucosylated oligosaccharide, fucosylated oligosaccharide selected from the group comprising 2′-fucosyllactose (2′FL), 3-fucosyllactose (3FL), 4-fucosyllactose (4FL), 6-fucosyllactose (6FL), 2′,3-difucosyllactose (diFL), lacto-N-fucopentaose I, lacto-N-neofucopentaose I, lacto-N-fucopentaose II, lacto-N-fucopentaose III, lacto-N-fucopentaose V, lacto-N-fucopentaose VI, lacto-N-neofucopentaose V, lacto-N-difucohexaose I, lacto-N-difucohexaose II, difucosyl-lacto-N-hexaose and difucosyl-lacto-N-neohexaose, sialylated oligosaccharide selected from the group comprising 3′sialyllactose (3′SL), 6′sialyllactose (6′SL), sialyllacto-N-tetraose a (LSTa), sialyllacto-N-tetraose b (LSTb), sialyllacto-N-tetraose c (LSTc), sialyllacto-N-tetraose d (LSTd), disialyllacto-N-tetraose, disialyllacto-N-neotetraose, monosialyllacto-N-hexaose, disialyllacto-N-hexaose I, disialyllacto-N-hexaose II, monosialyllacto-N-neohexaose I, monosialyllacto-N-neohexaose II, disialyllacto-N-neohexaose, 3′-sialyl-3-fucosyllactose, fucodisialyllacto-N-hexaose, disialomonofucosyllacto-N-neohexaose, sialyllacto-N-fucohexaose II, disialyllacto-N-fucopentaose II and monofucosyldisialyllacto-N-tetraose, N-acetylglucosamine containing neutral (non-charged) saccharide, N-acetylglucosamine containing neutral (non-charged) saccharide selected from the group comprising lacto-N-biose (LNB), N-acetyllactosamine (LacNAc), lacto-N-triose II (LN3), lacto-N-tetraose (LNT), lacto-N-neotetraose (LNnT), 6′-galactosyllactose, 3′-galactosyllactose, lacto-N-hexaose, lacto-N-neohexaose, para-lacto-N-hexaose, para-lacto-N-neohexaose, N-acetylglucosamine containing saccharide, N-acetyllactosamine containing saccharide, lacto-N-biose containing saccharide, non-fucosylated neutral (non-charged) saccharide, chitosan, chitosan comprising oligosaccharide, heparosan, chondroitin sulphate, glycosaminoglycan oligosaccharide, heparin, heparan sulphate, dermatan sulphate, hyaluronan, hyaluronic acid, and keratan sulphate.

53. A mixture comprising:
(i) a saccharide and
(ii) LBA, a modified form of LBA, a glycosylated form of LBA, fucosylated LBA and/or sialylated LBA
wherein the saccharide, LBA, modified form of LBA, glycosylated form of LBA, fucosylated LBA and/or sialylated LBA is obtained by the method according to claim 44.

54. The mixture of claim 53, wherein the mixture comprises≤10 weight % LBA.

55. A mixture comprising:
a saccharide and
(i) ≤10 weight % LBA, and/or
(ii) ≤10 weight % of a modified form of LBA, a glycosylated form of LBA, fucosylated LBA and/or sialylated LBA,
wherein the saccharide, LBA, modified form of LBA, glycosylated form of LBA, fucosylated LBA and/or sialylated LBA is obtained by the method according to claim 44.
